(19) United States
(12) Patent Application Publication
(10) Pub. No.: US 2013/0040291 A1
(43) Pub. Date: Feb. 14, 2013

(54) Detection and Quantification of Cellular Stress Response Resolution Pathway Expression and Functionality and the Effects of Agents or Conditions Thereupon

(71) Applicants: Dale M. WALKER, Jericho, VT (US); Vernon E. WALKER, Jericho, VT (US)

(72) Inventors: Dale M. WALKER, Jericho, VT (US); Vernon E. WALKER, Jericho, VT (US)

(21) Appl. No.: 13/651,371

(22) Filed: Oct. 12, 2012

Related U.S. Application Data
(63) Continuation-in-part of application No. PCT/US2011/032191, filed on Apr. 12, 2011.

(51) Int. Cl. C12Q 1/18; C12Q 1/68; C12Q 1/02

(52) U.S. Cl. 435/6.1; 435/32; 435/29

(57) ABSTRACT

The assessment methods disclosed herein involve exposing cells to two stresses. The first stress (analogous to existing direct screening methods) involves exposing metazoan cells to an external stimulus of interest, such as exposing human cells to ionizing radiation, a chemical, or a set of physical conditions. The cells exposed to the first stress are thereafter exposed to a second stress which, importantly, is calibrated such that non-stressed cells of the same type will exhibit a predictable response to the second stress. By comparing the response to the second stress of i) cells that were exposed to the first stress and ii) cells that were not exposed to the first stress, an observer can assess whether exposure to the first stress caused or induced any change to the cells that affected the cells' ability to respond to the second stress.

### CROSS-REFERENCE TO RELATED APPLICATIONS

This application is a continuation-in-part of international patent application PCT/US2011/032191, filed 12 Apr. 2011 which is entitled to the benefit of the filing date of U.S. provisional patent application No. 61/323,307, filed 12 Apr. 2010; each of the foregoing applications is incorporated herein by reference in its entirety.

### BACKGROUND OF THE DISCLOSURE

The disclosure relates generally to methods of assessing the effects of external stimuli on the ability of metazoan cells to effectively respond to stress.

It is well known to assess the direct effects of external stimuli on metazoan cells. Such screening assays are routinely used to assess the effects of stimuli such as various forms of irradiation, temperature insult, contact with exogenous chemicals, and exposure to etiological agents for various diseases and conditions. Routine screening assays involve exposing metazoan cells to one or more of the stimuli, and thereafter observing gross phenotypic effects (e.g., cell death or cell cycle arrest), molecular phenotypic effects (e.g., changes in cell-surface protein display, changes in enzyme production, or changes in gene expression), or genotypic effects (e.g., mutations in genomic or mitochondrial DNA) of the exposure on the exposed cells. Although observations from such ‘direct’ screening assays can yield much useful information about the acute toxicity of the stimuli, direct screening assays are usually not informative of subtler, less directly observable changes made to or induced in exposed cells.

Changes made to or induced in cells by exposure to an external stimulus can have serious consequences, especially if those changes are not well understood. By way of example, rofecoxib (previously marketed under the trade name VIOXX by Merck & Co., Whitehouse Station, N.J.) is a pharmaceutically active agent having numerous beneficial effects in humans. Direct screening assays assessing toxicity of rofecoxib yielded data which led to approval of rofecoxib for sale by the U.S. Food and Drug Administration. As experience with the drug accumulated, it was observed that use of the drug appeared to lead to damage in cardiovascular tissues, the severity and incidence of which was sufficient to prompt a voluntary recall of the drug by its manufacturer. Had the ability of the drug to render cardiovascular tissue susceptible to injury been more fully understood during its development, cardiovascular damage in numerous patients might have been avoided.

Pre-marketing studies of drugs and other chemicals are not capable of revealing at least some dangers inherent in potentially cytodamaging agents. Similarly, the effects of non-chemical stimuli (e.g., radiation exposure, exposure to electromagnetic fields, and temperature, acceleration, shear force, and pressure effects) may not be fully appreciable through observation of the direct effects of such stimuli upon cells. A significant need exists for methods of assessing indirect effects of potentially harmful (and potentially harm-preventing) stimuli upon metazoan (especially human) cells. The present disclosure describes such methods.

### BRIEF SUMMARY OF THE DISCLOSURE

The assessment methods disclosed herein involve exposing cells to two stresses. The first stress (analogous to existing direct screening methods) involves exposing metazoan cells to an external stimulus of interest, such as exposing human cells to ionizing radiation, a chemical, or a set of physical conditions. The cells exposed to the first stress are thereafter exposed to a second stress which, importantly, is calibrated such that non-stressed cells of the same type will exhibit a predictable response to the second stress. By comparing the response to the second stress of i) cells that were exposed to the first stress and ii) cells that were not exposed to the first stress, an observer can assess whether exposure to the first stress caused or induced any change to the cells that affected the cells' ability to respond to the second stress. Thus, rather than (or, more correctly, in addition to) merely enabling an observer to observe direct effects of the first stress on the cells, the observer is also enabled to observe how, if at all, the first stress modulates (i.e., enhanced or detrimented) one or more stress responses of the cells, such that the cells' response to a second incident of stress is affected.

By way of example, a prior direct screening assay of a pharmaceutically active agent may indicate that the agent exerts no direct cytotoxic or cytodamaging effects on metazoan cells of a certain type. If the agent inhibits the ability of exposed cells to respond to further stress, prior direct screening assays will often not reveal that fact. Performance of the stress response assays described herein, by contrast, will reveal the effects of the agent on the ability of exposed cells to deal with further stress (in addition to direct effects of the agent upon the cells). The assays described herein therefore represent an important advance over the prior art of screening stimuli for harmful or beneficial effects upon metazoan cells.

Prior to this disclosure, it was known that metazoan cells have numerous systems (described in greater detail in this disclosure and in the prior art) for continuing, discontinuing, or altering their viability and function in responses to changes in stimuli experienced by such cells. It was furthermore known that these various stress response resolution (SRR) pathways can lead to certain molecular and physiological changes in cells, with the particular changes being determined in part by activation of one more SRR pathways. Because such changes can alter the susceptibility of the cells to external stimuli, the ability to identify or predict which SRR pathway(s) will be induced or active in a metazoan cell enables one to predict the effects of the external stimuli upon the cell. It is, in part, to this end that the methods disclosed herein are directed.

This disclosure is in the fields of basic medical science including cell and molecular biology, toxicology including risk and/or safety assessment, and pharmacology, including drug discovery, development, efficacy and safety testing. More specifically, this disclosure pertains to methods for quantification of stress response resolution pathway expression and functionality, including but not limited to quantification of DNA mutations, epigenetic alterations, and loss of epigenetic-based cellular memory maintenance (ECM) and restoration functions in human and other mammalian cells, including but not limited to epithelial cells of any organ or tissue, neurons of any type, glial cells of any type, cardiomyocytes, cells of the immune system including dendritic cells, stem cells including embryonic stem cells, stromal cells of any type, and endothelial cells.

Specifically, this disclosure pertains to an in vitro cell culture assay, termed the stress response resolution or SR assay, which can be used to evaluate the outcomes of cellular stress resolution pathways. The SR assay described herein uses a unique step, referred to as the ‘calibrated stress conditions step’ to induce a moderate level stress response in cells of interest for the purpose of quantifying cellular stress resolution pathway outcomes. The design of the SR assay conforms to Poisson distribution principles and application of the assay results in raw data, which is binomial in nature, and assay-derived materials that can be subjected to additional evaluations. The SR assay can be applied to a wide variety of human and animal cells types, including but not limited to epithelial cells, and can be used to test the effects of exogenous exposures upon cellular stress resolution outcomes in these cells types.

This disclosure also pertains to the use of these methods for (i) investigation of mechanisms of disease pathogenesis, (ii) identification of susceptibility/resistance factors that predispose individuals or groups to disease occurrence, and (iii) identification of chemicals, drugs, compounds, substances, physical agents or other conditions (test agent(s) or conditions) that alter, beneficially or deleteriously, the expression, functionality and/or outcome of one or more cellular stress response resolution pathways and/or ECM maintenance and/or restoration pathways or systems.

Test agents or conditions to be evaluated using the SR assay can be applied to the cells in vivo in animals or in vitro in tissue culture. Cellular stress responses that can be evaluated in this assay include but are not limited to those stress responses that are induced by agents or conditions that are resolved by cells through expression of predominantly error-free (damage avoidance) or error-prone (damage tolerance) pro-survival pathways. These stress responses include but are not limited to agents or conditions that induce DNA, chromatin or epigenetic damage, alterations, or stress, cytotoxic stress, hypoxic stress, oxidant stress, nutrient deprivation stress, environmental stress (example heat, cold, pressure, shearing forces, osmotic changes), and any other stress that is resolved using the above pathways. The present disclosure pertains to methods for identifying and quantifying the effect(s) of an exogenous exposure upon the expression and functionality of three major groups of stress response resolution pathways, including (i) damage avoidance pathways that result in cell protective, predominantly error-free, and/or non-mutagenic stress response resolution outcomes, (ii) damage tolerance pathways that result in error-prone-reversible stress response resolution outcomes, and (iii) damage tolerance pathways that result in error-prone-irreversible stress response resolution outcomes.

The disclosure also pertains to evaluation of damage tolerance stress response resolution through quantification of alterations in both the epigenome and DNA (reversible and irreversible alterations are assessed).

The disclosure also pertains to methods for identifying test agents or conditions that induce genetic and/or epigenetic alterations resulting in expression of a tumorigenic phenotype and for identifying agents that prevent, mitigate or protect against induction of error-prone (damage tolerance) stress response resolution pathways and/or expression of the tumorigenic phenotype.

The subject matter of this disclosure relates to a method of assessing the potential of an external stimulus to modulate a stress response resolution pathway (SRRP) in a metazoan cell of a selected type. The method includes at least three aspects: A) subjecting a metazoan cell of the selected type to a first incident of the stimulus; thereafter B) subjecting the cell to a calibrated stress condition (CSC) wherein the CSC is selected such that a substantial fraction of control cells of the same type that have not been subjected to any substantial stress exhibit a reproducible response to the CSC; and thereafter C) assessing the difference between i) the response to the CSC of the cell subjected to the stimulus and ii) the response to the CSC of the control cells. A difference between i) and ii) indicates that the stimulus modulates stress response in cells of the selected type.

The SRRP can be selected from the group consisting of pro-survival, damage avoidance (PSDA) SRRPs; pro-survival damage tolerance (PSDT) SRRPs; and pro-death SRRPs. In the assay, the difference between i) the response to the CSC of the cell subjected to the stimulus is a characteristic of the selected SRRP in the cell and ii) the response to the CSC of the control cells is the same characteristic of the selected SRRP in the control cells can be assessed.

Examples of characteristic that can be assessed include characteristics of mitochondrial alterations; characteristics of DNA mutation processes; characteristics of epigenome alterations; characteristics of RNA-directed DNA methylation processes; characteristics of DNA demethylation processes; characteristics of Histone H3K9 methylation processes; characteristics of RNA-directed histone modification processes; characteristics of RNA-directed nucleosome repositioning processes; characteristics of RNA-directed chromatin remodeling processes; characteristics of transcriptional state modification processes; characteristics of RNA editing processes modulated by intracellular transport and modulation of RNA-containing components; characteristics of DNA editing processes modulated by intracellular transport and modulation of RNA-containing components; characteristics of processes for propagation of chromatin structure; characteristics of processes for propagation of chromatin configurations; characteristics of processes for maintenance of bivalent historic modifications; characteristics of chromatin bookmarking processes; characteristics of immune system antigenic memory processes; characteristics of processes for expression of polycomb group proteins; characteristics of processes for binding of polycomb group proteins with nucleic acids; characteristics of processes for micronRNA-mediated gene expression; characteristics of processes for expression of small non-coding nucleic acids; and characteristics of processes for expression of ribonucleoproteins. For example, if the SRRP is a pro-death SRRP and the characteristic can be cell survival or cell death.

A direct effect of the stimulus on the cell can be assessed after subjecting it to the stimulus and prior to subjecting it to the CSC.

In one aspect, a plurality of cells of the same type are subjected to the stimulus prior to subjecting the plurality of cells to the CSC. Preferably, substantially all of the cells of the plurality are present in the form of single cells. Cells that exhibit a difference between i) and ii) can be isolated from the plurality after the plurality is subjected to the CSC.

In one embodiment of the assay, the response of control cells to the CSC is substantially the same among all control cells.

In another aspect, a characteristic is assessed collectively for groups of control cells and the response of the groups to the CSC is that about half of the groups exhibit one binomial state of the characteristic. The remainder of the groups exhibit the other binomial state of the characteristic. In an analogous embodiment, a characteristic is assessed collectively for groups of control cells and the response of the groups to the CSC is that at least about a third of the groups exhibit one binomial state of a characteristic. The remainder of the groups exhibit the other binomial state of the characteristic. In yet another embodiment, the response of control cells to the CSC is that at least about a tenth of the control cells exhibit one binomial state of a characteristic. The remainder of the control cells exhibit the other binomial state of the characteristic.

The external stimulus that is studied using the assay can be contact with a composition, such as one that includes a pharmaceutically active agent. The stimulus can also be exposure to an agent suspected of having a deleterious effect on health of the metazoan, or exposure to an agent suspected of having a beneficial effect on health of the metazoan.

Examples of types of cells that can be used in the assay include glial cells, sensory neurons, motor neurons, interneurons, cardiomyocytes, dendritic cells, fibroblasts, fibrocytes, multipotent cells, endothelial cells of a single organ, endothelial cells of a single tissue, and epithelial cells. Other examples include primary rat lung epithelial cells, immortalized murine mammary epithelial cells, immortalized murine alveolar type II lung cells, and immortalized human bronchial epithelial cells.

Examples of SRRPs that can be studied using the assay include pathways that resolves a stress response (SR) selected from the group consisting of genotoxic SRs, cytotoxic SRs, hypoxic SRs, oxidant SRs, heat SRs, cold SRs, osmotic SRs, nutrient SRs, protein misfolding SRs, cell crowding SRs, physical pressure SRs, and physical shear SRs. The SRRP can also be is selected from the group consisting of a pathway that modulates epigenetic changes, a pathway that modulates DNA changes, a pathway that modulates chromatin changes, and a pathway that modulates mitochondrial changes.

### BRIEF SUMMARY OF THE SEVERAL VIEWS OF THE DRAWINGS

FIG. 1A is an illustration of a paradigm. The illustrated paradigm (a) shows a few of the essential steps hypothesized to lead from exposure to an exogenous agent (and/or induction of stress) to mutagenesis, epigenetic alterations, altered gene expression, altered cell function, and/or disease, in this paradigm, two major avenues are hypothesized to serve as initiating or inducing events; these processes include exposure to an exogenous agent or condition that damages DNA, or exposure to an agent or condition that results in alterations to the epigenome or other cellular components, which then result in DNA damage through indirect effects. The key event in this paradigm is the induction of DNA damage. Once this has occurred, genotoxic effects including but not limited to mutagenesis occur, and are modeled as being an obligatory outcome of the exposure and/or damage induction. These events then result in altered cell function and disease. One of the most heavily studied diseases resulting from these events is cancer.

FIG. 1B is an illustration of a paradigm. This paradigm (B) illustrates a few of the steps of a model that represents an alternative to that shown in FIG. 1A. As above, only a few essential steps have been shown due to space limitations. The steps shown in FIG. 1A are present in this alternative model, but additional steps also are included and the outcomes have been expanded considerably. In FIG. 1B, at least three major avenues are hypothesized to serve as initiating or inducing events that can lead to altered cell function and/or disease. These avenues consist of (1) exogenous exposure(s) that result in damage or change to one or more of multiple cellular targets (see list at bottom of figure) that then result in stress signaling, (2) exposure to a stress environment, which usually occurs in the form of an environmental change, and that damages or changes one or more cellular components, thereby resulting in stress signaling, and (3) exposure to agents or conditions that have the capacity to function as stress signaling agents or stress signaling-mimetic agents. An example of the latter is exposure to reactive oxygen species. Note that the ‘indirect’ pathway depicted in paradigm A is incorporated into several of the pathways depicted in paradigm B. The central event in this paradigm is the induction of stress signaling; this signaling can originate within a damaged or stressed cell, but it also can originate in a neighboring cell(s) that then communicate stress to surrounding cells (bystander effect(s)). In other words, the stress signals shown in FIG. 1B may have an intra- or inter-cellular origin. These signals are critical to the paradigm because they link the level and duration of an exogenous exposure or stress environment experienced by cells or cell populations to a variety of potential outcomes. At least 4 major groups of potential outcomes are presented; regulation of expression of these outcomes is postulated to be governed by thresholds. In general, the level of stress signaling increases with the severity and duration of the damage and/or stress environment, but a few exceptions have been noted (Bartek et al., 2007b). In general, the outcomes progress from #1 to #4 and reflect increasing strength and duration of stress signaling, with thresholds for each transition between groups of pathways leading to the outcomes. The threshold levels vary by factors that affect stress responses (see background). In this paradigm, mutagenesis, epigenetic alterations, altered cell function, and/or disease are not obligatory outcomes of an exogenous exposure or a stress environment; these deleterious results only are associated with higher and/or more prolonged levels of stress signaling. Paradigm B shown in FIG. 1B is novel and was developed as a result of the need to describe SR assay results. It is supported by work performed with bacteria and yeast along with a few studies of mammalian or human cells.

FIG. 2 is a time line diagram for the SR assay, showing major steps of the assay and the timing of their performance relative to each other. The time line is indicated by the black line near the center of the figure. Exposures are represented above the timeline and assessments or evaluations are represented below the timeline. There are two exposures: (1) indicates the initial exposure that can be performed if desired as part of the assay, (2) indicates the calibrated stress conditions exposure that is an essential step in the assay. Note that the initial exposure is not limited in time and can extend for anytime period desired. There are two assessment periods: (A) the midway assessments can be performed at any time up to the start of the calibrated stress conditions exposure, (B) the final assessments can be started at any time after the calibrated stress conditions exposure and the timing of their completion will depend upon the time required to collect the raw data derived from the assay. This will vary with cell type, calibrated stress inducing agent, and other factors specific to each experimental design.

FIG. 3A is a bar graph that illustrates results of a pilot study using CDNR4 cells to determine the calibrated stress response induction conditions necessary to achieve a level 50% (SRID-50%), defined as the level of stress inducing agent that reduces cell survival of wild-type, parental-type and/or vehicle-exposed cells to one clone or fewer in 50% of the wells of a 96 well plate, or to comparable levels in other tissue culture vessels, when the cells are plated at a low cell density (see determination of calibrated stress conditions methods). Note that the assay can be used with any level of stress response induction level, but the SRID-50% level was found to be informative for a wide range of stress response outcomes, including but not limited to expression of damage avoidance paths, and thus, to be useful for assessment of the expression and functionality of these outcomes. In this example, overall cell survival was reduced to 1 viable clone in approximately 50% of the wells of a 96-well tissue culture plate when the cells were plated at a low plating density and exposed to 35 micrograms of 6-TG for four days. This was defined as the SRID-50% for CDNR4 cells plated at low density and exposed to 6-TG for 4 days.

FIG. 3B is a bar graph that illustrates results of a pilot study to evaluate the effects of a pre-exposure to ENU upon SRID-50% levels for CDNR4 cells, CDNR4 cells were exposed to 20 micrograms ethylnitrosourea (ENU) for one hour. Two weeks later, the cells were tested to determine the SRID-50% level. The graph shows that the SRID-50% level of ENU-exposed CDNR4 cells was reduced to an average of 2.5 micrograms 6-TG for 4 days. This represents a 20-fold reduction in the functionality of pro-survival cellular stress response pathways.

FIG. 4A is a bar graph that illustrates pre-stress-induction CEs (as indicative of pro-survival pathway functionality under non-stress conditions) and post-stress-induction SFs (also termed CEs) for CDNR4 cells exposed to vehicle alone or to ENU. CDNR4 cells were exposed to vehicle or to 20 micrograms/ml of ENU for 1 hour. Following a 2-week phenotypic expression period (to allow for stress response adaptation and/or resolution), overall cell survival was assessed using the cloning assay (technique B) for determination of pre-stress-induction survival capacity (CEs) in epithelial cells. There was no significant difference between vehicle-exposed versus ENU-exposed cells. In other words, these results show that exposure to ENU did not have a significant effect upon clonal cell survival under the low level stress conditions of in vitro cell culture.

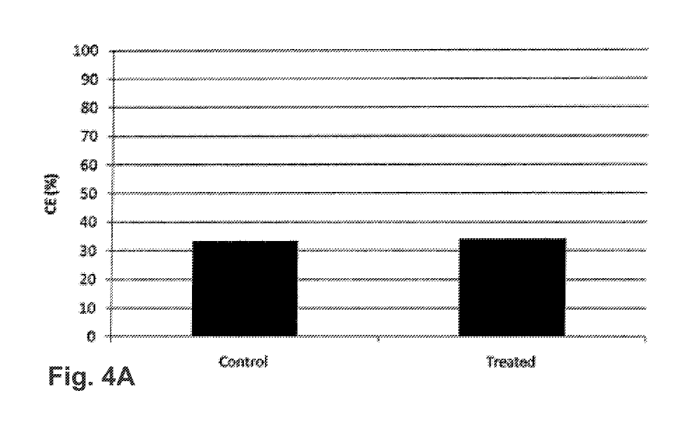

FIG. 4B is a bar graph that illustrates post-SR-induction SFs (CEs) for CDNR4 cells pre-exposed to vehicle (controls) versus ENU. These results show that the pre-exposure to ENU significantly reduced the total survival of CDNR4 cells following induction of a 6-TG-mediated stress response. These results show that the ENU exposure had a significant deleterious effect upon the functionality of stress resolution pro-survival pathways; this finding also represents a novel result for the well-studied mutagen ENU, and suggests additional modes of action for this compound that have not been described previously. Note that comparisons between pre-SR induction and post-SR-induction survival frequencies (CEs) cannot be made based using these data, because the techniques used are sufficiently different that they affect comparisons, and the formulas used to calculate the total survival percentages and frequencies differ between the techniques. If such a comparison is desired, it is necessary to pool the clones that survive post-SR induction for treated cells and for control cells, and repeat the cloning efficiency studies as performed before the SR-induction phase of the assay. These comparisons are made when surviving cell population composition is evaluated.

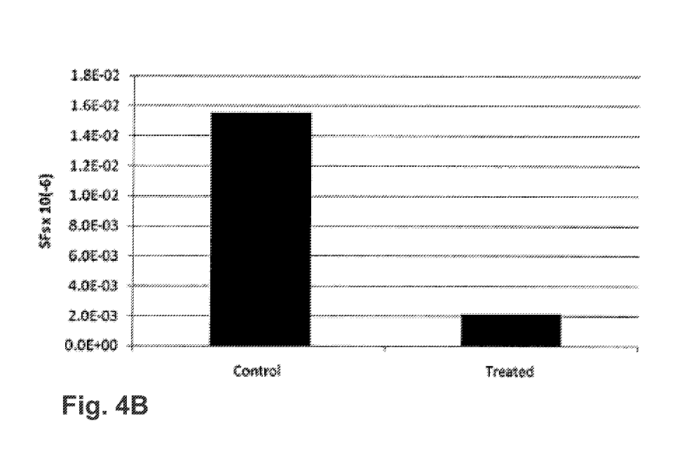

FIG. 5 is a bar graph that illustrates comprehensive results for the SR assay performed using CDNR4 cells exposed to vehicle or to 2.0 micrograms/ml of ENU for 1 hour. Following a 2-week phenotypic expression period (stress resolution and/or adaptation period), the SR assay was performed. Vehicle- (gray bar) or to ENU- (black bars) exposed cells are shown. Note that only one response type was observed for vehicle-exposed cells; these cells were classified as phenotype-parental (Phen-parental) based upon the criteria shown in Table 6. For these vehicle exposed cells, the frequency of occurrence (CEs) of Phen-HPRT⁻, Phen-alt-rev, and/or Phen-alt-irr clones were below 1×10⁻⁶ (the limit of detection for the SR assay in these experiments). In contrast, four types of surviving cell clones were observed among cells exposed to ENU. The Phen-parental CEs for vehicle exposed cells were 3,400±120×10⁻⁶. The CEs and mutant frequencies for the Phen-parental, Phen-HPRT⁻, Phen-alt-rev, and/or Phen-alt-irr clones among ENU exposed cells were 95±20×10⁻⁶, 160±40×10⁻⁶, 180±130×10⁻⁶, and 690±170×10⁻⁶, respectively. Note that the Phen-HPRT⁻ mutant frequency for ENU exposed cells of 160×10⁻⁶ is comparable to the HPRT⁻ mutant frequency obtained in peripheral blood lymphocytes exposed to ENU (Dobrovsky et al, 1999).

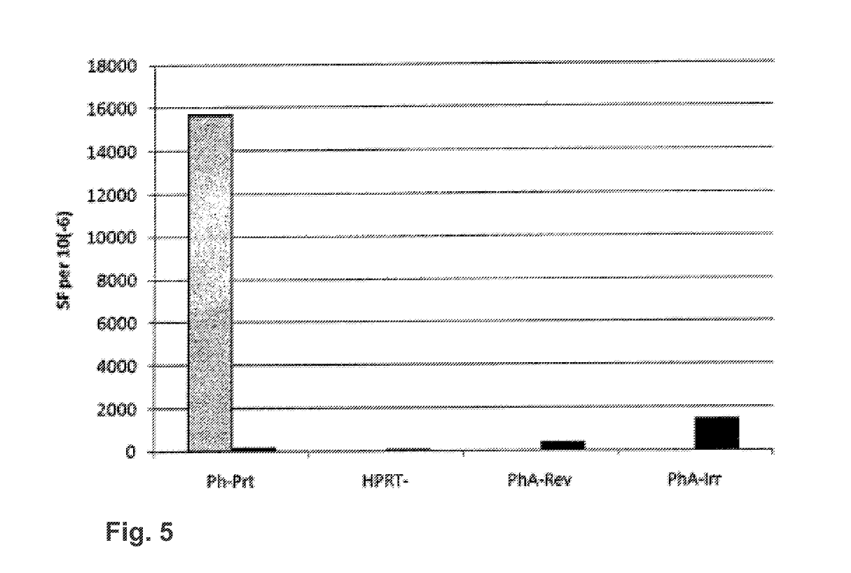

FIG. 6 is a chart that illustrates the range of comparisons that can be done using SR assay-derived data and materials, in this figure ‘Ph’ is used as an abbreviation for ‘phenotype’. Black boxes represent comparisons between identical endpoints, and boxes containing ‘R’ represent repeated comparisons shown elsewhere in the table. A total of 21 comparisons can be performed to obtain additional information about results obtained using the SR assay.

FIG. 7 is a diagram that illustrates a known assay for assessing the response of cells to a first stressor.

FIG. 8 is a diagram that illustrates a known assay for assessing the response of cells to a second stressor.

FIG. 9 is a diagram that illustrates pathways and phenotypic states discussed herein exhibited by cells that have been exposed to a first stressor.

FIG. 10 is a diagram that illustrates pathways and phenotypic states discussed herein exhibited by cells that have been exposed to both a first stressor and a second stressor.

### DETAILED DESCRIPTION

The disclosure relates to methods of assessing characteristics of stress response resolution pathway(s) in a metazoan cell.

The assessment methods disclosed herein involve exposing cells to two stresses. The first stress (analogous to existing direct screening methods) involves exposing metazoan cells to an external stimulus of interest, such as exposing human cells to ionizing radiation, a chemical, or a set of physical conditions. The cells exposed to the first stress are thereafter exposed to a second stress which, importantly, is calibrated such that non-stressed cells of the same type will exhibit a predictable response to the second stress. By comparing the response to the second stress of i) cells that were exposed to the first stress and ii) cells that were not exposed to the first stress, an observer can assess whether exposure to the first stress caused or induced any change to the cells that affected the cells' ability to respond to the second stress. Thus, rather than (or, more correctly, in addition to) merely enabling an observer to observe direct effects of the first stress on the cells, the observer is also enabled to observe how, if at all, the first stress modulates (i.e., enhanced or detrimented) one or more stress responses of the cells, such that the cells' response to a second incident of stress is affected.

Introduction to the Subject Matter of this Disclosure

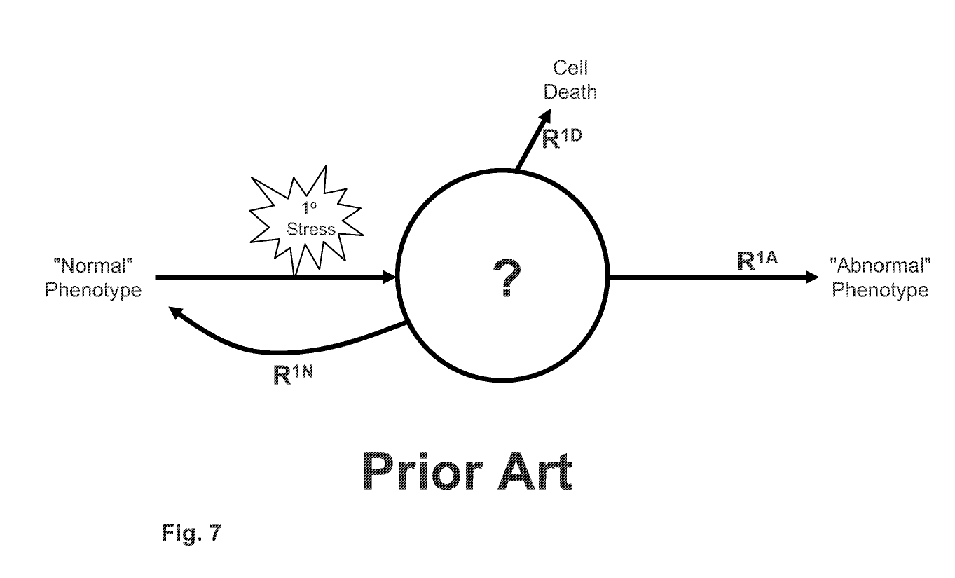

Some of the key findings underlying development of the methods and kits described herein are illustrated in FIGS. 7-10.

In FIG. 7, cells exhibiting a “normal” (i.e., baseline) phenotype are exposed to the first stressor (e.g., a chemical agent, radiation, heat, cold, hydrostatic pressure, or a biological agent). Immediately to shortly after exposure, the status of the exposed cells is unknown. After a selected time, the cells can be assessed and will exhibit one of three outcomes: normal phenotype, “abnormal” phenotype (i.e., some phenotype different than the initial normal phenotype), or dead/dying. The relative rates or proportions at which cells exhibit these outcomes can be described (e.g., factors R^{1N}, R^{1A}, and R^{1D}, respectively, in FIG. 7).

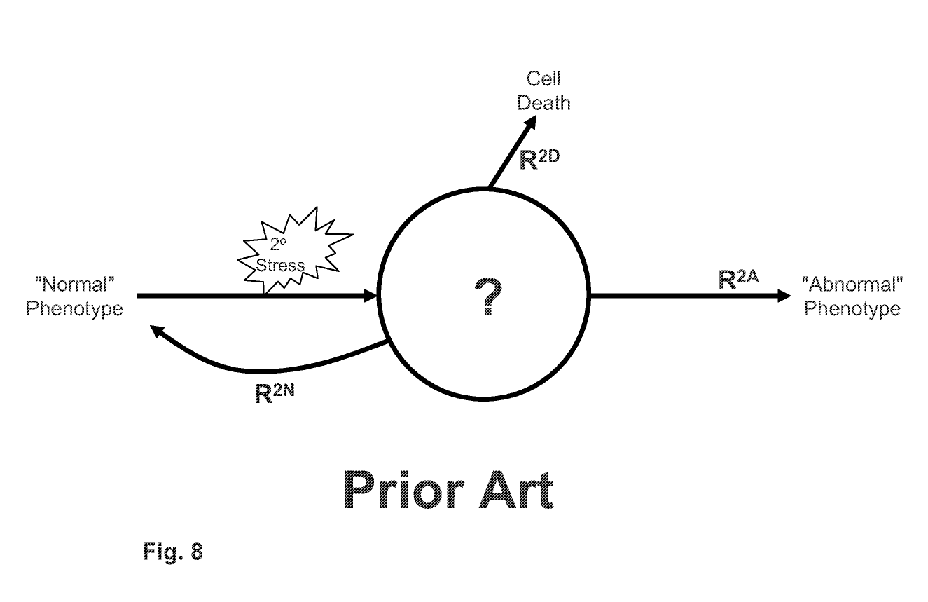

FIG. 8 is a diagram similar to that in FIG. 7, except that the cells are exposed to a different stressor, and exhibit the outcomes at different rates or proportions (e.g., factors R^{2N}, R^{2A}, and R^{2D}, respectively, in Figure).

Each of FIGS. 7 and 8 illustrate how toxicology testing is typically performed. Cells are exposed to a single stressor, and outcomes are assessed substantially without regard to the pathway(s) by which cells may have arrived at those outcomes. For example, if the “abnormal” phenotype (per FIGS. 7 and 8) that is being assessed in a toxicology assay is transformation of a normal cell into a tumor cell, then normal cells are exposed to a first stressor a selected concentration of a suspected carcinogen for a selected period of time), and the number of transformed cells are enumerated, either over time or after a selected period of time has elapsed.

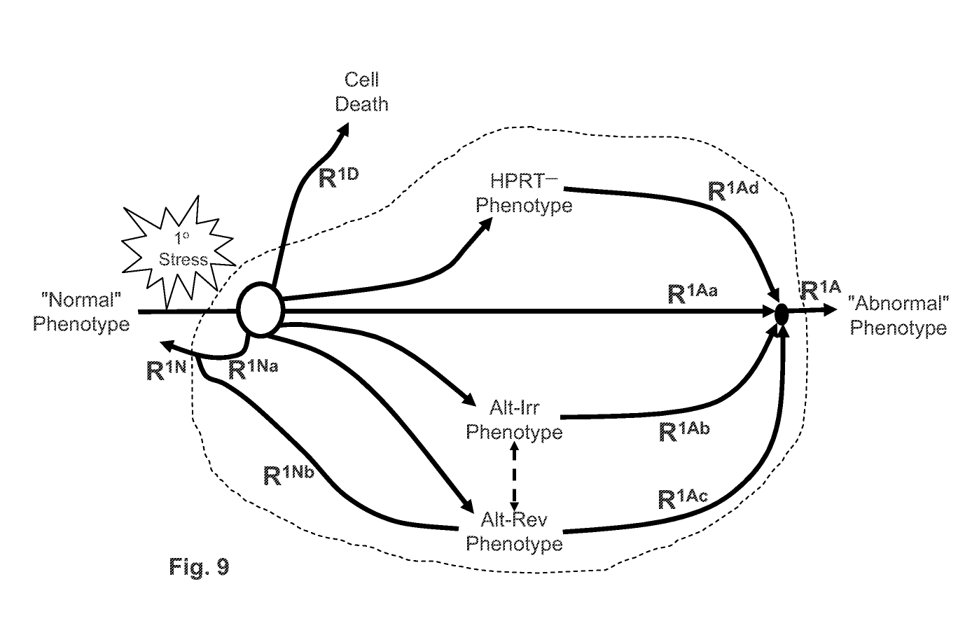

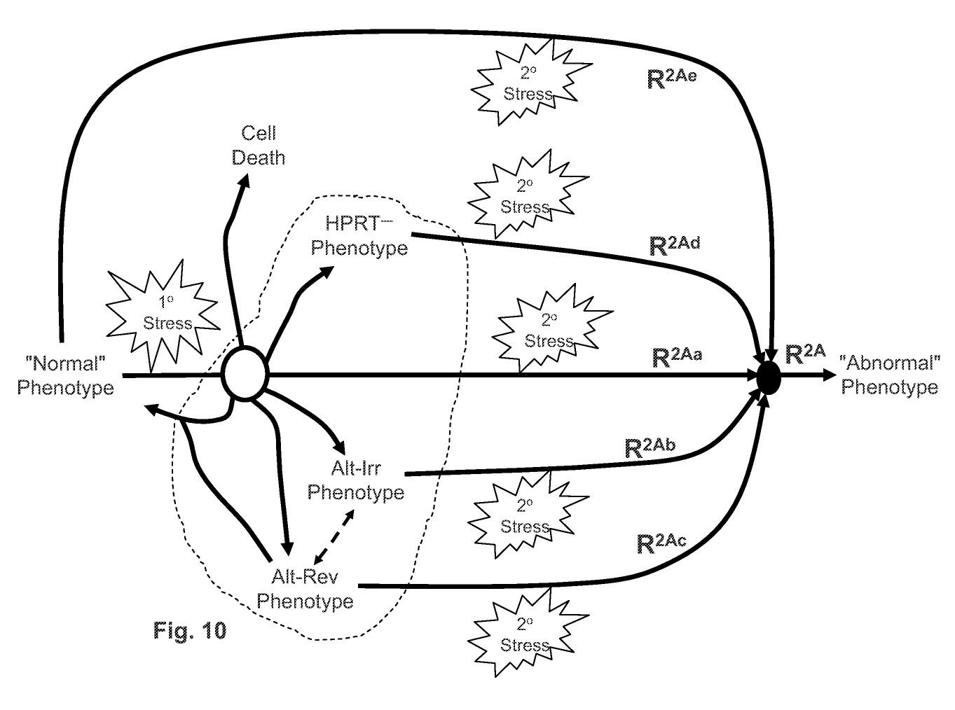

FIG. 9 illustrates some of the findings that have been made and that are described herein. When “normal” cells are exposed to a stressor, they may assume any of a number of phenotypes, of which five are illustrated in FIG. 9 (“normal,” “HPRT⁻,” “Abnormal,” “Alt-Irr,” and “Alt-Rev” phenotypes). Cells which exhibit “HPRT⁻,” “Alt-Irr,” or “Alt-Rev” phenotypes following exposure to the stressor may not proceed to the “Abnormal” phenotype, or may do so only at low rates or in low proportions (R^{1Ab}, R^{1Ac}, and R^{1Ad}) relative to the rate/proportion (R^{1Aa}) of “normal” cells that are directly changed to “Abnormal” cells upon such exposure. These findings may have relatively little significance for assessment of a single stressor. However, as shown in FIG. 10, the significance of the various post-stressor phenotypes can be much greater when those cells are exposed to a second stressor, because the various phenotypes shown in FIG. 9 can exhibit significantly different stress responses.

FIG. 10 incorporates the concepts illustrated in FIG. 9 (i.e., differentiation of “normal” cells into four or more other phenotypes after exposure to a first stressor), and illustrates the significance of cell differentiation on exposure to a second stressor. As in FIG. 9, some “normal” cells exposed to the second stressor will be converted to the “Abnormal” phenotype (at a rate or proportion of R^{2Ae} in FIG. 9; ignoring conversion of “normal” cells to other phenotypes in order to simplify the figure). Similarly, cells which exhibit “HPRT⁻,” “Alt-Irr,” and “Alt-Rev” phenotypes as a result of exposure to the first stressor can also be converted to “Abnormal” cells (at rates/proportions R^{2Ad}, R^{2Ab}, and R^{2Ac} respectively). Furthermore, some cells which were “Abnormal” following exposure to the first stressor can be altered to other phenotypes (rate/proportion opposite to R^{2Aa}) upon exposure to the second stressor.

Simply stated, FIG. 10 illustrates that conversion of “normal” cells to “Abnormal” cells upon exposure to a second stressor (rate/proportion R^{2Ae}) can be significantly different than the same conversion if the “normal” cells were exposed to a first stressor prior to exposure to the second stressor (rate/proportion is a function of R^{2Aa}, R^{2Ab}, R^{2Ac}, and R^{2Ad}). Put another way, exposure to a first stressor can significantly influence the ability of a second stressor to cause cells to assume a different phenotype.

FIG. 10 is, in fact, greatly simplified, in that it illustrates only transformation of cells into “Abnormal” phenotype cells upon exposure to the second stressor. The figure does not attempt to illustrate the ability of the second stressor to cause cells of the five phenotypes which experience the second stressor to transform into cells of phenotypes other than “Abnormal” or to die. Despite this simplification, the conclusion remains unchanged: the response of cells to a second stressor can depend significantly to the phenotype-differentiating response of the cells to a (prior) first stressor.

As an example, consider a second stressor that converts cells which exhibit the HPRT⁻ phenotype to “Abnormal” cells at a much higher rate (or at a much lower stressor concentration, or in a much higher proportion regardless or rate) than cells which exhibit the “normal,” “Alt-Irr,” or “Alt-Rev” phenotypes. Exposing “normal” cells will cause relatively few cells to convert to the “Abnormal” phenotype. However, if the “normal” cells are first contacted with a stressor that causes a significant portion of the cells to assume the HPRT⁻ phenotype, then exposure of the cell population to the second stressor will result in far more “Abnormal” cells being formed than would exposure of the original cell population to the second stressor.

These observations and findings have great significance in the field of toxicological and other cell screening methods. It was previously understood that cells used in such tests should be as homogenous as possible. However, what was not previously well understood was that exposure to a first stressor (even one which does not cause transformation of cells into a phenotype of interest) can greatly influence the susceptibility of cells to transformation into cells of the phenotype of interest by a second stressor. Thus, for instance, the methods and kits described herein permit skilled artisans to examine the effects of combinations of agents upon induction of disease and other pathological states. By way of example, the kits and methods described herein can be used to assess the effects of combinations of drugs on cells, including when the drugs are administered at discrete, different time periods. Similarly, the kits and methods can be used to assess interactions between drugs and environmental stresses (e.g., sunlight, radiation exposure, or heat) or between drugs and foods.

The balance of this disclosure describes assays and methods for usefully employing the observations and findings described above in summary form.

The present disclosure pertains to methods for identifying and quantifying the expression and functionality of outcomes of cellular stress response resolution pathways. These pathways include protective, damage avoidance stress resolution pathways, which do not result in a significant increase in permanent damage in DNA, the epigenome, or other cellular components, and damage tolerance stress response resolution pathways, which do result in significant changes in the amount and nature of reversible and/or permanent damage in DNA, the epigenome, and/or other cellular components.

The assay described in the present disclosure is applicable to the testing of chemicals, compounds, drugs, molecules, compounds, hormones, enzymes, physical agents, or conditions (agents or conditions) for their effects upon the expression and functionality of these pathways using human and animals cells cultured in vitro.

The ability to qualify and quantify the induction of cellular damage resulting from expression of stress resolution pathways in a variety of different human and animal cell types including but not limited to epithelial cells is useful for the discovery and identification of agents or conditions that induce or prevent or protect against stress resolution processes that result in an increase in the cellular damage burden. The information obtained from application of this assay will aid in the determination of risk to human beings from exposure to agents or conditions that induce stress responses and/or damage, and will aid in the discovery and development of drugs/agents/conditions that protect cells and/or prevent or reduce expression of damage tolerance pro-survival stress response pathways. In addition, information gained from the application of this assay can be used to identify agents and/or conditions that prevent or reduce pro-survival pathway expression in damaged, abnormal, or diseased cells, such as cells expressing a mutator phenotype or cancer cells, so that those cells can be removed more efficiently from humans or animals.

Several conditions and assumptions must be met to be able to analyze SR assay derived data with confidence and to be able to apply the formulas used to calculate the cloning effiencies (CEs) that are a quantitative endpoint determined in the assay. These assumptions, conditions and/or hypotheses are the same as those used in the T-cell cloning (HPRT) assay, the Tk assay, the PIG-A assay, and other gene locus-specific mutation assays that have been reported in the literature. These assumptions, conditions, and/or hypotheses conform to Poisson distribution principles, theory and statistics, and are incorporated herein (Haynes 1989).

The assay described in the present disclosure (termed the stress response or SR assay) pertains to the use of an exogenously-induced cellular stress response (termed the calibrated stress conditions exposure step of the assay) and associated methods to determine, qualitatively and quantitatively, the effects of exogenous exposure(s) to chemicals, drugs, physical agents and/or conditions, including sham exposures (i.e., no exposure), upon the expression, functionality, and outcome of stress resolution pathways in a variety of human and animal cells for the purpose of investigating basic cell and molecular biology, drug or agent discovery, drug or agent development, toxicity safety testing, and toxicity risk assessment testing.

The assay is applicable human and animal cells include the following: epithelial cells from any organ or tissue, glial cells of any type, neurons of any type, cardiomyocytes, cells of the immune system, including but not limited to dendritic cells, stromal cells of any type, fibrocytes and fibroblasts, stem cells including but not limited to embryonic stem cells, and endothelial cells from any organ or tissue.

Stress environment-inducing agents or conditions include but are not limited to agents that induce the types of stress shown in Table 4.

The SR assay is informative for beneficial or adverse effects upon stress response resolution pathways resulting from exogenous exposure of a given cell population to a test agent or condition. Stress response resolution pathways include but are not limited to pro-survival pathways that operate in a predominantly damage avoidance manner, and pro-survival pathways that operate in a predominantly damage tolerance manner, with or without induction of mutations in DNA and/or induction of alterations or modifications in the epigenome or chromatin. The SR assay assesses in a quantitative manner the expression and functionality of specific cellular pathways and changes in expression of those pathways while also assessing changes in damages levels and damage types at specific gene loci and in the epigenome in cells and cell populations.

The present disclosure pertains to the use of an exogenously-induced stress response for identification of, testing of and/or development of chemicals, drugs, physical agents and/or conditions that enhance or diminish the expression, functionality and/or outcome of cellular pro-survival pathways that operate, at the level of the individual cell or the cell population, in a predominantly damage avoidance manner and/or have the capacity to reduce the residual damage burden of DNA, the epigenome, or both.

Cellular Stress and/or Toxicity Paradigms

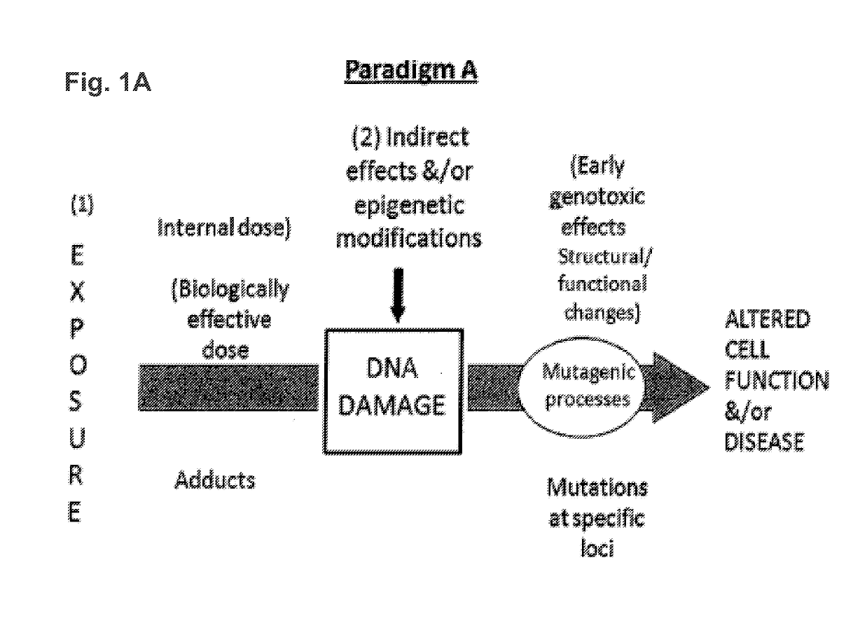

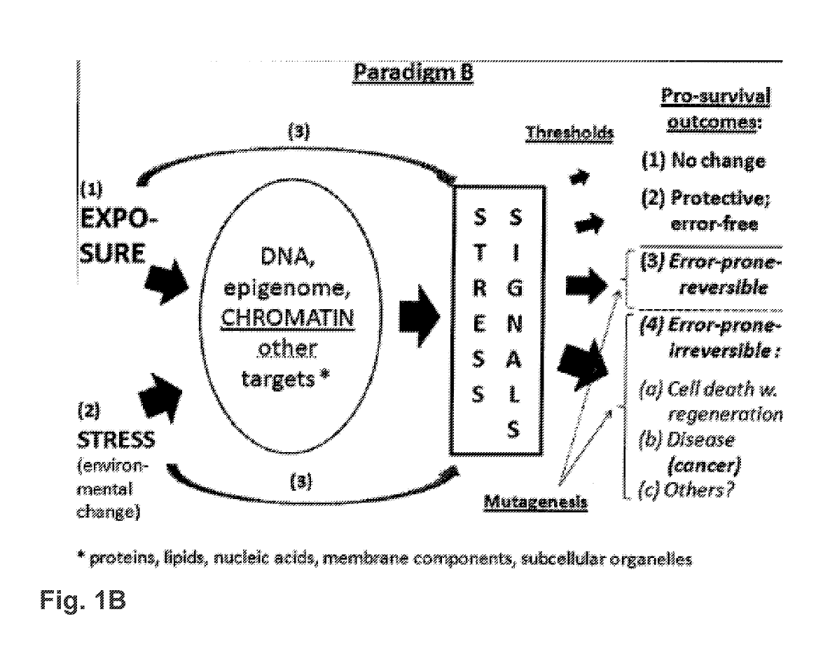

Currently there are two general paradigms or models used to understand the factors and conditions that lead to mutagenesis, altered cell function, and/or disease in life forms ranging in complexity from prokaryotes to complex metazoans like humans. The paradigm that summarizes the current understanding for complex metazoans is presented in FIG. 1A. This model allows for two pathways that lead to mutagenesis and altered cell function (see numbers (1) and (2) in FIG. 1A); one pathway involves direct damage to DNA, and the other pathway models indirect damage to DNA that is postulated to result from effects upon the epigenome or upon cell processes such as reactive oxygen species production. Both of these paths currently are hypothesized to pass through DNA damage, either directly or indirectly. In this model, mutagenesis and altered cell function occur as a consequence of this induced DNA damage, and is presented as an unavoidable result. Cell death or altered cell function also is an inevitable outcome of the events that set these modeled processes in motion. In spite of key limitations in this paradigm (see below), this paradigm currently is accepted as accurate and predictive of outcomes by the large majority of researchers working in the fields of toxicology, safety and risk assessment, and drug discovery and development.

The above model also was used in the past by microbiologists who studied prokaryotes and simple eukaryotes such as bacteria and yeast. Their research was aimed at understanding the factors and mechanisms leading to microbial mutagenesis that conferred antibiotic resistance, increased virulence, and/or a pro-survival advantage to a subset of the microbial cell population. Recent advancements demonstrated that the basic elements depicted in paradigm A were more complex than previously recognized; as a result of these research efforts, the model in FIG. 1A has been adapted and altered significantly to accommodate new information (see FIG. 1B). This new paradigm (paradigm B) includes the pathways and processes postulated in paradigm A for agents/conditions that damage DNA and induce high level stress signaling, but also represents a significant departure.

In paradigm B, three initiating or inducing pathways are postulated to be of importance in mutagenesis (see numbers (1), (2), and (3) in FIG. 1B), in brief, exogenous exposures to agents or conditions have been found in prokaryotes and simple eukaryotes to affect one or more of multiple cellular targets, thereby, affecting cellular elements critical to cell recognition and signaling of environmental change or stress. In other words, exogenous exposures (see number (I) in FIG. 1B) alter cellular stress signaling and response pathways in ways that are similar to those described for naturally-occurring environmental changes (see number (2) in FIG. 1B), and engage expression of cellular stress response pathways. These pathways are linked to a variety of cellular sub-components, and some investigators have hypothesized that all cellular sub-components have the capacity to recognize and to respond to stress. In the next step in the paradigm, these events result in intra- and inter-cellular stress signals that then serve as regulators of the cellular stress response. The forwards and backwards arrows indicate cross-talk between stress signals and chromatin as part of the regulation of the response. Recognition of the role of signaling leads to a subsequent recognition that exogenous agents that can act as stress signaling agents or signal-mimetic agents (see number (3) in FIG. 1B) can induce significant changes in cells without passing through a step in which cellular sub-components are changed or damaged. This recognition is critical for toxicity studies because it suggests novel mechanisms by which adverse agents can induce effects upon cells.

This new paradigm illustrates four postulated threshold-dependent pro-survival outcomes. These outcomes and their features include: (1) a role for signaling thresholds in outcome regulation, (2) the existence of low dose (low level signaling) responses not considered in paradigm A and that may have benign and/or beneficial effects, and (3) outcomes that are associated with reversible or (4) irreversible alterations in the genome, including but not limited to mutagenic processes. This new paradigm now is recognized and accepted, to varying degrees, by microbiologists working in the field of microbial genetics and mutagenesis. While a few components of paradigm B are recognized by some investigators working in the fields of mammalian cell mutagenesis and altered cell function, the whole paradigm is poorly recognized and not utilized by the large majority of scientists working in the fields of genetics, epigenetics, mutagenesis, toxicology, and/or safety and risk assessment in higher eukaryotes.

It is believed that the outcomes shown in paradigm B are influenced by a range of factors that are too numerous to include in the figure; in general, outcomes of stress responses or stress signals are influenced by cell type, tissue and organ type, sex, age, strain (genetic polymorphisms), species, and the amount and nature of the residual damage burden in affected cells. The range of response resolution outcomes includes both cell death and cell survival responses; the latter are described in more detail in Table 1.

The above two paradigms are presented and discussed because the results of the SR assay cannot be understood or interpreted using paradigm A, but instead are compatible with the steps and processes depicted in paradigm B. This is surprising since, to date, there is a paucity of evidence for the pathways and outcomes depicted in paradigm B in the cells of complex metazoans such as human beings. This paucity largely is due to the fact that assays available for the study of human cellular responses to stress in a variety of model systems and cell types have been lacking, with the result that models were based upon studies of lower organisms (yeast, plants, flies, worms, frogs, fish, and mice are the seven most common models (Kloc 2008)), and studies of mutagenic and epigenetic-altering events largely were confined to peripheral blood cells of rodents.

The SR assay provides a significant departure from this earlier model and offers the opportunity to develop an improved understanding of factors that affect stress sensing/signaling pathways, pathways involved in stress response resolution and pathways involved in ECM.

Cellular Stress Responses and Their Resolution Pathways

To maintain optimal cell function and to limit disease occurrence, it is necessary to be able to detect and to quantify the ability of human and animal cells to resolve stress. This need includes being able to identify and measure the effects of environmental agents or conditions, including exposures to exogenous agents, upon cell function, including stress response function. These effects include but are not limited to changes in cell genotype and phenotype, the latter being determined by the epigenome. Several fields of investigation have developed and been pursued as part of the effort to understand the causes, mechanisms, and prevention of deleterious changes in cellular behavior and function; the results of this work have applications to diverse fields including basic biology and mechanisms of disease pathogenesis, toxicology and risk/safety assessment, drug discovery and development, and prevention and treatment of human diseases. A subset of these studies has focused upon the investigation of mutagenesis, i.e. the processes that result in the formation of persistent alterations in the DNA base code.

A variety of in vitro and in vivo models have been developed to aid investigations in this field. More recently, a group of studies have found that epigenetic modifications, i.e. modifications to DNA-associated proteins and accessory factors, also have profound effects upon cell behavior and function, highlighting the need to be able to detect and quantify epigenetic modifications.

Another line of research has focused upon the investigation of the genetic pathways that are used by cells to respond to and to resolve environmental changes, including stress-inducing environments, and these investigations have found links between cellular stress response resolution pathways, mutagenesis and epigenetic alterations, and permanent changes in cell behavior and function.

Stress is considered to be a change or condition perceived by a cell or cell population as resulting in stress signaling and (usually) as sufficiently adverse to alter the cell's homeostasis and/or requiring an adaptive response. This adaptive response is mediated through a variety of changes, including but not limited to, changes in gene expression, up- or down regulation of existing cell processes, and changes in the epigenome. The purpose of this response is to protect the cell from damage, to repair induced damage, to restore homeostasis, and to enhance cell survival during and after stress.

Stress usually is induced by an environmental change and can be induced by exposure to an exogenous agent, such as a toxin. Due to fluctuations in environmental conditions, stress is a phenomenon encountered by the cells of all life forms, thus, it cannot be avoided and no non-diseased, viable cells have been identified that lack the capability to respond to stress.

Stress responses are categorized loosely as ‘general’, as seen in bacteria and simple eukaryotes, or ‘specific,’ as seen in higher eukaryotes (Murray et al, 2004). Stress responses in animals and humans are cell type-, organ- or tissue type-, sex-, strain-/species-, and age-specific. They probably also are affected by heritable gene polymorphisms found within groups of closely-related humans or animals. They operate across a range of levels and are threshold sensitive. As a result, cellular stress responses are linked to the levels and/or amounts (including cumulative amounts) of the agent or condition that induced or caused the stress environment. Initiation, progression, and resolution or stop phases have been recognized for stress responses. It is believed that these phases are controlled by different groups or families of genes.

Responses to stress may be adaptive or maladaptive (i.e., in the case of cell dysfunction or disease), but serve the purpose of enhancing cell survival and eliminating damaged cells in the face of environmental change. They have been the subject of much study in the past decade because they are hypothesized to constitute the mechanisms for adaptation to environmental change, for successful, predominantly error-free damage resolution, and to provide the means for survival under adverse conditions, including life-threatening conditions.

There are multiple specific stress responses described in complex metazoans such as human beings, and there is evidence that signaling between cellular organelles under stress may occur, thereby providing potential links between specific types of stress responses. There is some evidence that one specific stress response can induce expression of other specific stress responses, and/or one type of stress response may induce expression of cellular pathways associated with other specific types of stress responses and their resolution pathways.

Stress responses are regulated by signaling. Some environmental factors, such as pressure or oxygen levels, can serve as signals. Cells also have mechanisms for sensing the environment and transmitting signals within the cell and to neighboring cells. Stressed cells also signal to surrounding cells. Thus, a few stressed cells can induce stress responses across a radius of neighboring cells (bystander effect) (Belyakov et al., 2002).

It is believed that the nature of the inducing agent and signaling type, intensity, and location regulate the stress response type and level. These features link stress responses to thresholds and result in cell-population-based (rather than individual-cell-based) effects.

A full description of the molecules, complexes and/or changes involved in signaling is too long to include here. The number of these also is expanding as new elements are recognized and described. Future work is expected to reveal additional signaling elements.

DNA damage serves as one type of signal to induce a stress response in cells; Bartek and colleagues (2007b) found that replication blocking lesions were the type of DNA lesion most frequently associated with signaling of stress. Early stress induction events and the associated functional factors include ATM, ATR, Ku70/80, Artemis, DNA-PK, RNF8, RNF168, (Morio and Kim 2008, Panier and Durocher 2009).

Examples of molecules, complexes, and/or changes recognized to have a role in various types of stress signaling include checkpoint-associated elements such as ATM-Ch2, ATR-Chk1, POL32 (Bartek et al. 2007a, Peng et al. 2007), Chk2 (Li and Stern 2005), DNA repair-associated elements (Hoogstraten et al. 2008, Soutoglou and Misteli 2008), elements involved in chromatin remodeling including ATP-dependent chromatin remodelers pang et al. 2009, Polo and Almouxni 2007), elements involved in signal amplification (Karagiannis and El-Osta 2004, Schreiber and Bernstein 2002), and elements involved in signal sensing and responding to signals (Bonatto 2007).

Elements involved in the switch from expression of predominantly error-free (damage avoidance) to error-prone (damage tolerance) stress resolution pathways in microbes include I-Sce endonuclease, double strand break repair proteins, DinB-error-prone polymerase, and RpoS (Ponder et al. 2005). Other elements include products of the umuD gene, RecA recombinase, and replication machinery components (Simon et al, 2008) and components of the SOS system (Delmas and Matic 2006). Elements shown to have roles in human cells include Rad18 (Nakajima et al. 2006), poly(ADP-ribose) polymerase-1 and -2 and poly ADP-ribose (Dantzer et al. 2006), PCNA (Langie et al. 2007, reviewed in Simmons et al. 2009).

It is believed that chromatin and its components represent a central point linking environmental changes, signals, and cellular responses leading to stress response induction, progression, and resolution. Chromatin has been recognized to have roles in cell replication, transcription, recombination, and DNA repair. Chromatin also has roles in stress response expression including receiving and sending signals, regulating stress response outcomes, and maintaining ECM. The ability of chromatin to undergo reversible adaptive changes to the environment is recognized as an essential feature of cellular stress responses in human and animal cells (Soutoglou and Misteli 2008, Yeung and Durocher 2008).

Once a stress environment has been signaled and a response induced, that response must be resolved in order for cells to survive and return to baseline function (Harper and Elledge 2007, Murray, et al. 2004). The resolution of the DNA damage response (DDR) and some other stress responses currently is recognized to have three major outcomes: (i) cell-cycle checkpoint induction, (ii) DNA repair and survival, and/or (iii) cell death by apoptosis, or by other cell death-associated pathways, including senescence.

The SR assay identifies and quantifies the expression and functionality of cellular pathways that enhance cell survival during and after exposure to a stress environment. These pathways are referred to as pro-survival pathways.

Studies of prokaryotes and simple eukaryotes support the conclusion that there are three major pro-survival pathways, or groups of pathways (four, if the ‘no change’ response is included), that cells can employ to resolve stress-associated damage that exceeds a threshold, including but not limited to DNA damage, and survive (see Table 1).

The first major pro-survival pathway (referred to here as cell protective, predominantly error-free damage resolution pathways; damage avoidance pathways) is characterized by up-regulation of DNA repair and apoptosis pathways. These pathways may also include changes in the production of protective substances, metabolic changes, and other changes in cell processes (see Eller et al. 1997 as an example). Expression of this response allows cells to resolve a stress challenge with no detectable increase in residual damage burden in DNA or chromatin. Damage avoidance paths are hypothesized by some investigators to have the capacity to resolve or eliminate induced and/or pre-existing lesions, and thus, potentially to have beneficial effects. It is believed that assays capable of detecting and quantifying this response and demonstrating its protective, error-free, pro-survival capacity in human and animal cells have not been available prior to the development of the SR assay.

The second set of pro-survival pathways involves the expression of damage tolerance processes, i.e. resolution pathways that result in a net increase in residual DNA and/or epigenome damage burden. In prokaryotes, these resolution pathways result primarily in reversible (from the point of view of the cell population) mutations or alterations. These changes include chromatin modifications and/or epigenetic alterations described in higher eukaryotes. To distinguish this group of damage tolerance pathways from those described below, these pathways are referred to here as damage tolerance pathways (damage-tol paths).

The last major group of pro-survival paths is referred to here as the damage tolerance-irreversible pathways (damage tol-irr paths). This group of pathways is characterized by down-regulation of DNA repair processes and of apoptosis and by expression of a mutator phenotype. They also are characterized by permanent DNA and/or epigenetic modifications and significant, irreversible phenotypic alterations, as are seen in cancer and several other human diseases.

Mutagenesis associated with expression of both of the above damage avoidance pathways has been recognized for several decades and is referred to as stress-induced mutagenesis, untargeted (non-targeted) mutagenesis, or environmental mutagenesis. The terms stationary phase mutagenesis, hypermutation, genomic instability, and mutator phenotype also are applied to subpathways and/or to manifestations of these damage tolerance resolution pathways.

Evidence for the expression of all three of the above groups of stress response resolution pathways has been found in prokaryotes, simple eukaryotes, and a few animal species including humans (see Eller et al. 1997). However, It is believed that (i) the relationships between these pathways are poorly understood, (ii) quantitative assays for assessment of the expression and functionality of these pathways in human and animal cells are lacking, and (iii) these pathways have not been evaluated simultaneously in a single model system as now has been done using the SR assay. Thus, SR assay data are some of the few data informative for the relationships between these pathways and for the effects of exposure to a test agent or condition upon pathway outcomes in complex metazoans such as mice and humans.

Evidence from a few model systems, when combined with the results of the SR assay, supports the conclusion that cells follow a hierarchy in expression of pro-survival pathways used to resolve a stress environment. Damage avoidance paths are expressed first and serve to counterbalance the damage tolerance pathways, thereby protecting the cell population from the deleterious effects associated with expression of these latter pathways in response to stress. There also is evidence that expression of the damage avoidance paths represses or reduces expression of the damage tolerance pathways, so damage avoidance paths may be expressed in the absence of expression of damage tolerance paths.

It is believed that cells under genotoxic stress first will express damage avoidance pathways; if these pathways are unable to resolve the stress environment, then cells will progress to expression of the damage tol-rev pathways, and will use the damage tol-irr paths only if the previous two pathways fail and/or the stress environment continues.

In a cell population, one or more paths may be expressed simultaneously but by different cells. The possibility that cells can express more than one path at the same time cannot be ruled out. This hierarchy in expression of SR resolution paths is compatible with existing theories of cellular transformation, including the hypotheses of Loeb and colleagues (2001, 2003) that transient or persistent genomic instability is necessary for neoplastic transformation to occur. However, the complete scenario and its associated hypotheses, is novel.

The above hierarchy reflects the signaling nature and/or intensity of the stress response-inducing agent or condition. It is believed that stress response induction can occur at four or more levels: (i) a no-change (no-response) level, where lesion-associated signal intensity is too low to induce detectable changes in gene expression or other markers of response; (ii) low to moderate signaling levels, usually resulting from an acute and/or very low level exposure to stress; (iii) moderate signaling levels resulting from a moderate stress environment of limited duration; and (iv) high signaling levels resulting from a high level and/or prolonged, or chronic stress environment.

Short term, moderate stress signaling is expected to be resolved using primarily damage avoidance and damage tol-rev paths, and pathway choice will be influenced by the cell type involved. However, if the signaling intensity and/or duration pass a threshold that is cell type specific so that the signaling intensity is perceived by the cell as being prolonged, chronic, and/or of high intensity, it is believed that the outcome is mediated primarily by damage tol-irr paths. Because stress responses vary between cell types, tissue types, organs, sexes, strains, species, ages and genetic background, it is not possible to define one threshold for all cells at which the signal intensity of a stress environment results in a switch from utilization of short-term moderate intensity resolution paths (i.e., damage avoidance and damage tol-rev paths) to damage tol-irr paths that are associated with chronic or high-intensity stress.

To date, agents that induce a DNA damage response(s) or genotoxic stress response(s) are the types of agents that have been used most frequently as stress-inducing agents in the calibrated stress response induction/conditions step of the SR assay. The use of these agents to induce a moderate, short-term stress environment made it possible to evaluate all three of the major stress resolution paths described above. In addition, the assay has been used primarily to evaluate the effects of pre-exposures to DNA damaging agents.

Resolution of DNA damage/genotoxic stress involves a variety of inter-related mechanisms that act in a coordinated fashion and that include cell cycle regulation, repair pathways, many aspects of cellular metabolism, and cell death (Barzilai et al, 2008). Thus, induction of a DNA damage stress response during the calibrated stress conditions step of the SR assay allows assessment of the functionality of these mechanisms. However, the SR assay also can be used to evaluate stress resolution outcomes following exposure to any level of stress response induction in the calibrated stress conditions step of the assay, including but not limited to a chronic, high intensity stress challenge (see example 8 below).

Agents that induce a DNA damage or genotoxic stress response in the calibrated stress conditions step of the SR assay were chosen initially for use because this type of stress response has been the subject of much recent research, its resolution is associated with cellular avoidance or tolerance of mutations and the eventual development of diseases with a mutation-associated pathogenesis.

It is believed that several specific stress responses utilize the above described pathways, which currently are associated with DNA damage response resolution, during the process of resolving or adapting to stress. These stress responses include cytotoxic stress, glucose-, amino acid- and/or nucleotide-deprivation stress (nutrient deprivation stress), hypoxic stress, oxidant or oxidative stress, temperature (heat or cold) stress, and others. As a result, mutagenesis can be observed in cells adapting to stress conditions that are not known to directly damage DNA.

The SR assay described herein is capable of evaluating the expression and functionality of stress resolution outcomes following induction of a variety of specific stress responses, not just the DNA damage/genotoxic stress response.

Stress Resolution Pathway Outcomes

To understand the results of the SR assay and the information provided by those results, it is necessary to describe the above stress resolution pathways in greater detail.

The damage avoidance paths are defined here as stress resolution pathways that resolve stress environments and/or induced damage with little to no increase in residual DNA or epigenetic damage burden and retention of high survival capacity (relative to damage tolerance pathways). The damage avoidance paths appear to be the earliest reported of the above described stress resolution pathways (Hardy 1892), but they also are the most controversial. Some investigators deny their existence and the validity of any results supporting their expression. This controversy exists for at least two reasons. First, in spite of circumstantial evidence of their existence and functionality, few to no methods of directly demonstrating damage avoidance pathway functionality have been described prior to the development of the SR assay. Second, it is believed that damage avoidance paths have the capacity to reduce the residual damage burden, at least under some circumstances, but identification of mechanisms responsible for this effect have been lacking. In the face of the absence of explanatory mechanisms and direct evidence of pathway functionality, investigators have challenged the validity of the supporting data. A subset of literature reports reflect the assumption that the DNA damage response can be resolved only in a damage tolerance or accumulation manner. This assumption further advances the viewpoint that damage avoidance stress response and/or damage resolution pathways do not exist or have very weak resolution capacity.

It is believed that the SR assay described herein is the first in vitro cell culture assay with the ability to detect and quantify expression of this pathway and its damage avoidance resolution capacity in a variety of cell types including but not limited to human epithelial cells, which are the cell types most prone to neoplastic transformation.

Damage avoidance paths have been studied in yeast and bacterial models; few examples have been reported in humans or other mammals (see Eller et al. 1997 for an exception to this trend). At least two genes/gene families have been described with potential roles in damage avoidance pathways and for which supporting evidence is available. The first consists of the p53 gene, its co-factors, accessory elements, target pathways and subpathways. The second group is the NT-kappaB family, its co-factors and accessory elements, target pathways and subpathways. NF-kappaB is recognized as a key mediator of pro-survival pathways with a recognized role in damage tolerance stress response resolution. The role of NF-kappaB in damage avoidance paths is hypothesized but little is known about its function under these conditions. A wide range of other cell processes and/or pathways have been shown to function in cell survival and may function in damage avoidance pathways (see Table 2).

Recent research also has found evidence in a yeast model that DNA damage repair and signaling elements have roles in damage avoidance pathways functionality, even in cases where the cell is resolving lesions for which the DNA repair elements have no role in lesion repair. These investigators found that elements of homologous recombination, post-replication repair, DNA helicases, chromatin maintenance factors, and DNA damage signaling proteins had a significant role in protecting cells from MNNG (N-methyl-N′-nitro-N-nitrosoguanidine) cytotoxicity (Cejka and Jiricny 2008). Thus, new information about damage avoidance pathway-associated factors is becoming available rapidly, and additional factors/elements are expected to be identified in the future.

In addition to specific pathways or factors with cell protective function, a wide variety of cell functions have been described as contributing to damage avoidance stress response resolution under specific circumstances and in specific models. Table 2 presents a summary list of the cell functions/systems/processes that have been cited in the literature as contributing to cell protection and damage avoidance stress resolution. Evaluation of the degree to which each process contributes to damage avoidance paths in human cells of a specific type under specific stress conditions remains to be determined.

Of the cellular processes presented in Table 2, DNA repair has received the most study for its potential to resolve DNA damage in an error-free manner, but only recently has evidence been presented showing that essentially all known DNA repair systems have both error-free and error-prone repair capacity. A summary of major DNA repair pathways and some of the functional elements involved in both error-free and error-prone lesion repair are presented in Table 3.

The other two groups of pathways used to resolve a stress challenge are termed here damage tolerance pathways. These pathways differ from damage avoidance paths in that their existence is not considered to be controversial, but their role in stress response resolution in mammals is not well understood and most information still is derived from bacterial and yeast models. In addition, the distinction between the two major sub-pathways (described below) that make up these pathways is based in part upon SR assay results and is recognized inconsistently in the literature. Expression of damage tolerance pathways is referred to by several alternative names including stress-induced mutagenesis, adaptive mutagenesis, and an early name was environmental mutagenesis. In addition, the term untargeted (or non-targeted) mutagenesis is used to refer to these pathways by some investigators, while these terms are used by others to refer to the damage tolerance pathways associated with genomic instability, resulting in ambiguity in the literature. For discussion purposes here, damage tolerance stress response resolution pathways are referred to as either damage tolerance-reversible (damage tol-rev paths) or damage tolerance irreversible (damage tol-irr paths). Differences between these pathways are presented below.

In bacteria and yeast models, expression of damage tol-rev paths is characterized by induction of primarily reversible genetic and/or epigenetic alterations. Comparable changes in higher eukaryotes include epigenetic alterations/modifications, amplification (at the protein, mRNA or (less often) gene level), paramutations, and possibly stationary phase mutagenesis. Epigenetic processes include DNA methylation, histone modifications, nucleosome repositioning, higher order chromatin remodeling, non-coding RNA patterns, and RNA and DNA editing, and changes in microRNA expression (Mehler 2008). Detection of alterations in DNA or chromatin due to expression of damage tol-rev paths can reflect changes in the expression and/or functionality of one or more of these processes.

Damage tol-rev paths also may include expression of processes involved in stationary phase mutagenesis. Due to ambiguity/uncertainty in the literature, it is not possible to determine with certainty which damage tol-rev pathways are associated with stationary phase mutagenesis. This type of mutagenesis is a cell-mediated, error-prone or mutagenic stress resolution process that also may be expressed as a subcomponent of damage-to-irr paths.

Stationary phase mutagenesis is expressed in cells that are stalled at a phase of the cell cycle as part of SR resolution. It results primarily in point mutations and +1/−1 frameshift mutations in bacterial and yeast models. Its occurrence has been hypothesized in human cells (Galhardo et al. 2009), and there is a small body of evidence suggesting the expression of stationary phase mutagenesis in human cancer cells (Gonzalez et al. 2008). It is believed that the SR assay described herein is the first assay applicable to human and other mammalian cells that can detect and quantify reversible genomic and phenotypic/chromatin alterations indicative of expression of damage to-rev pathways.

It is believed that damage tol-rev paths are mediated, at least in part, by increased expression of error-prone polymerases, error-prone DNA repair pathways, and increased translesion synthesis (TLS) expression, an error-prone subpathway of post-replication repair. During damage tol-rev pathway function, TLS is postulated to work primarily upon induced lesions. It is believed that damage tol-rev paths are not associated with expression of a mutator phenotype and loss of all constraints upon TLS pathway enzymes. It is also believed that transient, reversible phenotypic alterations, which are detected in one type of surviving group of cell clones in the SR assay, are a manifestation of transient changes in the epigenome and/or in chromatin conformation and/or structure and may reflect changes that have occurred primarily in histone proteins and that affect gene expression. Identification of surviving cell clones with these characteristics may reflect changes in the expression and/or functionality of cell systems that maintain the histone code and/or modify histone proteins.

It is believed that the SR assay described herein is the first assay to assess epigenetic alteration frequencies in a quantitative manner and to do so along with assessment of genetic mutation frequencies. The SR assay thereby provides a more complete assessment of damage tolerance pathway expression, functionality, and outcomes than prior assays. A few investigators have hypothesized that induction of reversible epigenetic alterations in cells may predispose those cells to the accumulation of permanent, irreversible mutations (Sawan et al, 2008, McKenna and Roberts 2009). The SR assay can be used to investigate this hypothesis.

Damage tol-irr pathways are distinguished from the two other groups of stress response-associated pro-survival pathways by the presence of damage throughout the genome (rather than only at specific sites) and evidence that a period of transient or persistent genomic instability has occurred. The same phenomenon that is being referred to here as expression of damage tol-irr pathways also may be referred to in the literature by the term ‘untargeted (nontargeted) mutagenesis’. Genomic instability or a mutator phenotype generally is considered to reflect aberrant cell function and may or may not be associated with stress response resolution. However, untargeted mutagenesis is recognized as a cell-mediated mutagenic process that is expressed under conditions of severe stress that threaten the cell population survival. Results obtained using the SR assay described herein support the conclusion that genomic instability, expression of a mutator phenotype, and expression of untargeted mutagenesis may be different names for the same, stress-associated, mutation-inducing phenomena.

Genomic instability, like untargeted mutagenesis, can occur in cells in which regulation of stress response resolution pathways has been damaged so that damage tol-irr paths are expressed inappropriately and/or persistently, the latter case reflecting damage to stress response stop mechanisms. However, damage tol-irr paths (genomic instability) also are expressed, and probably are expressed more frequently, as a stress environment-resolution process that is used to enhance cell survival under very adverse conditions. Although the outcome of damage tol-irr paths expression is viewed as deleterious, undesirable, and associated with disease occurrence, it is believed that these paths are part of all/most cells' stress response repertoire, and their expression is not necessarily indicative of abnormal cell function, but rather of expression of pro-survival pathways in the face of a very adverse stress challenge. It is believed that this understanding of the role damage tol-irr paths and genomic instability in human cells is novel, although it is accepted by scientists investigating bacterial and yeast models.

The name ‘untargeted’ (or ‘non-targeted’) stems from the Observation that the induced mutations are not formed solely at sites of DNA damage. Instead, mutations can be formed at undamaged (distant) sites, they can be induced in undamaged cells, and they may occur across cell generations or organism generations (bystander effects). Expression of this group of pro-survival pathways results in the loss of individual cells that form non-viable mutations, and the genomic integrity of surviving cells is compromised. Under conditions of severe stress, the resultant high mutant frequencies, which are induced across the entire genome in an apparently random fashion, enhance the survival of a subset of cells in the population because their mutations are advantageous, preventing complete loss of the population's genome.

The association of damage tol-irr paths with stress environments and their outcomes is compatible with classical Darwinian theory but is less compatible with neo-Darwinian or Lamarkian theory. The existence of cell-mediated processes characteristic of damage tol-irr paths expression has been hypothesized by several investigators in the past century, and these pathways have been studied using models from bacteria to humans. Damage tolerance stress resolution, including damage tol-irr pathway expression or untargeted mutagenesis now is hypothesized to be the major mechanism leading to antibiotic resistance in microbes, to chemotherapy resistance in cancer cells, and to have a role in cancer initiation, progression, and/or malignancy. These pathways also are considered to have roles in other human diseases and other pathological physiological states with hypothesized mutagenesis-associated pathogenic mechanisms.

Several changes in gene expression are associated with expression of damage tol-irr paths; these include reduction or suspension of DNA proofreading functions, down-regulation of DNA mismatch repair (and probably other DNA repair pathways) and down-regulation of intracellular and extracellular apoptosis. At the same time, damage tolerance SR resolution pathways are up-regulated, error-prone polymerase expression is increased, and constraints on TLS are removed or lost. It is believed that major changes in the epigenome also occur.

The genetics of stress-associated mutagenesis or untargeted mutagenesis have been described in microbes and a few human or other animal cells, primarily in the context of investigating untargeted mutagenesis. In E. coli, induction of a stress response also induces two mutagenic processes: (1) TLS, and (2) genomic instability that induces mutations at undamaged sites. Involved genes identified in E. coli include genes associated with DNA excision repair, RecA protein, single-strand binding protein, beta-sliding clamp, gamma-clamp loading complex, and DNA polymerases II, III, IV (DinB), and V (Tang et al, 1998, 2000, Zhu et al. 2003). Factors and elements involved in MS include Rev3 and Rev7 (the components of DNA polymerase zeta). Rev 1, and factors/elements involved in post-replication repair (see Table 3). Some pro-survival pathways such as those described above (p53 and NF-kappaB-mediated) also may function during expression of damage tolerance pathways; in this case pro survival pathway expression enhances the survival of cells with significant increases in residual damage burden, as opposed to enhancing the survival of cells with little to no change in residual damage burden.

The number of molecular elements that are involved in stress resolution pathways in one or more human cell types are too great to provide a complete list here. Some of the most studied elements and pathways include ATM/ATR-mediated pathways (Peng et al. 2007). Fanconi elements, MAPKs (mitogen activated protein kinases). Raf/Mek/ERK kinase cascade, PI-3K/AKT/mTOR, histone modifiers (Schreiber and Bernstein 2002.) XPA (xeroderma pigmentosum A protein) (Hoogstraten et al, 2008), retinoblastoma pathways (Bartek et al. 1997), and cell cycle control and checkpoint pathways (Bartek and Lucas 2007).

In addition, recent work has described gene elements involved in the switch of bacterial cells from expression of error-free DNA repair to mutagenic TLS under conditions of stress (Simon et al. 2008). In brief, umuD gene products in E. coli coordinate this switch; since stress response and resolution pathways are shared across phyla and species, these findings support the postulate that similar switches occur in human cells, although the processes and mechanisms are hypothesized to be more diverse and/or complex.

Checkpoint adaptation is a damage tolerance-associated process that is hypothesized to occur during cell senescence and that has been recognized in epithelial cells, but rare in fibroblasts. Thus, recent checkpoint adaptation may be a cell-type specific damage tolerance mechanism (Gosselin et al, 2009, Martien and Abbadie 2007). Senescence (permanent removal of cells from the growth cycle) is associated with aging and increased disease risk and is thought to be due to a combination of telomere shortening and oxidative damage Senescence also is hypothesized to represent one mechanism used by cell populations avoid the proliferation of damaged cells, thereby acting as a tumor suppressor or damage avoidance mechanism (Martien and Abbadie 2007). However, this latter role for senescence may not always be correct. It is believed that senescence has a role as a pro-tumor mechanism, through effects of oxidative damage upon DNA during senescence with potential targeting of oncogenes and/or tumor suppressor genes. During senescence, ROS accumulate and are hypothesized to serve as a mechanism for mutation induction that then leads to checkpoint adaptation (also called post-senescence emergence) (Gosselin et al. 2009). In epithelial cells senescence is driven to a greater extent by oxidative stress than by telomere shortening, and this may explain, at least in part, the cell type specific nature of checkpoint adaptation (Martien and Abbadie 2007).

The high mutant frequencies associated with the above stress resolution pathways have the potential to lead to irreversible changes in cellular function while also conferring a survival advantage upon the affected cells if the changes are advantageous in the adverse environment. Several systems vulnerable to mutational damage and known to be associated with human diseases include damage to the unfolded protein stress response machinery (endoplasmic reticulum SR), the stress resolution stop machinery (with the result that stress responses are expressed persistently, including persistent genomic instability, or a mutator phenotype), the cell-cell communications systems, the apoptotic machinery, and/or the ECM system. Dysfunction in several of these systems already has been associated with neoplasia; Loeb and colleagues (2003) have hypothesized that genomic instability is required for neoplastic transformation because it is the only process capable of inducing large scale genomic damage in the short time frame needed to ensure survival and a significant change in cell phenotype. A few investigators have hypothesized that permanent damage to the systems/processes that regulate and maintain ECM also must be induced for cellular transformation to occur (Bachman et al. 2006, Ting et al, 2006).

The SR assay detects high mutant frequencies consistent with genomic instability, and also detects and quantifies the survival frequency of cell clones with irreversible phenotypic alterations that serve as indicators of permanent alterations in ECM and/or systems that maintain and restore it. Thus, the SR assay can be used to determine the impact of exogenous exposures upon the functionality of these cell systems and processes. The SR assay's ability to assess reversible and irreversible epigenetic alterations and to quantify irreversible changes in cellular memory is novel.

Epigenetic Cellular Memory (Epigenetic Based Cell Memory, ECM

To understand the significance of the ability of the SR assay to quantify changes in ECM, and the effects of those changes, additional information is necessary. ECM and associated processes have been hypothesized to exist as part of the phenomenon referred to as cellular memory, with roles in the regulation of global patterns of gene expression and protection of the genome by silencing viruses and transposons (Eilertsen et al. 2007). Mechanisms involved in this type of memory include DNA methylation and histone modifications; histone modifications are hypothesized to be involved in transient cellular memory changes, while DNA methylation changes are hypothesized to be involved in persistent and/or irreversible changes. It is believed that the roles of these epigenetic changes in cell memory may have considerable overlap and a better understanding of their roles remains to be elucidated. Importantly, even though chromatin undergoes large changes during the course of cell growth, differentiation, replication, adaptation to stress challenges, etc., these modifications are reversible and ECM systems are hypothesized to be responsible for restoration of epigenetic patterns to their original, baseline status so that the parental cell phenotype is maintained and expressed. Little is known about these systems, but they are hypothesized to include the DNA methylation system and a number of other systems, processes, and/or elements (see Appendix BBB). ECM has been recognized to be inactivated in some diseases such as cancer, and this inactivation is hypothesized to be due to the accumulation of a mutational burden in critical components of the system. If this system is damaged, the major outcome is the loss of the cell's ability to restore epigenetic patterns, including chromatin conformation and/or structure, to that of the parental cell, and thus, to restore the cell to expression of its original phenotype. In such cases, stress-induced chromatin conformational changes are permanent and irreversible, leading to irreversible phenotypic alterations, an endpoint quantified in the SR assay. Mutational damage to this system could result from a period of transient or persistent genomic instability, which now is accepted by many investigators to be a prerequisite for neoplastic transformation.

ECM is hypothesized to be under the control of a macro-molecular complex referred to as the ‘epigenetic code replication machinery’; the complex is hypothesized to be composed of enzymes involved in DNA methylation and histone modifications such as DNMTs (DNA methyltransferases), HATs (historic acetyltransferases), HDACs (historic deacetylases including siruins) (Bronner et al. 2007). If these systems are damaged and thus, rendered dysfunctional, the result can be erroneous epigenetic marks that have been shown to have roles in human disease such as cancer (Delcuve et al. 2009). ECM also is associated with a phenomenon referred to as gene bookmarking; this process is defined as “the process of remembering patterns of active gene expression during mitosis for transmission to daughter cells” (Sarge and Park-Sarge 2009). Chemical marks involved in gene bookmarking include histone post-translational modifications, DNA methylation at CpG islands, and small nuclear RNAs processes. Defects in ECM are postulated to reflect changes in gene bookmarking. Because this phenomenon only has been recognized in the past few years, much is unknown and remains to be discovered. It is believed that gene bookmarking is achieved through control of the degree of compaction of promotor regions of specific genes; for example, heat shock factor 2 protein (HSF2) binds promotor elements of heat shock genes, thereby bookmarking the region (Wilkerson et al. 2007, Murphy et al. 2008). Defects in one or more of these systems will lead to loss of function of ECM. The SR assay provides a model system for the investigation of gene bookmarking effects upon stress resolution pathways outcomes in human and animal cells.

it is believed that the SR assay is the first assay with the capacity to identify and quantify cells that have undergone permanent changes in ECM due to exposure to an agent or condition. The SR assay is capable of detecting and quantifying ECM functionality that ranges from fully functional with maintenance of the parental phenotype and resistance to permanent damage induction, to dysfunctional with delayed restoration of parental cell phenotype, to loss of function with expression of an altered phenotype. Some of these latter cells have been found to express a tumorigenic phenotype. In the SR assay, the frequency of occurrence of surviving damage tol-irr clones, and determination of the fraction of the surviving cell population that is composed of cells with this phenotype are used as quantitative indicators of the proportion of cells that have lost ECM function.

In summary, recent advancements in disparate fields of basic science research have shown that stress resolution processes, including but not limited to mutagenesis, are a complex processes. It is believed that mutagenesis is linked to stress response resolution processes mediated by several cellular pathways. The SR assay represents a significant departure from previously developed in vitro cell survival and mutation assays. While it does detect imitations, it also detects and quantifies the expression and/or outcomes of stress response resolution pathways, thereby making it possible to quantify the effects of an exogenous exposure upon the functionality of large groups of genes, accessory factors and elements. It is believed that the assay is novel in its ability to quantify epigenetic and chromatin modifications that affect cell phenotype. Importantly, the assay measures the outcomes of cellular processes that are hypothesized to have a direct relationship to the occurrence of several human diseases, including but not limited to cancer. The use of the SR assay will improve the current understanding of mechanisms leading to disease in humans, toxicology and risk assessment, and will enhance drug discovery, development, testing, and safety assessments.

Human Genotoxic Hazard Testing

Currently, testing of the outcomes of exogenous exposures is limited to agent/drug testing in vivo in animal models; such testing is limited by the high cost, long duration of the studies, and uncertainty in the results because of inherent differences in organs, sexes, strains, and species of animal models and between animals and humans, in addition, in vivo animal model testing provides only limited mechanistic information. There is a clear need for rodent and human models in which both targeted and untargeted mutagenesis can be studied, and in which the induction, progression, and stress response-related outcomes can be determined and quantified.

It generally is agreed that human genotoxic hazard testing is a necessary step in the development of pharmaceutical agents and is required to ensure their safety. Currently the testing battery agreed upon and used by the US, the European Union, and Japan is made up of a series of in vitro and in vivo genotoxicity assays; however, it is recognized that this battery of assays is limited in the ability to predict carcinogenicity (Simmons et al., 2009). Prokaryotic mutation detection assays, which are one of the standard tests currently used (e.g., the salmonella reversion assay), may be the easiest to perform, but the biology of these systems (including metabolic activation and gene expression responses) differ greatly from those of humans and other mammals, thus, introducing uncertainty into data extrapolation processes. Mutation assays of eukaryotic cells rely primarily upon animal models. These mutation assays using eukaryotic cells all share the limitation of having great difficulty evaluating mutation induction in epithelial or epithelial-like cells. This limitation is especially detrimental to the advancement of knowledge of mutagenesis and carcinogenesis because the majority of human cancers (lung cancer, breast cancer, prostate cancer, colon cancer, skin cancer, liver cancer), occur in cells of this type. This limitation also prevents testing and discovery of agents and/or drugs that protect against or inhibit mutagenic mechanisms. Currently available assays also are limited by lack of high throughput methodology and applicability to both animals and humans. In addition, none of these assays has the capacity to assess directly the effects of exogenous exposures upon the epigenome including effects upon chromatin conformation and ECM.

In June of 2007, a report of the National Research Council's Committee on Toxicity Testing and Assessment described their vision of the future direction of toxicity testing in the 21st-century (National Research Council (NRC), 2007, Toxicity Testing in the 21^{st} Century: A Vision and Strategy. Committee on Toxicity Testing and Assessment of Environmental Agents. The National Academies Press, Washington, D.C., 196 pp). The committee expressed its opinion that toxicity testing was poised to undergo major changes as a result of recent advances in the fields of toxicogenomics, bioinformatics, systems biology, epigenetics and others. They projected that Whole animal testing would be replaced to a large extent by in vitro methods that evaluated changes in biological processes using cells, cell lines, or cell components preferably of human origin. The report summary noted that the above scientific disciplines have advanced sufficiently to be able to identify cellular response networks consisting of intricate biochemical interactions between genes, proteins, and small molecules that maintain homeostasis. The term toxicity pathways was used to refer to cellular response networks that “when sufficiently perturbed are expected to result in adverse health effects”. The report authors envisioned novel toxicity-testing systems with the capacity to detect and measure key toxicity pathways of human cells and organ systems. Their new approach would address several current problems associated with toxicity testing including (1) the inability of most (all) currently available in vitro toxicity tests to determine the effect of the detected change(s) upon cell or organ behavior, (2) the high cost and resource-intensive nature of animal testing that limits the number of agents that can be evaluated and, (3) the uncertainty associated with extrapolation of animal data to humans. The SR assay is an in vitro cell culture assay with the above described characteristics that detects and quantifies the expression and functionality of stress response resolution pathways as examples of toxicity pathways. Thus, the SR assay can be used to meet the goals described above.

HPRT Studies

Of the five reporter gene assays for quantification of mutational burden in the DNA of human and animal cells (primarily T-cells), only HPRT has been extensively characterized, resulting in meaningful databases. Current understanding regarding the molecular bases of in vivo somatic mutations in humans and intact animals has been derived from this system. Popular acceptance of the HPRT assay stems from its quantitative read-out and the ability to obtain viable mutant cells for molecular and other analyses. The use of cytotoxic purine analogues to select for cells with HPRT mutations facilitates the isolation and molecular characterization of mutational events in T-cells. Many laboratories worldwide use this system. Background mutant frequency values have been quantified for all age groups, for the effects of smoking, and for other life style factors. Development of a computerized database of mutational spectra now is available to all workers in the field, based upon molecular studies performed on thousands of mutant isolates. Knowledge gained of T-cell activation, differentiation, and cellular function has allowed detailed mechanistic studies on unique populations of mutant lymphocytes, particularly in the clinical settings of autoimmunity, transplantation, and cancer. Adaptation and use of the HPRT assay for a wide variety of cell types, including epithelial cells, neurons, glial cells or any type, cardiomyocytes, endothelium, and stem cells of any type has not been achieved. The SR assay is the first quantitative assay that has been optimized to detect and measure the mutational burden of epithelial cell populations from humans or animals. These considerations also are applicable to detection of mutations in other reporter genes or genes of interest using the SR assay.

Three recent studies have found that repression or blockage of REV3 (the catalytic subunit of DNA polymerase zeta) expression resulted in a reduction of most or all of spontaneous and/or induced mutagenesis in the HPRT gene (Diaz et al, 2003, Li et al. 2002, Zhu et al. 2003). Since REV3 is a critical component of the TLS subpathway of post-replication repair, these findings suggest that mutation induction at the HPRT locus may be informative as a biomarker for expression of TLS. Because of the association between TLS expression and expression of specific stress resolution pathways, mutagenesis at the locus also may be informative as a biomarker for a switch in cellular expression from damage avoidance paths to damage tolerance stress response resolution paths, including both damage tol-rev and damage tol-irr paths and subpaths. The use of mutation induction in the HPRT gene as a biomarker for the above processes in any cell type is incorporated herein.

PIG-A Studies

The PIG-A gene is one of seven genes that code for the proteins constituting glucosamine acetyl (GlcNAc) transferase, a multimeric enzyme that mediates the first step in glycosylphosphatidylinositol (GPI) anchor biosynthesis. This GPI anchor is necessary for fixing a series of proteins (antigens) to cell surfaces; some of these proteins serve as complement regulators. Without the GPI anchor, none of the proteins is attached to the cell surface but, rather, the precursor is either degraded intracellularly or secreted extracellularly. The seven genes involved in this biosynthetic first step are PIG-A, PIG-H, PIG-C, GPI1, PIG-P, DPM2, and PIG-L. Only PIG-A, being located on the X-chromosome (at Xp22.1), is hemizygous (males having only a single X-chromosome, females having only a single functional X-chromosome); the other six are located on autosomal genes. In total, 11 reactions are required for GPI-anchor-protein fixation on cell surfaces. Most or all of the proteins involved in the pathway are required for intact GPI anchor production and loss of any of these proteins results in complete disruption. Thus, most or all pathway genes must be functional and the occurrence of GPI-linked proteins on cell surfaces, while an easily detected phenotype, has a complex genetic basis.

The PIG-A gene has a genomic length of 17 Kb, making it an average size gene. The mRNA is 1600 bp, with an open reading frame of 1455 bp. The gene consists of 6 exons: exon 1 of 23 bp constitutes the 5′ untranslated region; exon 2 of 777 bp makes up approximately one half of the coding region; exons 3, 4 and 5, which are coding regions of 133, 133 and 207 bp respectively, and exon 6, which constitutes both a coding and the 3′ untranslated region of 2315 bp. In addition, the 583 bp 5′ flanking region has promoter activity. The predicted protein is 484 amino acids starting at bp86, GPI anchored proteins have clinical significance in the human somatic cell genetic disorder paroxysmal nocturnal hemoglobinuria (PNH). In PNH, one or more clones of RBCs arise that are deficient in GPI anchored proteins, enhancing their susceptibility to complement-mediated lysis. The origin of the clone or clones is somatic mutation of the RIGA gene. An inactivating mutation of any of the genes in the GPI anchor biosynthetic pathway potentially could have produced the same result, but only mutations of the PIG-A gene have been found in the hundreds of cases examined. The X-chromosomal location of PIG-A ensures that one inactivating mutation in the gene will result in the PNH phenotype.

PIG-A mutations in PNH have been characterized at the DNA sequence level. Most have been “point mutations”, including base substitutions and small deletions and insertions. A large number of these have been frameshift mutations. Some large deletions/insertions have been seen. All exons, except exon1 have been affected.

Mutagenesis in the PIG-A gene can be evaluated by testing for the presence or absence of GPI-anchored proteins, or by testing for the presence or absence of antibodies that bind the GPI anchor itself. Antibodies for specific GPI-anchored proteins are used to distinguish between the presence and absence of the specific protein on the cell surface; flow cytometry is used to quantify numbers of cells in each category. In all cases, antibodies with specificity for the proteins to be bound of the species from which the cells were derived must be use; for example, anti-rat CD59 might or might not have affinity for human CD59. In cells of hematopoietic origin, CD59 (for RBCs) and CD48 (for lymphocytes) have been used as reporters of PIG-A mutations. The fluorescent reagent that specifically binds GPI anchors (FLAER) has been used to detect the frequency of GPI-deficient RBCs. Previous work by other investigators has shown that genotoxins increase the frequency of GPI-anchored-protein-deficient cells and that these cells have mutations in PIG-A (Miura et al. 2008a,b). Methods for detection of GPI-anchors or GPI-anchored proteins have been published previously, but methods for the detection of these proteins in epithelial cells have not been developed or published, and none of these techniques have been used in conjunction with the SR assay to achieve the goals or applications of that assay.

Detection of PIG-A mutations using flow cytometric methods has the advantage that it is not necessary to expose the cells to a stress response induction/selection agent. Such exposures are used to identify and quantify cells with mutations in a population of wild-type and mutant cells. However, the high levels of cytotoxicity, which are needed to remove all or most of the cells lacking mutations, induces a cytotoxic stress response, and probably other stress responses as well, thereby confounding the results where determination of mutation frequencies in the absence of stress response-associated mutagenic processes is the goal. Thus, determination of PIG-A mutant frequencies by flow cytometry in the SR assay can be performed before stress response induction, and again after stress response resolution has been completed, for the purpose of determining the impact of the stress resolution pathways upon the mutational burden of the cell population. Additional genes probably will be identified in the future that can be used as reporter genes for assessment of mutagenesis. Methods for evaluation of mutagenesis in these genes may be developed that will allow the use of these genes as endpoints for assessment of mutagenesis in the SR assay. The use of new or novel gene targets for this purpose is incorporated herein.

To achieve the goal of using PIGA mutant frequencies as indicators of mutational burden in a cell population before and after stress response induction, as is done in the SR assay, it is necessary to recognize that several cell types display the phenomenon referred to as auto-fluorescence when exposed to one or more of the wavelengths of light used for flow cytometric determination of cell population subgroups. This problem often is reported for epithelial cells from differing tissues and also is associated with changes in gene expression, development of some disease conditions, other cellular phenomena and varies between cells of the same type from different individuals. In addition, autofluorescence may be observed at one or more wavelengths. A battery of methods has been developed to address this problem. These correction methods fall into several categories including subtraction of estimated autofluorescence at wavelengths above 580 nm, ratiometric methods that allow separation of autofluorescence from antibody or stain fluorescence, use of excitation wavelengths at which autofluorescence is reduced or eliminated, use of multiple fluorescent tags along with one or more of the above methods, and others. In addition, new methods for addressing problems associated with flow cytometric analysis of cell populations, including methods to address autofluorescence-associated problems, are being developed and/or refined frequently, so new methods for addressing these issues are expected to be described and developed in the future. These existing and to-be-developed methods for identification and quantification of PGA mutations, with resolution of autofluorescence problems in human and animal cells, are incorporated herein.

Another problem that must be overcome stems from the fact that some genes involved in GPI-anchor biosynthesis can be silenced by epigenetic mechanisms and this silencing can lead to failure of the cells to express the GPI anchor or to express GPI-bound proteins on the cell membrane. To circumvent this problem, re-expression of the epigenetic silenced genes can be induced by one of several methods that have been described in the literature, including but not limited to the use of de methylating agents. Additional methods and techniques for inducing expression of epigenetically silenced genes are expected to be developed in the future. The use of these methods for the purpose of inducing expression of epigenetically silenced genes for use with flow cytometric evaluation of cells for evaluation of mutation and epigenetically alterations in GPI-anchor biosynthesis genes is incorporated herein.

It has been found that improved sensitivity of identification of PIG-A mutation frequencies is obtained if analyses are carried out that assess the presence or absence of the GPI-anchor itself and also the presence or absence of GPI-anchored proteins. It is important to note that the profile of GPI-anchored proteins varies between cell types and between species. Since the SR assay is applicable to a wide range of cell types from different species, it will be necessary to identify GPI-anchored proteins specific for the cell type being studied in the assay, so that fluorescent stains specific for these proteins can be used for determination of PIG-A mutant frequencies. These techniques are incorporated herein.

The mutational spectrum of PIG-A mutant clones reveals mutation types that are characteristic of expression of stationary phase mutagenesis, a stress-associated mutagenic process. These observations support the postulate that mutation induction in the PIG-A gene may be informative for expression of stationary phase mutagenesis and/or other environmental stress-associated mutagenic processes. Recent studies have found that repression or blockage of REV3 expression resulted in a reduction of most or all of spontaneous and/or induced mutagenesis in the HPRT gene (see above) it is possible that spontaneous and induced mutagenesis at the PIG-A locus also is informative as a biomarker for expression of the TLS subpathway of post-replication repair and also as a biomarker for a switch in cellular expression from damage avoidance paths to damage tolerance stress response resolution paths, including both damage tol-rev and damage tol-irr paths. In other words, mutation induction at the PIG-A locus may be informative as a biomarker for the same processes as described above for the HPRT gene. The use of mutation induction in the PIG-A gene or other gene loci, which can be assessed using methods that avoid induction of a stress environment as part of the protocol, as biomarkers for the above processes in any cell type is incorporated herein.

General Considerations

Poisson Distribution Principles, Hypotheses, and Limitations:

The SR assay uses the same assumptions, hypotheses and limitations that are described for the T-cell (HPRT) cloning assay, the Tk assay, the PIG-A assay and other gene locus-specific assays. Abiding by these constraints makes it possible to utilize the mathematic formulas developed for quantification of cloning efficiency(ies) (CEs) and mutant cell frequencies as is done in the T-cell cloning assay (Haynes 1989). The SR assay design and methods must conform to Poisson distribution principles and limitations, so that Poisson distribution-based statistical and mathematical methods can be applied. These principles include (1) the cells to be evaluated in the assay must be rendered into a single cell suspension, (2) the cells must be evenly distributed across the cell suspension, (3) the raw data derived by application of the assay must be binomial in nature, and (4) the raw data cannot be of only one type (in other words, the binomial data must consist of both positive and negative data points). These assumptions, hypotheses, principles, theories, conditions and limitations, as applied in the SR assay, are incorporated herein.

Applicable Cell Types

The SR assay is applicable to all mammalian cell types, either primary or immortalized, with the following characteristics: (i) the cells can be cultured in vitro for a period of time sufficient to complete the assay (cell immortalization may be required to meet this criterion), (ii) the cells are sensitive to the stress induction effects of the calibrated stress conditions (see above for definition), (iii) the cells lack pre-existing damage in reporter genes of interest used in the assay as indicators of the persistent damage burden in DNA (currently these genes are the HPRT gene and the PIG-A gene) (this condition only needs to be met for cases where assessment of mutations in the reporter gene(s) of interest forms part to the goals of the study), (iv) the cells resolve the calibrated stress conditions using pathways with outcomes that can be identified and quantified, in the form of binomial data. To date, primary rat lung epithelial cells, immortalized murine mammary epithelial cells, immortalized murine alveolar type II lung cells and immortalized human bronchial epithelium have been used successfully in the assay. Cells to be used in the assay also should be evaluated to determine that they respond to the stress inducing agent with expression of cell cycle arrest, cell death, or survival or any other endpoints that are planned for use in the assay.

Human and animal cell types of interest for which quantitative assays of stress response resolution pathways have not been available previously include, but are not limited to, human and other mammalian epithelial cells from any organ or tissue, glial cells of any type, neurons of any type, cardiomyocytes, cells of the immune system, including but not limited to dendritic cells, stromal cells of any type, fibroblasts and fibrocytes, stem cells including but not limited to embryonic stem cells, and endothelial cells from any organ or tissue. Currently little is known about stress response induction and resolution in these cell types and the application of the SR assay to evaluate stress response resolution pathways in these cells types and under a variety of conditions of importance to human health will provide novel information. This application of the SR assay is incorporated herein.

While the assay can be applied to primary, non-immortalized cells, the use of such cells can make it difficult to detect some cell clones that survive the calibrated stress conditions step of the assay by expression of damage tolerance pathways since these clones may be delayed in their appearance in the assay, and may undergo senescence prior to being detected. This difficulty is an in vitro cell culture phenomenon reflecting reduced cell survival during in vitro culture, and does not reflect in vivo cell survival. Because of these considerations, it is advisable to immortalize primary cells using any available cell immortalization technique, including but not limited to infection with viruses (such as the human papilloma virus or SV40) or through transfection with plasmids carrying transcripts that induce expression of an immortalized phenotype prior to the start of the assay such as the hTERT plasmid for human cells. Primary cells can be exposed and then immortalized; cells also can be exposed in vivo in an animal model and then can be harvested and immortalized prior to beginning the assay. Cells with or without a functional p53 gene can be used successfully. Other immortalization techniques also may be used, including techniques that are discovered or recognized in the future; these techniques are incorporated herein.

The use of existing, rapid, high throughput methods for identification of clones expressing damage avoidance versus damage tolerance pro-survival pathways will be evaluated for use with the SR assay in the future; the application of these methods to the assay is anticipated to reduce or eliminate the need to immortalize cells for use in the assay. The use of these methods in conjunction with other components of the assay is incorporated herein.

In Vitro and In Vivo Applications

Cells cultured in vitro for the entire course of the assay can be used. Cells taken from an animal or human also can be used. The initial or pre-exposure step of the assay, if planned as part of the assay, can be carried out in the animal model or can have occurred in the human being prior to the collection of cells for use in the SR assay.

Types of Cellular Stress Response Inducing Agents/Conditions that can be Evaluated in The SR Assay; Types of Cellular Stress Responses that can be Evaluated

In general, any agent or condition that alters cell function and/or expression of stress response and resolution outcomes in ways that permit identification of and separation of cells with differing stress resolution responses and that meets the requirement that the outcome data be binomial in nature can be used. In general, any cellular stress response that meets the above requirements can be investigated using the SR assay (see Table 4). One of several ways of meeting the requirement for binomial data is to use agents that induce senescence or cell death, in a large portion of the cells, with the remainder of the cells expressing a survival response. Then other changes in surviving cells can be evaluated for the purposes of further sub classifying the surviving cells. However, any other selection criteria that meet the above basic requirements can be used. As an example, agents that alter cell metabolism-related or cell transport-related stress resolution pathways also could be used. As another example, agents that have been recognized to provide a survival advantage to cells with mutations or alterations in reporter genes other than HPRT or PIG-A can be used to identify SR assay surviving clones with alterations in these genes. Glucose deprivation and cell crowding have been used successfully as SR assay-induced stress conditions. The calibrated stress conditions agent that has been used most frequently during development of the SR assay is 6-thioguanine (6-TG), a nucleoside analog and analog of guanine that is incorporated into DNA, thereby inducing a DNA damage response. This agent also probably induces a cytotoxic stress response, a mitochondrial stress response, and a nucleotide pool imbalance stress response; induction of other stress responses also is possible. Other DNA damaging agents and other stress response-inducing agents can be used in the assay.

Because of concerns about the effects of agents that damage or alter the genome or the epigenome, thereby leading to effects upon gene expression, cell function and disease occurrence, it is anticipated that the SR assay will be used frequently to evaluate the effects of agents or conditions that are known or suspected to alter or affect the expression and functionality of DNA damage response-associated resolution pathways. It is important to note that current knowledge supports the postulate that a variety of stress environments, inducing agents and conditions that do not induce a DNA damage response, are resolved using some or all of the pathways described above (see background; stress resolution pathways and outcomes) (reviewed in Simmons et al. 2009). As an example, both glucose deprivation and cell crowding are resolved, at least in part, through the expression of damage tolerance stress resolution pathways. Table 4 has a list of stress environments and/or stress response-inducing agents or conditions that can be used during the calibrated stress conditions phase of the assay and for which there is evidence that resolution uses some or all of the components of pathways used by cells to resolve a DNA damage response. More than one type of stress response can be induced simultaneously to achieve the SR assay goal of evaluating the outcome of two or more types of stress environments simultaneously in cells of interest.

Since new information is being discovered continuously about agents or conditions that lead to stress in human and animal cells, and about the cellular processes involved in resolving these stress conditions or challenges, other types of stress, other stress-inducing agents or conditions, and other resolution pathways will or may be discovered in the fixture that can be evaluated in the SR assay. In addition, other inducing agents or conditions and responses to those agents or conditions may be described in the future with outcomes that can be assessed using the SR assay. These stress-inducing agents or conditions, or types of stress responses and their resolution pathways are incorporated herein.

Determination of Calibrated Stress Conditions and SRID-X % Level

The calibrated stress conditions and the SRID-x % level are determined ideally using parental, control, or vehicle-exposed cells. This portion of the assay requires that several variables, including cell plating density, calibrated stress conditions level, and duration of stress conditions exposure be optimized simultaneously. These variables are interdependent such that each affects the outcome of the other, thereby making this portion of the assay one of the most critical. To achieve high sensitivity for detection of assay endpoints, it is important that the maximum number of cells be evaluated; however, optimal calibrated stress conditions are achieved at relatively low cell plating densities. In addition, cell plating densities and the SKID-x % level of stress inducing agent chosen for use in the assay must be such that fewer than one positive endpoint per well occurs in the assay. Using cell survival as an example, if multiple surviving cell clones are identified in the same well(s) during an experiment, then the cell plating density must be reduced and/or the SRID-x % level must be increased so that the frequency of occurrence of surviving clones is reduced to less than one per well. Thus, optimization of these variables is necessary.

In general, the calibrated stress conditions should meet the following criteria: (1) the conditions should be high enough to induce a stress response in target cells, (2) the conditions should be as low as possible so as to reduce damage induced in cells as a result of the stress conditions exposure (this may vary with the goals of individual experiments), (3) if alterations in reporter genes are included as part of the SR assay, the conditions should be such that recovery of cell clones with alterations in the reporter gene is optimized, (4) the conditions should conform to the criteria for setting the SRID-x % level for any experiments so that the design conforms with Poisson distribution principles (see above).

Selection of the optimal calibrated stress conditions level will depend upon the goals of the particular study. Table 2 summarizes the four major levels of stress response induction that have been recognized in a range of eukaryotic cell types and that can be induced as part of the SR assay. The calibrated stress conditions level is an important component of the assay because it affects the types of outcomes that will be expressed by the cells under study.

Pilot studies that evaluate a range of cell plating densities and a range of calibrated stress conditions levels for the stress inducing agent of choice are used and are applied to untreated, control or parental cells. It is advisable to use a low plating density that results in rapid expression of stress resolution pathways (within 3 to 4 days where possible), or as quickly as possible after calibrated stress conditions exposure. In spite of potentially significant differences between cells of different types from different tissues and from different species, preliminary results from the SR assay support the conclusion that the optimal plating density for many cell types falls within a predictable range. Epithelial cells that have been tested in the assay showed a visible SR induction response within 48 to 72 hours after the start of the calibrated stress conditions phase when plated at 100,000 to 200,000 cells on 100 mm tissue culture dishes, or at 500 to 600 cells per well of a 96 well tissue culture plate, or at comparable densities on other tissue culture vessels. Lymphocytes are estimated to respond similarly when plated at a density of 20,000 to 40,000 cells per well of a 96 well tissue culture plate, or at comparable densities on other tissue culture vessels. Lymphocytes and some other cell types do not attach to the tissue culture vessel and thus, they must be evaluated in wells or other similar cell culture system. These values can serve as starting points for empirical determination of the optimal cell plating density for the cells of interest to be used in the assay.

After estimating the optimal cell plating density, dose response curves are generated for the calibrated stress conditions agent of interest. For experiments in which cell survival is the phenotype of interest, work with the assay completed to date supports the conclusion that calibrated stress conditions levels that reduce expression of pro-survival pathways to a level that results in survival of one or fewer clones in 50% of the wells of a 96 well plate (defined as the SRID-50%) is the optimal level for assessment of damage avoidance pro-survival pathways. Use of these levels also results in a sufficiently robust expression of damage tolerance paths to make it possible to evaluate differences or changes in expression of these pathways between cell types and/or between differing exogenous exposures. Because cell plating density and level of calibrated stress inducing agent are inter-dependent, it may be necessary to perform several pilot studies using differing cell plating densities and a range of stress-response inducing levels to find the optimal combination of use with the cell type of interest in the assay.

There are two limits associated with Poisson distribution principles that must be considered in selecting the SRID-x % level of stress inducing agent. First, stress induction levels that result in a response frequency, for the response type of interest, that is near or equal to 100% of the sites being evaluated should be avoided and the same considerations are true for stress induction levels resulting in responses frequencies near or equal to 0%. The SR assay cannot accurately quantify response frequencies near 100% or near 0%, and this limitation forms the one of the factors affecting the selection of the SRID-x % level. Due to Poisson distribution considerations, the optimal value for x (defined as the ratio of negative wells to total wells of a tissue culture vessel and expressed as a percentage) is 50%. The calibrated stress conditions that meet this criterion are termed the SRID-50%. Since this value is affected by duration of exposure to the calibrated stress conditions and cell plating density, information about these conditions should be included when describing SRID-x % levels used in the SR assay.

Certain types of stress-inducing agents and/or conditions such as nutrient deprivation, cell crowding, or physical pressure initiate a cellular stress response over a much longer period than that observed with other types of agents such as chemicals or radiation. For these former agents, determination of the optimal level for induction of a stress environment, such as determination of the SRID-x %, may be difficult. To address this issue, it may be necessary to perform the SR assay using a range of stress environment levels to identify the optimal level at which the cells of interest resolve the stress environment using predominantly damage avoidance pathways or a range of resolution pathways.

Additional considerations involved in the choice of calibrated stress conditions levels include the following. In order to evaluate the expression and functionality of multiple stress resolution pathways and their outcomes, including the effects of an exogenous exposure, it is advisable to use a calibrated stress conditions level that corresponds to the moderately low/acute level effects listed in Table 2. Use of this level optimizes damage avoidance paths expression and enhances the detection of effects resulting in the expression of damage tolerance stress resolution paths that may occur as a result of an exogenous exposure. In addition, it was found that HPRT⁻ clones were detected in the assay at optimal levels when calibrated stress conditions levels were in the range of SRID-10% to 50%. The use of significantly higher calibrated stress conditions levels, such as those in the range of SRID-70% to 80%, resulted in significant reduction or complete loss of the ability to detect HPRT⁻ cell clones. Finally, use of high level and/or prolonged calibrated stress conditions levels may preclude expression of damage avoidance paths while enhancing expression of damage tol-rev and damage tol-irr paths, thereby, precluding the evaluation of these damage avoidance pathways in the SR assay. Some gene expression pathways that have been shown to operate at these two levels are mutually exclusive, such as up-regulation versus down-regulation of DNA repair pathways, supporting the conclusion that some components of damage avoidance and damage tolerance paths cannot be expressed simultaneously.

In most of the examples given below, an empirically-determined short-term, moderate intensity stress environment near or equivalent to the SRID-50% or the SRID-10% for vehicle-exposed cells was induced because this optimized expression of damage avoidance pathways and total cell survival of control cells, which was the response type of interest in the assay, while keeping total cell survival within the range of the limits described above. Conditions that optimized cell survival were found to be superior for investigation of the three major groups of pro-survival stress resolution pathways described above, including but not limited to the recovery of HPRT⁻ surviving clones. Use of higher stress response induction levels resulted in the survival of predominantly irreversible phenotypically-altered clones and reduction or loss of expression of other types of surviving clones, thereby limiting the information that could be derived from the SR assay. The use of calibrated stress conditions that optimize the recovery of the maximum number of cells that display the phenotype of interest is advisable because these conditions lead to consistent, reproducible results between experiment replicates and also provide the most amount of information about pathway expression and functionality.

The calibrated stress conditions should be sufficiently high to induce expression of stress responses in the cell type of interest. Calibrated stress conditions that do not induce stress response expression in all or a large majority of the cells may not be useful in the SR assay.

The use of molecular markers of stress induction is advisable for demonstrating that a stress response was induced in the cell type of interest, and for characterizing the nature at the molecular level of the induced response, and/or for calibrating the intensity and nature of that stress response. Molecules currently recognized to have a role in stress response induction and/or early stages of SR resolution include ATM (ataxia-telangiectasia mutated), AIR (ATM and Rad3-related), Chk1 and Chk2, and p53 and DNA damage response-inducible proteins, including but not limited to TNF-alpha, p16^{INK4A} and p21^{CIP1A} (see Simmons et al, 2009 for additional markers). Because of research in these areas, it is probable that additional molecular markers of stress response induction will be identified and/or improved methods of detection and quantification of the markers will be developed. Any methods currently available for determination of the expression levels of these molecules may be used, including any methods that may be developed in the future. In addition, levels of other molecules with roles in stress response induction, progression, or resolution also may be identified in the future and may be used as part of the SR assay to monitor and/or assess the nature of the inducted response. Testing for the expression of stress response-related molecular biomarkers or for a role of known and/or to-be-identified molecules with roles in stress response induction, progression, and/or resolution is incorporated herein when used in the context of performing the SR assay and/or one or more of its subcomponents.

SR Assay Raw Data, Calculated Data, and SR Assay-Derived Materials

Raw data derived using the SR assay must be binomial in nature. Currently this condition is met by assessing cell survival versus cell death at discrete sites on tissue culture dishes or in wells on tissue culture plates. This raw data is binomial because the sites where cells are plated for evaluation are scored as either positive or negative. Thus, the SR assay provides raw data upon which calculations are based to quantify assay outcomes, but the assay also provides assay-derived materials in the form of viable, senescent, or dead cell clones that can be subjected to further evaluations. Thus, SR assay results come from several sources, including (1) the raw data and the calculations based upon the raw data, (2) assessments based upon the calculated results, and (3) additional assessments based upon evaluations of surviving and/or non-surviving cells.

The SR assay derived binomial data and materials can be subjected to the evaluation of additional endpoints. These endpoints can include, but are not limited to (1) the types and levels of DNA damage at specific gene loci, (2) the types and levels of epigenetic alterations, (3) types and levels of alterations that result in phenotypic and/or functional alterations in cells, (4) stress response pathway expression and functionality (see below) both before and after SR induction and resolution, and (5) cell population composition changes that occur in surviving cell populations after stress response resolution. In addition, any other endpoints of interested can be assessed using SR assay-derived results or materials. The overall purpose of these endpoints is to assess and/or to quantify further the expression of stress resolution pathways, to measure their functionality, and to quantify their outcomes. Molecular indicators or biomarkers of endpoints of interest can be used to enhance the information obtained. In addition, the assay design makes it possible to determine if pre-exposure to a test agent or condition, followed by SR induction, alters the expression and/or functionality of stress response resolution pathways with resultant changes in the final outcome, and allows for the quantification of induced changes. For this purpose, it is valuable to evaluate endpoints both before and after the induction of the calibrated stress conditions of the assay. The use of SR-assay derived results (calculated results and/or assay-derived materials) for performing additional endpoint assessments are incorporated herein.

Specific Methods for Performing the Assay

The SR assay consists of three major phases, with several steps involved in each phase. The phases are referred to here as (1) the pre-calibrated stress conditions exposure phase, and this phase may contain an initial exposure step and midway assessment steps, (2) the calibrated stress conditions exposure phase, and (3) post-calibrated stress conditions exposure and final assessments phase. The steps associated with each phase are described below. A summary of the phases of the assay, their associated steps, and the endpoints evaluated is presented in Table 7. The assay can be performed in whole or in part; in other words, the assay can be used to evaluate cells for changes in only one or more of the endpoints described below.

Phase 1: Pre-calibrated stress conditions exposure, including initial exposure step(s), adaptive response period, and midway assessment phase:

Phase 1 step 1: Evaluation of the cells to be used in the assay.

Cells to be used in the assay should be evaluated as described above to be sure that they meet the criteria for successful use in the assay. Conditions for optimal growth must be determined and used during the performance of the assay.

If HPRT mutant clones are to be evaluated as part of the assay, the parental cells must be tested to determine the optimal concentration of aminopterin to use as the cytotoxic component of HAT-medium, if phenotypic testing of Phen-HPRT⁻ clones is planned. Cellular responses to aminopterin vary by cell type and may be dependent upon expression levels of the c-myc gene, so each cell type must be tested so that the optimal concentration of HAT-medium can be used below. If necessary, cell immortalization is carried out during this portion of the assay.

Phase 1 step 2: Determination of the SRID-x-% level for the calibrated stress conditions agent selected for use in the assay; (establish cell plating densities and exposure conditions necessary to achieve desired calibrated stress conditions level).

Table 1 shows the types of stress response resolution pathways expected to be expressed by cells following exposure to differing levels of calibrated stress conditions. The amount of calibrated stress conditions agent necessary to induce expression of one or more resolution pathways must be determined empirically and is expected to vary depending upon cell type and other factors. The methods for achieving this are described above. SRID-x % levels are determined using parental, control, or vehicle-exposed cells.

The majority of toxicity testing reported in the literature appears to have been carried out using exposure levels that fall into the high-level, prolonged, and/or chronic calibrated stress conditions category, albeit without recognition that these exposures were eliciting stress responses in the test cell population(s). Use of these levels for stress response induction may preclude the ability to observe damage tolerance pathway expression, and thus, the impact of exogenous exposures upon the expression and functionality of these paths cannot be determined with confidence at these levels of calibrated stress conditions.

Phase 1 step 3: Initial exposure (or pre-exposure) step (if part of the planned experimental protocol) and adaptive response/resolution period (phenotypic expression period)

One of the applications of the SR assay includes its use to determine the effect(s) of an exogenous exposure upon the expression and functionality of stress-resolution pathways. The calibrated stress conditions phase of the assay can be performed after a cell population, which either is cultured in vitro or is present in a living human or animal, has been exposed to an agent or condition of interest. In addition, cells can be exposed during all or part of the performance of the assay to determine the effect of such exposure conditions upon SR assay results. This “initial exposure” phase of the assay may result in induction of a stress response in the exposed cell population; this stress response is not considered to substitute for the calibrated stress conditions exposure phase of the assay.

When the SR assay is used to evaluate the effects of pre-exposure to an exogenous agent or condition, a time period between the end of the exposures and the initiation of the assay should be allowed to elapse. This is referred to as the adaptive response/resolution period or the phenotypic expression period. The duration of this period cannot be defined definitively for all cells types, but it is considered generally to be a period of time that corresponds to at least two cell divisions. The purpose of this time period is to allow for changes in cell protein levels or profiles to occur so that they reflect changes induced by the exogenous exposure. In some of the examples given below, the SR assay was performed using vehicle- and ethylnitrosourea- (ENU-) exposed cells. The cells that received the initial ENU exposure displayed transient changes in cell morphology and metabolism indicative of an induced stress response; the cells were expanded in growth medium without additional manipulations until these changes appeared to have stabilized, and then the SR assay was performed. The duration of the adaptive response period of the assay can be varied according to the wishes of the assay operator, or can be eliminated entirely if desired. This will, however, affect SR assay outcomes.

Phase 1 Step 4: Midway Assessments

Midway assessments can be made for any published or reported endpoint of interest, using methods appropriate for the selected endpoint(s). Note that the midway assessment endpoints are not limited to binomial data (with the exception of data used to calculate mCEs) The testing described here can be performed before, after, during, and/or before and after exposure to an exogenous agent is completed, according to the goals of the particular experiment and/or investigators. The midway assessments at this step of the assay serve the purpose of determining endpoints for comparison to endpoints assessed during the final assessment step of the assay and for determining the effects of the initial exposure (if performed); thus, if desired, the only midway assessment that needs to be performed is the determination of the in CEs

Midway assessment endpoints that have been evaluated include (1) DNA damage types and levels, (2) epigenetic modification types and levels, and (3) stress response pathway expression and functionality, (4) pro-survival pathway function in the form of CEs. For measurement of DNA damage, assessment of gene-specific damage has been used. Any method of determining DNA damage that does not involve exposure of cells to a stress environment can be used PIG-A mutant frequencies determined by flow cytometry currently can be evaluated as part of the midway assessments. In addition, determination of mutation induction at specific gene loci, for example hot-spots in the p53 gene, also may be used to achieve the goal of measuring DNA damage. A qualitative assessment of the parental cell phenotype currently is used as an indicator of the baseline status of the epigenome, based upon the recognition that cell phenotype reflects the status of the epigenome including but not limited to chromatin. The criteria listed in Table 5 are used to evaluate clones for classification as Phen-parental clones. Currently the SR assay does not include a molecular analysis of pre-calibrated stress conditions exposure epigenetic characteristics, but inclusion of testing procedures designed to provide these characterizations and to identify informative biomarkers is planned for future work and is included herein. All of these gene expression endpoints also can be evaluated before and/or after pre-exposure to a test agent or condition when a pre-exposure is planned as part of the experimental design. Other endpoints of interest can include, but are not limited to, assessments of microRNA expression and function, and/or assessments of mitochondrial integrity and function. Determination of mutations at gene specific loci can be a valuable method for assessing DNA damage before induction of the calibrated stress conditions exposure step of the assay. However, any methods chosen to obtain this data must avoid the use of selection or stress response induction methods, because these methods will give information about post-stress response induction mutations, but not pre-stress response induction methods. Assessment of PIG-A mutant frequencies, as a measure of DNA damage levels, may be especially useful aas part of the midway assessment because a method that avoids a selection or stress induction step have been described (see below for specific methods). Determination of pre- and post-calibrated stress conditions PIG-A mutant frequencies, using methods such as flow cytometry, which do not involve the use of a SR induction/selection phase, will be performed as part of future applications of the SR assay and is incorporated herein. Determination of HPRT mutant frequencies before calibrated stress conditions exposure will be carried out if a method that does not use a SR induction/selection phase is developed in the future. Other DNA endpoints or biomarkers that are developed in the future for assessment of DNA damage also can be used if these endpoints can be assessed without induction of a stress response.

Epigenetic alterations currently are evaluated in a qualitative manner by assessing the characteristics of the vehicle- and exogenous agent-exposed cell populations (if both are being used in the assay) for differences or similarities in the criteria listed in Table 5. Currently, the midway assessment epigenetic alterations are only qualitative, but future work will identify biomarkers that can be used to make this component of the SR assay quantitative. The use of such biomarkers is incorporated herein. Evaluation of molecular markers of cell function also can be used in this phase of the assay and their use is recommended. Their use is incorporated herein.

Midway pro-survival stress response pathway function is assessed by determining mCEs for the cells of interest including both vehicle- and test agent-exposed cells using one of the methods provided below. Determination of mCEs is used as a quantitative indicator of the expression and functionality of pro-survival pathways in the cell population(s) of interest under non-stress environment conditions. GE measurements allow comparisons between cell populations that have received different treatments or exposures, such as vehicle-exposed versus test agent-exposed groups of cells. mCE assessments are a required component of the assay since their calculated values are used in equations for calculating the fCE values at the end of the assay. In addition, SRID-x % values can be determined for each cell population being evaluated in the assay if desired using the stress-inducing agent selected for use in the assay.

In the future molecular endpoints or markers may be discovered or developed that can be used in lieu of determining CEs, and/or SRID-x % levels. The use of such endpoints may enhance the speed and accuracy of the assay but their use remains consistent with the general goals of the assay and their use as part of the SR assay is incorporated herein.

Phase 2: Calibrated Stress Conditions Exposure; Start of Stress Resolution

Phase 2 Step 1: Start of the Calibrated Stress Conditions Exposure.

In this step, cells are plated at the optimal density as determined in phase 1 of the assay and exposed to the calibrated stress conditions agent at the desired exposure level and duration as determined in phase 1. During this phase of the assay, all tissue culture vessels are evaluated visually and/or by microscopy to determine that changes indicative of early cellular responses to stress, such as cell cycle checkpoint arrest, have occurred. Cells should be maintained and re-fed during this period as necessary for the particular cell type or cell line being investigated in the assay. Exogenous agent exposures can be initiated or continued during this phase of the assay if desired as part of the specific experimental protocol.

Phase 2 Step 2: Cessation of the Calibrated Stress Conditions Exposure; Initiation of Resolution.

At the time point determined above for cessation of the calibrated stress conditions, the stress inducing agent is removed and the cells are fed with growth medium. Conditioned medium may be used at this point and is recommended for most or all cell types. This step may be difficult to perform for cells that grow in suspension and may require washing the cells, but the goals of this step should be met to the greatest extent possible. Subsequently, cells and/or cell clones displaying the phenotype of interest (such as survival) are identified and the number of surviving clones is counted. As part of this process, positive (viable, growing cells present) and negative (no viable, growing cells present) sites or wells are identified and scored as such. The exact timing of these identification and scoring steps will vary depending upon the cell type used, the type of test agent or condition being evaluated (if part of the assay), the nature of calibrated stress conditions agent, and other factors specific to each experiment. For experiments in which cell survival is the phenotype of interest, this phase of the assay can last for six or slightly more weeks. During this time period, the tissue culture vessels must be evaluated repeatedly for the purpose of identifying the appearance and expansion of surviving cell clones or cell clones with the phenotype of interest. It is advisable to record the time of appearance of these clones as this timing may be informative for the expression of specific stress response resolution pathways. For epithelial and epithelial-like cells, it also is advisable to record the morphology and/or growth rate of the clones as this correlates frequently with the sub-group testing that is performed in phase 3 of the assay. It is believed that longer latency periods from the cessation of the calibrated stress conditions exposure to re-entry into the cell cycle and expansion correspond to expression of damage tolerance as opposed to damage avoidance stress resolution pathways. During the time period in which this step of the assay is being completed, the cells should be maintained and re-fed as necessary for the cell type being investigated.

In the studies that have been performed to date, the timing of appearance of the clones correlated with their sub-group classification. Phen-HPRT⁻ clones appeared first and were recognizable as presumptive Phen-HPRT⁻ clones by their parental cell morphology and their slow growth. Phen-parental cell clones appeared around the same time and were recognized by their parental cell morphology and their robust growth rates. Phen-alt clones appeared last and were recognized by their frequently altered cell morphology and their robust growth. These guidelines may be informative for other studies; the use of other stress-inducing agents or conditions, and assessment of other endpoints is anticipated to introduce variations in the above described timing.

Phase 2 Step 3: Raw Data Collection, Identification of Viable Cell Clones

For SR assays in which survival is the phenotype of interest, cell clones should be removed from the tissue culture vessels when they have reached a size that is consistent with the presence of sufficient cells to survive re-plating, additional testing, and sub-classification. For other phenotypes of interest, the cells should be removed and/or subjected to further evaluations at a time that is optimal for initiation of testing and/or further stub-classification. This will depend upon the characteristics of the cells being evaluated, the nature of any exogenous exposures being evaluated (if any), and the nature of the calibrated stress conditions agent or condition. Removal of cells from the tissue culture plates and replating should be done using methods that are optimal for the cell type of interest and will vary by cell type. For epithelial cells, clones that had reached 30% or greater confluence were removed successfully and transferred to new tissue culture vessels for sub-classification testing.

Phase 3: Post-Calibrated Stress Conditions Exposure; Clone Sub-Classification by Phenotype, Final Calculations and Assessments

Phase 3 Step 1: Completion of Raw Data Collection; Recovery of Clones; Classification of surviving clones by phenotype.

Following the cessation of the calibrated stress conditions exposure, the SR assay raw data is collected in the form of positive and negative wells (or cell growth sites) and in the form of viable or dead cell clones. The next steps of the assay involve expanding viable clones and determining their phenotypes. When the phenotype of interest being investigated in the SR assay is cell survival, the clones can be sub-classified using the testing listed in Table 6. This testing is performed as follows. Clones are removed from the tissue culture vessels in which they were placed before the start of the calibrated stress conditions exposure (using methods appropriate for the cell type), and subdivided into 4 different aliquots. Each aliquot is re-plated in an appropriate tissue culture vessel in medium containing the following. For one aliquot, the medium is growth medium (conditioned medium may be used) only; for a second aliquot the medium is supplemented with guanosine or folic acid at a concentration sufficient to enhance the survival and expansion of HPRT⁻ clones; for a third aliquot, the medium contains the same level of agent as used for the calibrated stress condition exposure, and the fourth aliquot is placed in HAT-medium containing an amount of aminopterin that is sufficient to induce growth arrest and/or cell death in Phen-HPRT⁻ cells of the cell type of interest (see above). Cell morphology (where informative for cell behavior and/or gene expression) and cell growth are monitored over the next 2, to 4 days to determine the response of the cells to these differing conditions. Cell clones are classified according to the results as shown in Table 6. Cell clones can be subjected to additional testing as desired to evaluate further endpoints of interest and/or can be cryopreserved for future evaluations.

For some cell types and stress response induction agents/conditions, the number of surviving cell clones will be very large, precluding evaluation of all of the clones. In these cases, clones are numbered as they appear, and a representative portion of the clones is selected randomly for further testing. Then the frequency of appearance of each clone subtype is calculated for the entire surviving group of cell clones. In carrying out the process of selecting a representative subgroup of clones, it is advisable to number the clones and then to use a table of random numbers to select a representative proportion of clones that appeared early, moderately late, and late (or near the end) of this phase of the assay. This precaution is necessary because the timing of clone appearance and growth rate may reflect the level of damage in the cells of the clone and/or may reflect the level of damage that was resolved, using damage avoidance or damage tolerance pathways, before the cells re-entered the growth cycle.

This testing phase may identify other endpoints that can be used to further sub-classify clones expressing the general phenotype of interest. As an example, some cell lines express focus-forming morphology and characteristics following induction and resolution of some types of stress responses. If this type of a response is observed, surviving clones can be sub-classified into more sub-groups than indicated in Table 6. Thus, Table 6 provides an initial guide for clone sub-classification but it is not indicative of the only classification categories that can be used or that are informative of cell stress response resolution pathway expression and functionality. Additional classification categories that are discovered during the use and application of the SR assay are incorporated herein.

Phase 3 Step 2: Testing Phen-Alt Clones for Reversibility/Irreversibility of Phenotype

Cell clones sub-classified as Phen-alt clones require additional testing to be sub-classified further into Phen-alt-rev or Phen-alt-irr groups.

To subgroup Phen-ah clones as Phen-alt-rev or Phen-alt-irr, a representative group of these clones is tested further by subjecting the clones to repeated rounds of exposure to the calibrated stress conditions. Other stress inducing agents also may be used. Each exposure cycle is followed by a period of time to allow the cells to recover, and thus, to resolve the stress environment. After each exposure round, the percentage of cells that are resistant to the stress inducing agent or condition is recorded. Cell clone responses are monitored for one of two responses: (1) cell clone resistance to the effects of the stress-inducing agent increases with repeated exposures until the cell population expresses 100% resistance to the SR inducing agent (subclassified as Phen-alt-irr clones), or (2) the cell population does not show a significant change in resistance to the SR inducing agent and reverts to a parental cell response to the calibrated stress conditions agent after a period of time has elapsed (sub-classified as Phen-alt-rev clones). The parental cell response that has been used in the examples below is defined as greater than 99% sensitivity to the calibrated stress conditions agent or condition, as evidenced by cell cycle arrest and/or cell death following exposure to the SR induction agent. Other stress response endpoints may be used and will depend upon the nature of the stress inducing agent used; these endpoints are incorporated herein.

Phase 3, Step 3: Calculation of fCEs for Each Phenotypic Subgroup. These Calculations are Carried Out Using the Formulas Provided Below (See Specific Methods).

Phase 3, Step 4: Stress Response Resolution Pathway Evaluations

Total pro-survival pathway function: Total stress resolution pathway assessments are obtained by totaling the fCEs for all surviving clones, without regard for phenotype. Conversely, all raw data for surviving clones can be totaled and used in the fCE formula below to obtain and fCE for total pro-survival pathway function. This assessment then can be used to make comparisons between groups of cells evaluated in the SR assay.

Damage avoidance paths: The expression and functionality of damage avoidance stress resolution pathways is quantified using the fCEs of clones classified as Phen-parental. These clones are classified based upon the results of the series of tests presented in Table 7 and based upon the expression of wild-type or parental cell characteristics for all of the criteria presented in Table 6. The fCEs of these clones are defined in the SR assay as providing a quantitative measure of damage avoidance pro-survival pathway function. Specific methods for carrying out the testing listed in Table 7 are provided below, it is believed that there are little to no data about transient epigenetic modifications that these cells may undergo as part of the process of expressing damage avoidance paths and surviving the stress challenge. However, transient or persistent changes in the epigenome, chromatin conformation and/or structure may be identified in the future that are indicative of cells undergoing this type of stress response resolution; those changes then could be used as biomarkers of this response and to quantify the number of cells expressing this response type. Investigation of such epigenetic and chromatin modifications for use as biomarkers is planned as part of the future development of the SR assay. The use of such biomarkers for the purpose of identifying and quantifying cells expressing and/or utilizing damage avoidance paths to resolve the stress environment phase of the SR assay is incorporated herein. Other molecular markers also may be discovered in the future that are informative of the function of damage avoidance pathways and these are incorporated here as well.

Damage tolerance paths: the expression and functionality of damage tolerance stress resolution paths is quantified by determining the fCEs of Phen-HPRT⁻ mutant clones, PIG-A mutant clones (if determined), clones identified as having one or more mutations at a gene-specific locus of interest, and clones designated as Phen-alt clones. HPRT mutations are informative for mutagenic mechanisms that confer a survival advantage upon affected cells when 6-TG is used as the stress inducing agent, PIG-A mutations are informative for DNA damage that is not related directly to cell survival, and Phen-alt clone fCES are informative for genomic alterations (in DNA or in the epigenome) that confer expression of an altered phenotype with changes in cell morphology and function but retention of survival capability.

Cell clones with HPRT mutations have a survival advantage in the SR assay only when agents such as 6-TG are used as the stress inducing agent. As a result, the SR assay evaluates mutagenesis in three potentially different ways when 6-TG is used as the calibrated stress conditions agent, and similar types of information can be derived using different stress inducing agents that confer a survival advantage upon cells with specific characteristics. Recent work supports the hypothesis that HPRT mutant clones may serve as a quantitative indicator of the expression of REV3-mediated error-prone DNA repair processes that make up the branch of post-replication repair referred to as TLS, and thus, may be informative for the expression of damage tolerance resolution pathways (Zhu et al. 2003, Li et al. 2002, Diaz et al. 2003). Similar associations also may be true for PIG-A gene mutations, but these associations remain to be demonstrated for this gene.

Phen-HPRT⁻ clones are placed under an energy stress as a result of their inability to salvage guanine and the resultant need to use the de novo purine biosynthesis pathway to convert inosine into guanine. c-Myc protein affects the expression of the purine biosynthesis pathway. Some immortalized cells over-express this gene product, and thus, cannot readily up-regulate the purine biosynthesis pathway. These factors (energy stress and c-Myc gene expression) can diminish Phen-HPRT⁻ cell clone viability and growth rates. In the SR assay, Phen-HPRT⁻ cell clones grow more slowly than other surviving cell clones and have increased rates of cell death, especially for clones that have been in culture for several weeks. These characteristics facilitate the identification of these clones in the SR assay. Once identified and isolated, a portion of each clone is grown in the presence of guanosine or folic acid to prevent loss of these clones during clone expansion.

Epigenetic modifications associated with induction or formation of mutations in the HPRT and PIG-A genes have been described in a few cases. As noted above for Phen-parental clones, transient or persistent changes in the epigenome may be identified in the future that are indicative of cells undergoing this type of stress response resolution and those changes may serve as biomarkers for surviving cell clones with mutations in HPRT, PIG-A, or other loci of interest. The use of such epigenetic alterations for the purpose of identifying and quantifying Phen-HPRT⁻ cell clones and/or clones with imitations in PIG-A or at other gene specific loci is incorporated herein. Other molecular markers of Phen-HPRT⁻ clones may be identified in the future and these are incorporated here.

Damage tol-rev and damage tol-irr pathways: The total expression of damage tol-irr pathways in the SR assay is considered to be equal to the sum of fCEs of Phen-HPRT⁻ clones and Phen-alt-irr clones. PIG-A mutant clones and clones shown to have mutations in other genes of interest also can be included in these groups if available. Mutations in DNA currently are considered to be irreversible forms of genomic damage, but it is believed that some of these mutations may be reversible through repair or elimination of affected cells from the cell population. Thus, under some circumstances, clones with gene-specific mutations may not be included in the determination of the total frequency of damage tol-irr path expression.

Pathway potency measurements can be made by dividing the total fCEs for damage avoidance or damage tolerance pathways by the SRID-x % level (expressed using dose units of choice) used in the assay. This evaluation provides an ‘effect per unit dose’ for each pathway, and also can be applied to total pro-survival pathway function by using the total fCEs for all SR assay surviving clones. This type of assessment may be particularly useful for comparing chemical or drug effects or for making comparisons between cell lines or cell types.

Phase 3, step 5: Assessment of final surviving cell population(s) composition and damage burden: The above described methods for determination of fCEs are based upon assessment of surviving clones as fractions of the original, tested cell population. However, it is important in the SR assay also to assess the surviving clones as fractions of the total cell populations that survived in the SR assay. The primary goal of these assessments is to determine changes in the damage burden of SR assay surviving cell populations, for comparisons between different populations and for comparisons to the damage burden in one population before and after calibrated stress conditions exposure. See example #1 for an example of this type of evaluation. This type of assessment may be more informative for deleterious effects of some exogenous agents because it makes it possible to see how the characteristics of the surviving cell population have changed, and takes into account effects of a stress environment upon total cell survival, which when combined with effects upon cellular damage burden, result in significant changes in the nature of the surviving cell population that may be related to disease risk. The total clone counts for each phenotypically distinct clone type, and total clone counts for all surviving clones are used to determine the percentages that each clone phenotype represents out of the total number of surviving clones. Phen-parental clones are used as indicative of surviving clones with no evidence of alteration in their damage burden, and Phen-HPRT⁻ clones and Phen-alt-irr clones are used as indicators of permanent increases in damage burden. Phen-alt-rev clones are considered to be ambiguous at this time and can be classified in either group. The change in total damage burden for each cell population is determined and comparisons between populations can be made. As an alternative, fCEs based upon the total surviving cell population clones can be calculated as another method for describing the composition of the final surviving cell population (also see example #1).

Phase 3 Step 6: Additional Assessments Based Upon SR Assay Data and/or SR Assay Derived Materials

Determination of PIG-A mutation frequencies or determination of mutant frequencies at one or more gene specific loci:

DNA damage levels are measured by determining the frequency of clones with mutations in the HPRT and/or PIG-A genes. In addition, DNA damage can be assessed by evaluation of mutations at any reporter gene or other gene-specific loci, using available methods as appropriate for those goals. Gene sequencing can be used to characterize further the number and types of mutations and can be used to confirm that mutant cells have mutations at the locus of interest.

Determination of post-SR induction PIG-A mutant frequencies represents an additional method that can be used to describe damage avoidance versus damage tolerance outcomes of the stress resolution pathways evaluated in the SR assay. PIG-A mutant frequencies can be determined for pooled surviving cell clones from each subgroup and also can be determined for all surviving cells as a single group. This latter determination allows comparisons to be made to the PIGA mutant frequencies of the entire surviving cell population of interest prior to induction of a stress environment and after induction and resolution of the stress environment. It also allows comparisons of the total or overall mutational burden in vehicle-exposed or control cells versus cells that have been exposed to a test agent. Unlike the case for HPRT, no evidence is available to determine if mutations in the PIG-A gene also are indicative of expression of damage tolerance stress resolution pathways including TLS pathways.

PIG-A mutant frequency determination both before and after the calibrated stress conditions step of the assay can be used as a quantitative measure of the expression and/or functionality of damage avoidance paths versus damage tolerance stress resolution paths, and to determine if pre-exposure to an agent or condition affected the expression and/or functionality of these pathways. Determination of PIG-A mutant frequencies both pre- and post-calibrated stress conditions exposure will make it possible to test the hypothesis that stress response resolution is sufficiently robust to resolve stress-induced damage with a reduction in the spontaneous or existing mutational burden, thereby conferring a beneficial effect.

Determination of Tumorigenicity of SR Assay-Derived Clones:

Assessment of the an additional type of the cell population damage can be made by determination of the tumorigenicity of SR assay surviving clones, especially Phen-alt-irr clones, using parental cells and/or other clones as controls. Determination of tumorigenicity serves as a quantitative indicator of the occurrence of cells with irreversible damage to critical cell systems necessary to prevent expression of the tumorigenic phenotype. Currently these systems are hypothesized to include cell-cell communication systems, apoptotic systems (both infra- and intercellular apoptotic pathways), systems involved in the expression of genomic instability, and systems responsible for maintenance of ECM. Tumorigenicity can be assessed in vivo using nude mice or in vitro using soft agar assays including high throughput variations on soft agar assays. It is possible that additional work will identify other cell systems that must be altered in order for a tumorigenic phenotype to be expressed. Determination of tumorigenicity provides a disease-related endpoint for the SR assay. Most importantly, the potential for agents or conditions to increase or decrease the tumorigenic capacity of cells currently is tested in rodents and extrapolation of results to humans is associated with varying levels of uncertainty. The SR assay will allow direct testing of tumorigenicity in human cells of interest, thereby reducing this uncertainty.

SR assay surviving clones with the Phen-alt-irr phenotype have been found previously to be tumorigenic when tested in nude mice (see example #8 below). Any of the subgroups of surviving cell clones can be tested for tumorigenicity by injecting cells into immuno-compromised mice or other rodents, or by testing the cells for expression of the tumorigenic phenotype using a soft agar assay or in vitro using soft agar assays including high throughput variations on soft agar assays. In the examples given below, tumorigenicity was determined for mouse cells exposed to a prolonged, high-level glucose deprivation stress challenge combined with exposure to cell crowding stress. Tumor frequency was determined as a function of positive versus negative injection sites; tumor latency was determined as the time from inoculation of cells to the appearance of a 5 millimeter or greater sized tumor. The results showed that this high-level stress environment resulted in significantly increased tumor frequency and significantly reduced tumor latency when compared to cells that were not exposed to the stress environment. It is possible that additional work will identify other cell systems that must be altered in order for a tumorigenic phenotype to be expressed. As described above, additional molecular, DNA or epigenetic markers are expected to be identified through future studies that are indicative of transformed cells and that will facilitate the identification of clones that express a tumorigenic phenotype. The use of such biomarkers for the purpose of identifying and characterizing these cells and clones is incorporated herein. Future work is anticipated to discover biomarkers or molecular changes in cells that correlate with the tumorigenic potential of the cells. These endpoints or biomarkers are incorporated herein.

Assessment of epigenetic and/or chromatin alterations as indicative of damage tolerance versus damage avoidance pathway outcomes.

Epigenetic alterations, gene expression changes, and chromatin conformation changes currently are evaluated by assessment of changes in the parental cell characteristics presented in Table 6, and by evaluation of the cells for changes in function, including loss of barrier function and cell cycle checkpoint arrest capacity. In addition, the ability of the cells to respond to other stress-inducing conditions or agents also may be used as part of the assessment of cell function. As indicated above, molecular markers for post-calibrated stress conditions exposure determination of epigenetic alterations are expected to be developed through future investigation of the SR assay and these markers are incorporated herein.

Data Comparison Assessments

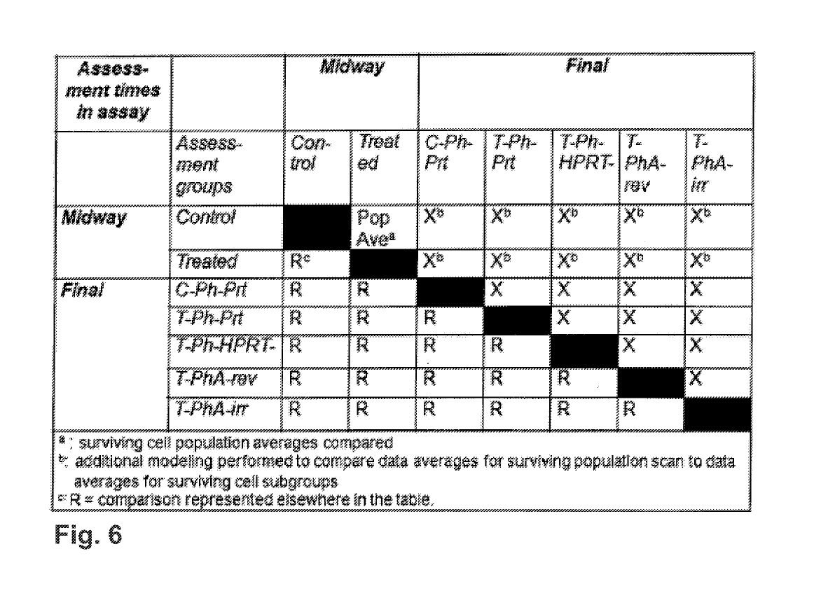

Comparisons are performed for the purpose of evaluating SR assay data. FIG. 6 provides an example of the types of comparisons that can be made with SR assay data. First, by comparing endpoint values determined before versus after the calibrated stress conditions exposure step, it is possible to derive information about the impact of stress response resolution upon the status of SR assay outcomes and to draw conclusions about the types of stress resolution pathways that have been expressed by differing populations of cells during the stress resolution process. Comparisons can be drawn between results for one cell population, before and after calibrated stress conditions exposure, and between two different cell lines or populations. Current data obtained from the SR assay support the postulate that normal cells with robust stress response resolution capacity (good damage avoidance pathway capacity) have few to no differences in cell population characteristics as measured before versus after the calibrated stress conditions, a finding consistent with the expression of damage avoidance stress resolution pathways to resolve the assay-induced stress environment. However, cell populations with weak damage avoidance stress response resolution capacity and/or a significant damage burden have identifiable changes in cell population characteristics when measured before versus after SR induction.

Comparisons also can be made between two cell populations to compare and contrast stress response resolution pathway expression and functionality for the purpose of determining differences between cell populations. This type of comparison especially is useful for experiments designed to determine the effects of an exogenous exposure upon stress resolution pathway expression and function. Since comparisons of SR assay-derived data have not been possible prior to the development of the SR assay, it is probable that other methods and/or applications for these comparisons will be identified through future work. Any known methods can be applied to enhance the information derived from these comparisons. In addition, methods developed in the future, if applied to SR assay-derived data and/or outcomes, are incorporated herein.

Determination of the fCEs for Phen-alt-irr clones can be used as an additional point of comparison for two cell populations, such as control versus treated cell populations, to determine if a representative cellular process (such as ECM, maintenance of genome stability) has been damaged so that its function is lost, and/or to determine if cellular transformation with expression of a tumorigenic phenotype has occurred. In addition, future development of the SR assay will make it possible to identify other SR assay endpoints and to define further the significance of these endpoints. These to-be-developed endpoints and methods for their assessment are incorporated herein.

Other Assessments Using SR Assay Derived Data and/or Materials

In general, any endpoints of interest can be evaluated, in addition to the fCEs calculated using SR assay binomial data, as part of the final SR assay assessments. Endpoints that have been evaluated post-calibrated stress conditions exposure and resolution include (1) determination of the phenotypes and fCEs of SR assay surviving cell clones, (2) determination of the functionality of stress resolution pathways by using the fCEs of phenotypically distinct clones as indicators of total pro-survival pathway function, damage avoidance pathway function, and damage tolerance pathway function, (3) determination of the damage burden in the cell population(s) that survived in the SR assay, (4) determination of expression of a tumorigenic phenotype for clones of interest, (5) assessment of comparisons between clones (see FIG. 6), and (6) assessment of any other endpoints of interest. Other assessments of cell function may be included in the SR assay at a future time for the purpose of further evaluating stress response resolution pathway expression and function, and are incorporated herein.

The fCEs for Phen-alt-irr clones provides a mechanism for determining if genomic damage is sufficient to affect one or more critical cell functions, with ECM function being one example. Thus, quantification of the frequency of occurrence of Phen-alt-irr clones in the SR assay surviving cell population is informative for the presence of surviving cell clones with high levels of damage that are of sufficient magnitude to compromise the functionality of other damage-sensitive cell systems and processes.

The fCEs of Phen-alt clones also can be used as indicative of a transient or persistent period of genomic instability, which is hypothesized by several investigators to be a prerequisite for achieving high mutational or damage levels. In the SR assay, the functionality of ECM-related processes is used as a biomarker for retention or loss of functionality of other cellular processes/systems that are known or hypothesized to be critical for resistance to or sensitivity to disease occurrence, including transient or persistent genomic instability. Little is known about the types of changes or damage that alter the functionality of ECM maintenance and restoration systems. The SR assay described herein provides a mechanism for investigating these phenomena. Future investigations are expected to result in an increase in knowledge related to the ECM systems, processes, and elements involved in their functionality. The molecular basis for the delay and/or failure of ECM restoration may be determined through future research, so that biomarkers indicative of damage to this system will be identified and can be used to assess the functionality of this system and effects of exposures upon this system. These molecular changes and biomarkers are incorporated herein.

The fCEs of Phen-alt clones can be used quantitative indicators of the expression and function of pathways associated with checkpoint adaptation (see definitions). Phen-alt clones have very long latency periods and emerge from a background of cells that appear to be senescent or dead. These observations support the conclusion that Phen-alt clones can be used as indicative of checkpoint adaptation as an example of one type of damage tolerance mechanism.

The SR assay described herein can be used to identify additional endpoints that are indicative of and/or can be used as biomarkers for the differing types of stress resolution pathways that can be expressed by cells. Epigenetic alterations, molecular markers, or gene expression profiles indicative of expression of particular stress resolution pathways may be described in the future. These markers then could be used as biomarkers for expression of that pathway(s) and could replace the phenotypic testing currently used to sub-group surviving cell clones. These novel biomarkers are incorporated herein.

Specific Methods Used to Complete the Phases and Steps of the Assay

The SR assay described herein has been used to evaluate stress response resolution pathway expression and efficiency in cells following induction of a short-term, moderately intense stress environment. The methods presented below describe the procedures necessary to complete the assay for the evaluation of these types of stress responses.

Cell Culture

The cells of interest are grown according to conditions previously shown to be optimal for their growth and survival, and these conditions will vary depending upon the cell type or cell line selected for use. Cell culture conditions that result in less than optimal growth rates and/or reduced cell viability should not be used as they may introduce confounding factors, especially other stress responses that may affect the assay results.

Several components used in the cell culture medium during the conduct of the assay are important and can affect assay results. To date, all cells have been grown in medium that contained serum as these conditions have been found to enhance recovery of surviving cells under stress.

Cell Immortalization Techniques

A variety of methods to immortalize primary cells for the purpose of allowing them to be cultured in vitro for periods of time that exceed those of the primary cell(s) have been described in the literature. These techniques include but are not limited to use of the SV40 antigen, infection with human papilloma virus or components thereof or papilloma viruses appropriate for the species from which the cell type of interest is obtained, use of a hTERT plasmid with or without a cdc4 plasmid, and others. In addition, because immortalized cells that have retained as much normal function as possible continue to be needed in research, new cell immortalization techniques are being developed frequently. Existing and to-be-developed cell immortalization techniques are incorporated herein.

Exposure to Exogenous Agents (if Desired):

For evaluation of the effects of a pre-exposure to an agent or condition upon stress response resolution pathway expression and function, the cells should be exposed under conditions that are relevant for the agent or condition of interest. Since these are expected to vary considerably between different agents or conditions, a description of these conditions cannot be provided here. These exposures may induce expression of a stress response in the cells of interest; this stress response is not considered to be equivalent to the calibrated stress conditions exposure used as part of SR assay, and cannot be used as a substitute for this step in the SR assay.

Adaptive Response Period (Phenotypic Expression Period):

Following the exposure period, the cells may be washed to remove any agent or compound (if necessary). Cells are expanded in growth medium under optimal growth conditions for a period of time appropriate for the cells and the experimental design. One to two weeks, or for a period of time that allows for at least 2 population doublings to occur has been found to be adequate in the past for assessment of long term effects of test agents upon cellular stress response function. During this time period, proteins in the cell cytoplasm that accumulate prior to stress response induction or mutations in target genes or phenotypic alterations in chromatin are removed and/or replaced by proteins representative of mutated target genes or newly expressed genes, if present, in addition, genetic and phenotypic alterations will be fixed by additional rounds of replication and some expansion of cells with mutations and/or phenotypic alterations will have occurred. Other changes of interest also will be resolved and/or will become stable during this time period. In the examples given below, the cells were given a 2 week period for phenotypic expression, and this was equivalent to approximately 4 to 6 population doublings. Feeding with conditioned medium may be desirable and may improve recovery of surviving clones. It is especially important to keep cell density within optimal limits, since cell crowding can induce stress responses (termed ‘crowding stress’) and SR induction outside that used in phase 2 of the assay is to be avoided due to potential confounding effects. The phenotypic expression period must be adapted to the cells being used in the SR assay, and no one duration time for this phase of the assay can be described due to differences in cell growth rates and metabolism rates.

Methods for Plating for the Calibrated Stress Conditions Exposure:

At the end of the phenotypic expression period, the cells are removed from the tissue culture vessels using methodology appropriate for the cell type. The cells are pipetted repeatedly to break up any cell clumps and to render the cells into a single cell suspension. For cells that form tight junctions, and/or grow attached to each other or a basement membrane, filtration through sterilized Nytran filter material or other comparable filter material, small pore size (20-25 microns has been used) using gravitational forces without application of suction is recommended. The purpose of this step is to remove large cell clumps that cannot be reduced by repeated pipetting. Application of suction is undesirable because it can pull cell clumps through the filter material intact, especially for moderately to highly plastic cell types such as some epithelial cells, so that the goal of the filtering process is lost. A sample of the cell suspension is examined by light microscopy to determine that a near-uniform single cell suspension has been achieved. The cells are counted by hand or using an automated cell counter such as a Coulter counter, and diluted for plating on tissue culture vessels for exposure to the calibrated stress conditions. An aliquot of cells is removed and reserved for use in determining cell in CEs (see below). The cells are plated at the optimal density for calibrated stress conditions exposure as determined using the methods described above. Any tissue culture vessel can be used, but quantification of assay results is facilitated greatly if standard 48 or 96 well tissue culture plates are used. Tissue culture vessels with fewer or more wells per plate also can be used provided that the cell plating density is changed to take into consideration the size of the wells to be used.

Methods for Determination of Midway CEs (mCEs):

The aliquot of cells set aside (see above) for this purpose is used. Two procedures are described for determination of cloning efficiencies. The first has been described previously for use in the T-cell cloning assay, but its use in the SR assay as described herein is novel and is included herein. This procedure is recommended for cells that grow in suspension and/or do not have the characteristics described below for epithelial cells.

mCE Procedure #1 for Cells that Grow in Suspension and/or Lack Cell Growth Characteristics of Epithelial Cells

The cells are rendered into a single cell suspension and counted by hand or using an automated cell counting system or machine; then the cells are diluted to a low density of approximately 1 to 10 cells per 100 to 200 microliters, mixed to ensure uniform distribution, and pipetted into individual wells of tissue culture plates. These cells may be mixed with or plated along with ‘feeder’ cells to improve viability (‘feeder’ cells are cells that have been irradiated so that they cannot replicate, but they remain viable for a period of time that is sufficient to support the growth of the test cells). Following passage of the appropriate time interval, the wells are evaluated for colony growth and/or expansion. The number of positive wells is counted and the cloning efficiency is calculated using the formula provided above (see phase 3 step 5 above).

mCE procedure #2 for cells that grow attached to a surface or basement membrane and/or that have the following growth characteristics when grown in tissue culture. The second method is novel and is recommended for use with epithelial cells, cells that form tight junctions, cells that produce mucinous material during in vitro cell culture, and/or cells that attach to a basement membrane. The latter characteristics are typical of epithelial cells but are not unique to this cell type alone.

This procedure is used to obtain an accurate cell count in vials of very dilute cells that are used for plating cloning efficiency plates. Small but significant errors can be introduced during the dilution of the cells and the occurrence of these errors is enhanced in suspensions of epithelial cells and cells with epithelial-like characteristics due to their tendency to stick or attach to each other or to mucinous material, even after filtration for the purpose of producing a single cell suspension. These errors can have a significant impact upon the determination of the plating efficiency. The following method is intended to resolve this problem by determining more precisely the number of cells in the dilutions that are plated for measuring cloning efficiencies.

For this procedure, the cell aliquot that was set aside during the initial cell counting is divided into 2 equal groups and diluted as follows. Group A cells are diluted to a concentration of 50 cells per milliliter (1 cell per 20 microliters), and group B cells are diluted to a concentration of 5 cells per milliliter (or 1 cell per 200 microliters). Group A cells are plated at the rate of 200 microliters per well on two (or more) 96 well plates, and at the rate of 1 milliliter per well into 3 to 6 wells of 6 or 12 well plates. Group B cells are plated in the same way. Approximately 12 to 15 hours later, the average number of cells per milliliter on the 6 or 12 well plates for both groups A and B is determined using light microscopy to visualize and count the cells. All cells are counted, including both viable and dead cells. A supravital dye may be used if it makes cell visualization and counting easier. These counts then are used to determine with the greatest degree of accuracy the concentration per milliliter of the cells in groups A and B. One of these 2 groups will be closer to a concentration of 5 to 20 cells per milliliter than the other. Because this is the optimal plating density for determining cloning efficiency with epithelial cells and cells with epithelial-like growth characteristics, the 96 well plates belonging to that group then are used for the determination of cloning efficiencies, and the 96 well plates belonging to the other group are discarded. Viable cells are counted; dead cells or cell fragments are not counted. This counting procedure should be completed within 2.4 to 36 hours after the plating is completed, or before one population doubling has occurred. Waiting longer will result in death of some cells and replication of others causing the cloning efficiency counts to be inaccurate. The final cloning efficiency is determined using the formula presented above (see phase 3 step 5). Any tissue culture vessel(s) can be used, as long as the general principles described above are adhered to and can be achieved. In addition, supravital dyes may be used to enhance the ability to visualize the cells. This method for determining the cloning or plating efficiency of epithelial cells and cells with epithelial-like characteristics is incorporated herein.

Calibrated Stress Conditions Exposure:

The specific methods for completing this process have been described above.

Identification and quantification of surviving, presumptive Phen-HPRT⁻ clones. Determination of the mutation frequency at the HEIST locus (determination of Phen-HPRT⁻ CEs) is recommended but is not a required component of the assay. Phen-HPRT⁻ clones can be visualized after the induction of a stress response shortly after the end of the calibrated stress conditions exposure step of the assay. Starting shortly after the end of the calibrated stress conditions exposure step, and as soon as dead cells have lifted off the plates and the appearance of cells remaining in the wells is stable, all wells are evaluated for evidence of small cell clusters with normal to near normal log phase growth morphology. These clusters usually are visible against a background of cells with morphology compatible with cell death or stationary phase. The exact period of time between the cessation of the calibrated stress conditions exposure step and the optimum time period for visualizing the small cell clusters will vary depending upon the cell type used in the assay and also the stress inducing agent; however, in the examples given below the optimum time was approximately 7 to 10 days after the cessation of the assay-associated stress environment and extended out for approximately 14 to 20 days. All wells containing small cell clusters should be marked for future evaluation as presumptive Phen-HPRT⁻ clones. These clones should be followed over a several day period in order to determine the growth rate of the clones; cell clusters showing slow growth rate have a high probability of being Phen-HPRT⁻ clones.

Clones with a presumptive HPRT⁻ phenotype are characterized using the phenotypic tests described (see Table 7) and/or by sequencing of the HPRT gene for evidence of mutations. It is possible that cells with other mutations that confer a survival advantage also will be isolated along with HPRT⁻ clones and the frequency of survival of these clones also may be quantified as part of the SR assay. Quantification of these clones is incorporated herein.

Identification and Quantification of Surviving Phen-Parental Clones

Phen-parental clones appear around the same time or shortly after Phen=HPRT⁻ clones can be visualized, and the appearance of these two groups of clones may coincide. Phen-parental clones are recognized as described above. These clones are counted as they appear and their appearance can be characterized as early, moderately late and late if desired.

Identification and Quantification of Phen-Alt Clones

The time period for initial identification of clones with phenotypic alterations of interest varies depending upon the cell types being evaluated and the type and intensity of the calibrated stress conditions used in the SR assay. In the examples given below, this period began approximately 10 to 14 days after the cessation of calibrated stress conditions exposure and extended out for five to six weeks post-calibrated stress conditions exposure. When 6-TG was used as the SR inducing agent and survival was used as the phenotype of interest, cell clones arose from a background of cells that appeared dead or to be stalled in stationary phase. All wells containing such clones should be determined; it frequently is necessary to evaluate the plates multiple times to determine that a final correct count of viable clones has been obtained because clones can arise over an extended time period, and recording of the date of appearance of the clones is advisable.

For some studies it may be advisable to record the time period in which clones with the phenotype of interest are identified. For example, in studies in which the induced stress response is a DNA damage response and the phenotype of interest is survival, surviving clones can be identified as early as a few days after cessation of the calibrated stress conditions and up to six weeks later. Current results from the SR assay support the conclusion that there are significant differences in the types of alterations in these clones, and that severity or the extent of the damage and/or alterations that have occurred during stress response resolution correlates with the length of time that elapsed between the calibrated stress conditions exposure and clone identification. Timing of clone appearance, clone growth rate, and phenotypic testing are used to sub-group the surviving cell clones.

Methods for Performing Calculations Used in the Assay:

Method for Calculating ‘X %’ as Used in the SRID-X % Level of Calibrated Stress Conditions Agent:

X=(total negative wells/total wells)×100%

Method for Calculating the Total and Subgroup Final CEs (fCES):

$$fCE = \frac{( - ln ( total \# negative {wells}^{a} / total \# of wells )}{( \# of cells plated per well \times mCE )}$$

Method for Calculating the Midway CEs (mCEs):

$$mCE = \frac{( - ln ( total \# negative {wells}^{a} / total \# of wells ) )}{\# cells plated per well}$$

Methods for Calculating Pathway Potency Assessments

$$Pathway potency = \frac{fCE ( for pathway of interest )}{\begin{matrix}dose level of calibrated stress inducing \\ agent ( expressed in any appropriate units )\end{matrix}}$$

Determination of Genetic Alterations at Loci Other than the HPRT Gene: PIG-A Mutant Frequencies.

Mutagenesis at the PIG-A locus or any other locus of interest that can be assessed without exposure of cells to a stress environment, such as determination using a flow cytometric method, is incorporated herein.

FACS Assay of epithelial cells for PIG-A mutations. Epithelial cells of diverse types and/or origins express GPI-anchored proteins, but the nature of the proteins varies with cell type and species. Thus, it is necessary to determine the identity of GPI-anchored proteins of interest for use in determination of PIG-A mutant frequencies for each cell type of interest. Additional modifications of the methods provided below will be needed to adapt these techniques for use with epithelial and epithelial-like cells and to address the issue of autofluorescence that characterizes epithelial cells and epithelial-like cells (see below). Techniques for addressing the issues are available in the literature and are incorporated herein.

FACS Assay of Lymphocytes for PIG-A mutations. The following procedure has been used independent of the SR assay and has been presented in the literature. The use of this assay as a component of the SR assay described herein for application to SR assay-derived clones is novel and is incorporated herein. The assay described briefly below is applicable to the analysis of lymphocytes in whole blood or washed lymphocytes. Two aliquots of 1.5 ml of whole blood (or washed lymphocytes) are prepared. Fifteen microliters of a pre-defined mixture of anti-CD3APC, anti-CD4 PerCP-Cy5.5 and anti-CD8 PE is added to one of the 1.5 ml aliquots of whole blood (or washed lymphocytes) and incubated at 18° C.-26° C. in the dark for 15.0 min. The second aliquot is left unstained. Eight ml of FACS Lysing Solution (prepared per manufacturer's instructions) is added to both 1.5 ml aliquots of cells (i.e., of ‘stained’ whole blood/lymphocytes and of unstained cells), both are vortexed briefly and incubated with rotation (15 min, 18° C.-26° C. dark). Both samples are vortexed briefly, centrifuged (300×g, 5 minutes), and then the supernatant is removed. Cells from both aliquots then are washed once with PBS 0.10% azide pH 7.4 and centrifuged (300×g, 5 min). The supernatant of each aliquot is aspirated, leaving ˜400 μl remaining for each. Twenty μl of a combination of anti-CD55 FITC and anti-CD59 FITC is added to the washed cells in the aliquot previously stained with anti-CD3. These multi-stained cells then are incubated at 18° C. −26° C. for 15 min and stored at 4° C. until cytometric analysis. The 400 μl remaining in the unstained aliquot of cells then are distributed to five 50 μl fractions. Four of these fractions are stained with a single (or double for CD55+59) labeled antibody each, i.e. anti-CD3, anti-CD4, anti-CD8 or anti-CD55+59, while the fifth fraction is left unstained. These five fractions are used to establish gates used in the flow cytometric analysis of the cells. FACS analysis of lymphocytes is performed using the BD LSRII flow cytometer with BD FACSDIVA software version 4.12. This instrument is equipped with a 488 nm coherent Safire laser and a 633 nm JDS uniphase hene laser.

Molecular Analyses of PIG-A mutant T-Cell Isolates: RNA or mRNA isolated by any available technique can be used for these analyses. The RNA or mRNA also can be snap frozen and stored at −80° C. for later use. Because of the relatively large size of the PIG-A coding sequence (1455 bp), it is advisable to sequence it in two pieces. This sequencing can be performed as follows: the 3′ part of the gene is sequenced by RT-PCR (using a sense primer in exon 2 and an antisense primer 3′ of the gene). The 5′ part of the gene is sequenced either by RT-PCR using a sense primer in exon 1 (exon 1 is non-coding) and an antisense primer in exon 2 such that there is overlap of the 5′ and 3′ sequences in exon 2 or by genomic sequencing of exon 2 (exon 2 is very large, 777 bp). Any other sequencing techniques and/or procedures also can be used. Genomic amplification and sequencing uses the isolated “mRNA” Which actually contains a quantity of genomic DNA. Both methods miss sequencing exon 1 which is essentially only a splice junction. Exon 1 is amplified and sequenced separately if no mutations are found in exons 2 to 6. For those mutants not producing cDNA, a multiplex genomic PCR is performed for exons 1 (202 bp amplicon), 2 (892 bp), 3 (244 bp), 4-5 (671 bp together) and 6 (470 bp) to check for genomic deletions.

Future studies probably will identify other molecular or gene targets that can be used to measure the presence of mutations either pre-SR induction, post-SR induction, or both. In addition, future developments of the PIG-A mutation assay also are expected to be reported. These developments may result in significant alterations in the techniques for measuring mutagenesis at the PIG-A locus. These future developments and/or targets are incorporated herein.

Methods for Determination of Tumorigenicity:

Evaluation of tumorigenicity can be used to determine if exposure of a cell population to a test agent or condition, followed by induction and resolution of a stress response, has altered the tumorigenic potential of that cell population. Surviving cell clones identified and/or isolated post-calibrated stress conditions exposure and resolution, and especially those clones classified as Phen-alt-irr, are used for determination of post-SR induction tumorigenicity.

It is recognized that many tumorigenic human cells and cell lines will not grow in nude mice, SCID mice, or other immuno-compromised mice. In addition, testing for tumorigenicity in rodents is time consuming, expensive, and may give ambiguous results or false negative results due to the difficulty of growing human cells in rodents. As a result, the preferred method for testing for expression of a tumorigenic phenotype by clones isolated using the SR assay is to use the soft agar assay. Recently, an automated high throughput soft agar colony formation assay has been described with methods (Thierback and Steinberg 2009). The soft agar colony formation assay and variations and/or developments of this assay, including but not limited to future developments, are incorporated herein.

An alternative method for assessment of tumorigenicity is as follows. Cells demonstrated to have or presumed to have the characteristics of Phen-alt-irr cell clones are injected into a fat pad, usually the subscapular or mammary fat pad of immuno-compromised mice, including but not limited to nude mice or SCID mice, at the rate of 3×10⁶ to 5×10⁶ cells per injection site. Cells of the same type that have not been subjected to a stress challenge, and surviving cell clones, classified as Phen-parental, serve as controls and should be injected in the same way and at the same cell concentrations. Other SR-assay derived surviving cell clones also can be used. Injection sites are monitored for a sufficient period to determine the tumorigenic frequency, expressed as the ratio of positive injection sites versus total injection sites, and the latency, expressed as the time of injection to the time of appearance of the first tumors. If Phen-alt-irr surviving clones are determined through further investigation to have a tumorigenic phenotype, then their appearance in the SR assay will be evidence of the ability of a stress challenge, with or without a pre-exposure, to induce expression of the tumorigenic phenotype. When used in the context of evaluating the effects of a pre-exposure to an agent or condition upon cell function, the appearance of Phen-alt-irr clones supports the characterization of the agent or condition as one with tumorigenic potential in the cell type tested. Both of the above methods of testing for expression of a tumorigenic phenotype (soft agar assay and its variations and injection into immuno-compromised mice or other rodents) are incorporated herein.

Future work is expected to reveal both genetic and epigenetic alterations that can be used as biomarkers for the expression of a tumorigenic phenotype. The identification of such markers will facilitate classification of cell clones as to their tumorigenic potential and may reduce or eliminate the need for testing in animals or using the soft agar assay or comparable assays. The use of such molecular markers of tumorigenicity that are discovered in the future are incorporated herein.

Preparation and Use of Hat-Medium:

A stock solution of thymidine, hypoxanthine and cytidine (THC) is made up with cytidine: 24.3 mg/1.00 ads; hypoxanthine: 272 mgs/100 mls; thymidine; 42.4 mg/100 mls. These reagents are dissolved in 0.1N NaOH until completely in solution; then the solution is brought up to 100 mils with deionized sterile water. Aminopterin is made up at 0.1 mg/ml in 0.1N NaOH. One milliliter of THC stock solution is used per 100 mls of growth medium along with 100-400 microliters of aminopterin per 100 mls of medium. The pH of the HAT-should be adjusted to 7.3 and filter-sterilized before use. The amount of aminopterin to be used depends upon the cell type and should be tested independently before use. The maximum amount of aminopterin that wild-type cells can tolerate without visible effects should be used.

Preparation and Use of Conditioned Medium:

The conditioned medium used in these examples was prepared as follows and similar procedures can be used for the preparation of conditioned medium to be used in the SR assay. Newly prepared growth medium appropriate for the cells in log phase growth is applied to the cells on day one; 24 hours later the medium is removed and its volume is estimated; sufficient fresh fetal bovine serum is added to the medium in an amount that is equal to 10% of the estimated volume of the removed medium; the conditioned medium is mixed briefly, and filtered through a 0.45 micron pore size filter for the purposes of removing any viable, suspended cells. The conditioned medium is then mixed in a 1:1 ratio with newly prepared growth medium and applied to the cells. Usually, the cells in log phase growth used for the preparation of conditioned medium should be the same cell type as the cells that will be fed with the conditioned medium; however, other cell types can be used and may, in some circumstances, provided improved cell growth and/or survival. The period of conditioning may vary, but 24 hours has been found to be effective in the examples given below. Other methods for preparing conditioned medium can be used and are incorporated herein.

Methods Used to Assess Epigenetic and/or Chromatin Modifications:

A list of methods reported in the literature is provided in Table 9. These methods differ in the types of information that they provide and they are not all equally valuable for use in evaluating SR assay clones. Since the types of epigenetic modifications that are detected using the SR assay remain to be described, it is not possible to determine which of these techniques will be most useful. In addition, the types of epigenetic modifications vary by cell type and probably also by the type of stress environment, so no one method can be described for assessment of all epigenetic alterations that may be of importance in the SR assay. New analytical techniques for assessment of epigenetic modifications are being described in the literature, due in part to the increasing recognition of the role of epigenetic changes in health and disease. Thus, currently existing techniques for assessment of epigenetic modifications in human and animal cells, as well as techniques developed in the future for the same purpose, are incorporated herein.

Methods used to assess gene expression changes: any currently available methods for assessment of gene expression profiles can be applied in the SR assay. Methods developed in the future also can be applied and are incorporated here.

Methods used to assess microRNAs: any currently available methods for assessment of gene expression profiles can be applied in the SR assay and are incorporated here.

Methods used to assess mitochondria and mitochondrial function: any currently available methods for assessment of gene expression profiles can be applied in the SR assay and are incorporated here.

### DEFINITIONS

As used herein, each of the following terms has the meaning associated with it in this section.

Adaptive responses or pathways are pathways used by cells to respond to and to adapt to stress conditions. The goal of these responses is to protect the cell, to repair damage, and to maintain or to restore homeostasis. Some investigators appear to use ‘adaptive responses’ to mean or imply damage avoidance pathways.

Adaptive gene amplification is a mutagenic process associated with untargeted mutagenesis. As an example, adaptive gene amplification in E. coli requires RpoS (a transcriptional activator of stationary-phase/starvation- and general-stress-response-specific genes and that also is required for point mutation) and DNA polymerase I (not involved in adaptive point mutation). It is believed that mutant cells with adaptive gene amplifications do not arise from a hypermutable subpopulation of cells with amplification of unrelated genes. Adaptive gene amplification does not require polymerase IV or induction of other SOS proteins; some recombination proteins are involved in the response. Adaptive gene amplification also is a phenomenon that is currently undergoing active investigation by a number of laboratories in the United States and in other countries; these investigations are expected to describe additional characteristics and/or genes involved in the process in the future. Adaptive gene amplification has been studied most using microbial models and less in known about the phenomenon in human and animal cells.

Adaptive point mutation is a mutagenic process associated with untargeted mutagenesis. One example is the Lac frameshift system of E. coli; this example is considered to be one of the best understood in terms of the mutation mechanisms involved. Lac+ point mutations occur during stationary phase: mutagenesis in this system differs from targeted mutagenesis in that, in microbes, it requires the recombination proteins RecA, RecBC, RuvB, and RuvC, and induction of the SOS DNA damage response regulons, including error-prone DNA polymerase (poi) IV (DinB). Mutant cells with adaptive point mutations have been shown to arise from a subpopulation of hypermutable cells and to have a large number of unrelated mutations. Adaptive point mutation is a phenomenon that is currently undergoing active investigation by a number of laboratories in the United States and in other countries; these investigations are expected to discover additional characteristics and/or genes involved in the process.

The term “assay” is used along with the term “SR assay.” Both terms are intended to refer to the stress resolution assay, which is abbreviated as the SR assay. Any other assays will be referred to by the name that is used in literature reports or by the last name of the first author of the paper in which the assay is described.

Barrier function is the ability of some cell types, especially epithelial cells, to grow in a monolayer with the formation of cell-cell attachments. In some applications, barrier function can refer to the ability of these cells and their attachments to limit passage of substances.

Calibrated stress response-induction is a process that results in the induction of stress responses at a level that is calibrated or set by the operator. In general, four levels of cellular stress response induction can be induced: (1) very low level where some changes are induced or detected but changes in gene expression and/or stress response-related pathways are minimal or not observed, (2) low intermediate level where stress response pathways undergo expression changes, but persistent changes in cell morphology, behavior, and/or function are not observed, (3) high intermediate level where transient or permanent changes in cell morphology, behavior, and/or function are observed, (4) very high level were permanent changes in cell morphology, behavior, and/or function are observed. Calibrated stress response-induction conditions as used in the SR assay described herein can be optimized for any of these four levels of stress response, but currently the SR assay uses conditions optimized to induce expression of level #2, the low intermediate level, because this was found to be the optimal level for recovery of clones with mutations in the HPRT gene. Additional criteria for determining the calibrated stress conditions for a target cell type of interest for use in the SR assay described herein include: (1) the stress conditions should be high enough to induce expression of stress responses in cells, especially for investigation of conditions associated with the induction of genomic damage, DNA damage stress, and/or oxidative stress, (2) the conditions should be low so as to avoid induction of significant additional damage in cells as a result of the calibrated stress conditions, (3) the level should be optimized for recovery of cells with alterations in reporter genes, if that is a goal of the assay, and (4) the conditions should be such that criteria for an SRID-50% are met. One method for meeting criterion #2 is to identify calibrated stress conditions that can be resolved by parental, control, or vehicle-exposed cells without evidence of a significant increase in the cellular damage burden (defined below).

Calibrated stress response-induction condition(s) are the conditions that induce any one of the calibrated stress induction levels described above. These conditions vary by cell type and also appear to be affected by individual polymorphisms in both nuclear and mitochondrial DNA. These conditions may induce one or more cellular stress response levels at the same time.

Cell cycle checkpoint and/or checkpoint function are points or places in the cell cycle where cells can stop their progression through the cell cycle. The most common ones are the G(2)M and G(1)S arrests. Cells also can arrest in S phase, and there is some evidence that some cells may arrest in M phase as well. Cell cycle arrest is induced as a response to stress, and it provides an opportunity for cells to repair damage before re-entering the growth cycle. In the SR assay, cell cycle arrest is used as one measure of parental cell function; the inability to express a cell cycle arrest following exposure to stress conditions is used as one indicator to classify clones as Phen-alt clones (see below).

Cell death pathways are pathways or processes that remove cells from the cell cycle permanently. For the SR assay these processes are considered to include apoptosis, necrosis, senescence, and/or pathways that use combinations of the aforementioned pathways. A recently recognized exception to this definition is discussed below (see checkpoint adaptation). It is possible that other exceptions or variations on this definition will be recognized in the future.

Checkpoint adaptation (also called checkpoint abrogation, post-senescence emergence, or emergence from senescence) is a recently recognized process, believed to be expressed by epithelial cells but not fibroblasts, and mediated by oxidative stress pathways. This process allows cells with damage to re-enter the growth cycle and replicate in spite of the presence of damage and in spite of the fact that they have undergone senescence. In the SR assay, this damage tolerance mechanism and its associated pathways are postulated to be used by the Phen-alt clones to escape from senescence and re-enter the growth cycle. This phenomenon is postulated to be related to oxidative stress response mechanisms and to have important roles in the pathogenesis in a variety of human diseases. It currently is undergoing investigation by several laboratories and additional information about this phenomenon is expected to be reported in the future.

Chromatin is a macromolecular complex composed largely of the DNA and protein that make up chromosomes. It is found inside the nuclei of eukaryotic cells. In its condensed form it is referred to as heterochromatin; in its extended form it is referred to as euchromatin. Components of chromatin include DNA, histone proteins, non-histone proteins, histone variant proteins, RNA elements and accessory elements including but not limited to polyamines.

Cloning efficiency(ies) (CE(s)) are a calculated number used to quantify the frequency of occurrence of cells with characteristics of interest in a larger cell population. CEs can be used for determining the frequency of surviving cell clones in cell populations tested in the SR assay, CEs provide an estimate of the number of cells in the original population that had the characteristic of interest. In the SR assay, CEs can be calculated as part of the midway assessments (mCE) and again as part of the final assessments (fCE). The formulas used to calculate these CEs are different and thus cannot be compared directly. The formulas for calculating mCEs and fCEs are provided in the methods. The formulas for calculating CEs are based upon Poisson distribution theory, and thus, they only can be applied accurately to data obtained in assays that comply and conform to Poisson distribution theory and limitations, as does the SR assay.

Conditioned medium is cell culture medium that has been ‘conditioned’ by using it to feed cells in log phase growth for a short period prior to using it to feed another group of cells. The purpose behind the use of conditioned medium is to allow growth factors and other components produced by cells in log phase growth to enter the medium, and then to be delivered via transfer of the medium to a second set of cells. See the specific methods section for the methods involved in making this medium.

Damage avoidance pathway(s) are cellular stress response and/or resolution pathways, mechanism(s), or mode(s) of action that are expressed and/or utilized by cells and that result in, or are associated with, minimal and/or non-significant change/alterations in endpoints of interest. Damage avoidance pathways maintain or restore homeostasis, prevent or repair cellular damage, and maintain the integrity of the genome. The expression of these pathways results in avoidance of most but not all cellular damage. In the SR assay, damage avoidance pathways are judged to have been used by surviving cell clones to resolve the calibrated stress conditions for clones that retain parental cell morphology and critical functions, which are selected based upon the cell type used in the assay. For example, for epithelial cells the functions assessed are barrier function and cell cycle checkpoint arrest. The fCEs of Phen-parental clones are used as a quantitative indicator of the functionality of damage avoidance pathways. The CEs of Phen-alt-rev clones may be included in this assessment. For use with the SR assay, the terms ‘damage avoidance pathways’ also are intended to include pathways described in the literature as damage avoidance pathways.

Damage burden is the sum of all types of damage in a cell, without specifying the exact type of damage present.

Damage tolerance pathway(s) are cellular stress response and/or resolution pathways, mechanism(s), or mode(s) of action that are expressed and/or utilized by cells to resolve the calibrated stress conditions of the SR assay and that result in, or are associated with significant change/alterations in endpoints of interest. Expression or utilization of these pathways results in failure to maintain homeostasis, to prevent or repair cellular damage, and/or to provide protection to the integrity of the cellular genome. These pathways are associated with tolerance by the cell of damage and allow the cell to reenter and/or to progress through the cell cycle in the presence of damage. These pathways also can provide transient adaptation of stress conditions. When used in conjunction with the SR assay, the terms ‘damage tolerance pathway(s)’ or ‘damage accumulation pathway(s)’ or ‘damage tolerance/accumulation pathway(s)’ are interchangeable. In the SR assay, damage tolerance pathways are judged to have been expressed by surviving cell clones that have altered phenotypes (Phen-HPRT⁻ and Phen-alt clones, either reversible or irreversible), with loss of some or many parental cell characteristics, inability to express a cell cycle arrest upon exposure to stress conditions, and/or with evidence of damage in DNA. The CEs of Phen-alt and Phen-HPRT⁻ clones are combined as a quantitative indicator of the functionality of damage tolerance pathways. The CEs of Phen-alt-rev clones may be included in this assessment. For use with the SR assay, the terms ‘damage tolerance pathways’ also are intended to include pathways described in the literature as damage tolerance pathways.

Effect per unit dose is the effect per unit dose is calculated as the effect of interest divided by the amount of agent used in the experiment. Thus, the effect per unit dose of the calibrated stress conditions exposure upon CEs for Phen-parental clones from ENU-exposed cells is equal to 95÷35 micrograms 6-TG=2.7 CE/microgram 6-TG. (Molarity and/or cumulative dose of 6-TG or the agent used to induce the calibrated stress conditions also can be used).

Endpoint(s) of interest are assessment outcomes or data point(s) that are informative for the purpose of determining the effects of an exposure to an exogenous agent or condition, or for determining the ability of cells to resolve one or more exogenously induced stress response(s). Endpoints of interest also include any published or reported endpoints that are sued to evaluate cells used in the SR assay.

Epigenetic-based cellular memory and associated processes (epigenetic cell memory, ECM). This phenomenon and its processes have been hypothesized to exist as part of the phenomenon referred to as cellular memory, with roles in the regulation of global patterns of gene expression and protection of the genome by silencing viruses and transposons [Eilertsen et al. 2007]. Mechanisms involved in this type of memory include DNA methylation and histone modifications (see Table 1 for additional hypothesized systems). Histone modifications are hypothesized to be involved in transient cellular memory changes, while DNA methylation changes are hypothesized to be involved in persistent and/or irreversible changes. It is believed that the roles of these epigenetic changes in cell memory may have considerable overlap and a better understanding of their roles remains to be elucidated.

Epigenetics is the field of investigation focused upon the study of modifications of chromatin, other than the primary DNA sequence, plus associated protein factors, that have information content and that are preserved during cell division. The epigenome, or sum of genome-wide epigenetic patterns, differs between cells/cell types, thus allowing distinctions between tissues or organs, stein cells from somatic cells, and aged from young cells. The epigenome is influenced by sex, age, diet, stress responses or stress status of the cell or cell population, genotype and drug exposures. The cancer epigenome may be the best studied example of a disease epigenome.

Epigenetic code replication machinery is a complex hypothesized to be composed of enzymes involved in DNA methylation and histone modifications such as DNMTs (DNA methyltransferases), HATs (historic acetyltransferases), HDACs (historic deacetylases including siruins) [Bronner et al. 2007], A range of cell systems, processes, and elements beyond those described above, have been hypothesized to have roles in the maintenance and restoration of ECM.

Epigenome is that part of chromatin that exists “above” or “in addition to” the base sequence code. It is referred to by some investigators as ‘the epigenetic landscape’ or epigenetic profile. It currently is considered to consist of two units; the first is part of the covalent structure of DNA and consists of the methylated cytosines located in the dinucleotide sequence CG (other methylated bases also may make up part of this unit of the epigenome). The second unit consists of a non-covalent component composed of histone proteins, non histone proteins, various forms of RNA, and additional accessory elements such as but not limited to polyamines. Some investigators consider the epigenome to consist primarily of the chemical modifications that are made to the elements that surround the DNA bases. Another definition includes the DNA bases that are not transcribed into mRNA and that make up about 90% of the human genome. For some authors, the epigenome also is considered to include nuclear components that affect gene expression. Because knowledge of this area is increasing rapidly, it is probable that the definition of ‘epigenome’ will be altered or adjusted in the future to accommodate new information. In the SR assay, the term epigenome is used to refer to the nuclear epigenome and/or the mitochondrial, epigenome.

Epigenetic alterations or modifications are changes, either induced or spontaneously occurring, in the epigenome. Currently, these changes are recognized to consist of changes in histones incorporated into chromatin (including but not limited to the portions of incorporated histone variants), post-translational modifications (including but not limited to methylation, acetylation, phosphorylation, formylation), changes in the proportions and types of nucleic acids associated with chromatin (including but not limited to RNA elements), and the proportions and amounts of other elements or factors associated with chromatin (including but not limited to polyamines). Processes that affect the epigenome include but are not limited to DNA methylation, histone modifications, nucleosome repositioning, higher order chromatin remodeling, non-coding RNA patterns, and RNA and DNA editing [Mehler 2008]. Because knowledge of this area is increasing rapidly, it is probable that the understanding of epigenomic alterations will be expanded in the future to accommodate new information.

Gene bookmarking is a process defined as “the process of remembering patterns of active gene expression during mitosis for transmission to daughter cells” [Sarge and Park-Sarge 2009]. Chemical marks involved in gene bookmarking include histone post-translational modifications, DNA methylation at CpG islands, and small nuclear RNAs processes. It is believed that gene bookmarking is achieved through control of the degree of compaction of promotor regions of specific genes; for example, heat shock factor 2 protein (HSF2) binds promotor elements of heat shock genes, thereby bookmarking the region [Wilkerson et al. 2007, Murphy et al. 2008]. Gene bookmarking is a recently recognized phenomenon and it is probable that additional information about gene bookmarking mechanisms will be recognized in the future.

Genome or genotype is the specific sequence of nucleoside bases that make up DNA. In the SR assay, the genome and/or DNA refer to nuclear DNA and/or mitochondrial DNA.

Genome stability is a cellular state and/or mechanism postulated to be associated with or responsible for the maintenance of the integrity of the genome. Considered to be the converse of genomic instability.

Genomic instability is a cellular state and associated processes or mechanisms that fail to maintain genome integrity. The state of cellular genomic instability may be transient or permanent. Evidence for this state comes from the findings of mutations in DNA, phenotypic alterations in cells, and/or evidence of loss of ECM as evidenced by loss of parental cell phenotype and changes or loss in cell behavior and/function. Some investigators believe that genomic instability can affect the entire genome; other use this term to refer to loss of integrity in DNA and use the term ‘epigenomic instability’ to refer to a similar phenomenon in the epigenome.

HAT-medium is medium composed of hypoxanthine, cytidine, thymidine and aminopterin is abbreviated HAT⁻ medium. This medium is used to remove HPRT⁻ cells from a cell population without inducing cell death in wild-type cells, or to test cells for expression of the HPRT⁻ phenotype, (See the specific methods section of methods used to make this medium).

Latency, as used in the SR assay, is the time period from the end of the calibrated stress conditions exposure steps of the SR assay and the appearance of viable cells clones.

Mechanism: the terms mechanism(s), mode(s) of action, and pathway(s) are used interchangeably in the discussion of the SR assay.

Phen-alt clones are phenotypically altered clones as detected using the SR assay. These clones have morphologic alterations, do not form cell-cell attachments, do not grow in a monolayer similar to that of parental cells, and/or do not express a cell cycle arrest upon re-exposure to the calibrated stress conditions. These changes may be reversible or irreversible. These cell clones are resistant to 6-TG and some have a tumorigenic phenotype.

Phen-HPRT⁻ clones are phenotypically HPRT gene-deficient clones detected using the SR assay. These clones are similar to parental cells but they are resistant to 6-TG due deficiency of the HPRT gene.

Phen-parental clones are phenotypically parental cell-like clones detected using the SR assay. These clones retain parental cell functions, including barrier function and cell cycle arrest function, and also retain parental cell morphology.

Poisson distribution. In probability theory and statistics, the Poisson distribution is a discrete probability distribution that expresses the probability of a number of events occurring in a fixed period of time if these events occur with a known average rate and independently of the time since the last event. The Poisson distribution also can be used for the number of events in other specified intervals such as distance, area or volume.

Reporter gene is any gene that can be studied using gene locus-specific mutation assays and used as an indicator of alterations introduced into the genome of target cells.

Selection/selection process(es) are process(es) by which cells with one phenotype of interest are separated from other cells with a differing phenotype. Most currently available gene locus-specific genomic damage assays rely upon the use of a step in the methods that is referred to as the ‘selection step’.

Selection conditions are conditions used to achieve selection; these are conditions that can be applied to cells/cell populations for the purpose of separating cells by phenotype as described above. The criterion for this process is that the selection conditions/process should not induce additional changes in the cells/cell population to which they are applied. Note that selection conditions differ from calibrated stress conditions because the latter are not based upon the assumption that no additional changes in the cell population are introduced. In contrast, calibrated stress conditions have the goal of inducing changes in stress response expression so that the outcomes of those changes can be evaluated.

SRID-x % is the stress response inducing dose-50%. This term refers to the level of agent used to induce the calibrated stress conditions of the SR assay. The x % refers to the ratio of [(total negative wells/total wells)×100%)] of a 96 well tissue culture vessel, or other appropriate tissue culture vessel, converted to a percentage. Due to Poisson distribution considerations, the optimal ratio is 50%. When used in the SR assay, the level of agent used to induce the calibrated stress conditions is affected by plating density of the cells, by cell type, and by the duration of the exposure, so these should be included in defining a SRID-x %. For example, for CDNR4 cells and 6-TG used as the stress inducing agent, the SRID-50% was determined empirically to be 35 micrograms/ml 6-TG when applied to cells plated at a density of 500 to 600 cells per well of a 96 well tissue culture plate and exposed for four days.

Stress is any condition in which the cell or cell population cannot continue to carry out functions associated with homeostasis (including but not limited to survival), and/or that elicits a change in gene expression, and/or a change in cell function, either transient or persistent, in the cell or cell population. (Also see stress response-induction condition(s) below).

Stress-inducing agent or stress environment is an agent or condition that induces one or more types of stress responses in cells. Agents or conditions are considered to be stress inducers or stress environments if they induce such responses at some level or duration of exposure, even if the level or duration of exposure in a particular experiment is not sufficient to induce a detectable change in cells.

Stress-associated mutagenesis is mutagenesis associated with expression of both the damage tolerance-reversible and damage tolerance-irreversible groups of pathways; this type of mutagenesis has been recognized for several decades and is referred to as stress-induced mutagenesis, untargeted (non-targeted) mutagenesis, or environmental mutagenesis. The terms stationary phase mutagenesis, hypermutation, genomic instability, and imitator phenotype also are applied to subpathways and/or to manifestations of these damage tolerance resolution pathways in cell/cell populations.

Stress response pathway(s) are pathways or mechanisms, including the associated genes and molecular elements that undergo a change in expression in cells that have been exposed to a stress inducing agent. These pathways are expressed by cells following exposure to an environmental change, a stress inducing agent or condition and are used as described above to respond to and to resolve stress conditions.

Stress response-induction determination is any endpoint that currently is known to be informative for stress response induction can be used. Examples (not inclusive) include evidence of adduct formation in proteins or nucleic acids, changes in gene expression, changes in mRNA levels, changes in protein levels, changes in oxidation products, changes in lipid profiles, changes in reactive oxygen species production or levels, or cell cycle checkpoint arrest.

Stress response-induction condition(s) are any condition(s) that induce the expression of stress responses in cells. These conditions can be extracellular, intercellular (between cells in a cell population), or intracellular (such as mitochondrial stress or endoplasmic reticulum stress), which then is communicated throughout the cell, and usually, to the surrounding cell population. Stress response-induction conditions (calibrated stress conditions) do not meet the above criterion for successful selection and they do not claim to do so. This represents the significant difference between the process(es) of selection and the process(es) of inducing a stress response or a calibrated stress response (see Table 5).

Stationary phase is one stage of the cell cycle in which no change in population size is displayed.

Stationary phase mutagenesis is a type of mutagenesis that is hypothesized to occur in microbes, animals and humans in cells that are stalled in stationary phase as a result of a stress environment.

Stress-associated mutagenesis is expression of damage tolerance pathways with resultant induction of mutations in DNA is referred to by several alternative names including stress-induced mutagenesis, adaptive mutagenesis, and an early name was environmental mutagenesis. In addition, the term untargeted (or nontargeted) mutagenesis is used to refer to these pathways by some investigators, while these terms are used by others to refer to the damage tolerance pathways associated with genomic instability, thus, resulting in ambiguity in the literature. Damage tolerance stress response resolution pathways are referred to as either damage tolerance-reversible (damage tol-rev paths) or damage tolerance irreversible (damage tol-irr paths); potential differences between these pathways are presented in the background material.

Targeted mutagenesis is a mutagenic process that results in genetic alterations in nuclear DNA and/or mitochondrial DNA following exposure to endogenous or exogenous DNA damaging agents. In this type of mutagenesis, mutations are formed at the site of induced lesions.

Test agent or condition is a chemical, drug, physical agent, condition, or other substance or condition for which there is a desire or need to determine its effects, including but not limited to toxic or beneficial effects.

Untargeted mutagenesis is a mutagenic process(es) resulting in the induction of changes in cellular DNA at sites distant from the site of an induced lesion. Untargeted imitation is a phenomenon reported in several different microbiological systems and that can occur via several different molecular mechanisms [Hersh et al., 2004]. Untargeted mutagenesis is a phenomenon that currently is undergoing active investigation by a number of laboratories in the United States and in other countries; these investigations will discover additional characteristics and/or genes involved in the process, identification of additional characteristics and/or genetic pathways regulating this response are expected to be identified in the future.

Abbreviations are used herein as follows:

6-TG (6TG): the chemotherapeutic agent six-thioguanine (6-thioguanine).

AKT: protein kinase B.

ATM: ataxia telangiectasia mutated.

ATR: ATM/Rad3-related protein.

CE(s): cloning efficiencies. See the methods for the formula used to calculate this value in the methods.

fCE: final CE. Used to refer the CEs calculated as part of the SR assay final assessments. The formula for calculating fCEs is different from that used to calculate mCEs.

mCE: midway CE. Used to refer to CEs calculated as part of the SR assay midway assessments. The formula for calculating mCEs is different from that used to calculate fCEs.

Chk1: checkpoint protein 1.

Chk2: checkpoint protein 2.

ENU: ethylnitrosourea.

Damage tol-rev: damage tolerance-reversible pathways; one of several groups of cellular stress response resolution pathway outcomes.

Damage tol-irr: damage tolerance-irreversible pathways; one of several groups of cellular stress response resolution pathways. There may be considerable overlap between damage tol-rev and damage tol-irr pathways.

HPRT: the hypoxanthine phosphoribosyl transferase gene is abbreviated HPRT (human) or Hprt (animal). The protein is abbreviated hprt. The HPRT gene is classified among the housekeeping genes and is located on the X-chromosome of the cells of many human and rodent cells. It is expressed in all cells, and is involved in salvaging guanine as a component of the purine-salvage pathway. Cells with a wild type (functional) HPRT gene are referred to as HPRT⁺ cells; cells with a mutation in the HPRT gene that renders it non-functional are referred to as HPRT⁻ cells. Cells that are HPRT⁻ cannot salvage guanine and therefore must use an alternative pathway known as the de novo purine/pyrimidine biosynthesis pathway to produce guanine for use in DNA metabolism and replication. HPRT also is recognized as a reporter gene used in some gene locus-specific assays.

In: natural log.

MNNG: N-methyl-N′-nitro-N-nitrosoguanidine.

MNU: methylnitrosourea.

PIG-A: phosphatidylinositol glycan type A; one of seven genes that code for the proteins constituting glucosamine acetyl (GlcNAc) transferase, which is a multimeric enzyme that mediates the first step in glycosylphosphatidylinositol (GPI) anchor biosynthesis. The seven genes involved in this biosynthetic first step are PIGA, PIGH, PIHC, GPI1, PIGP, DPM2, and PIGL (other names for these genes also may be in use). The PIG-A gene currently is being investigated as a potential reporter gene.

Phen or Ph: phenotype.

PNH: paroxysmal nocturnal hemoglobinuria.

REV3: the name given to the gene that encodes the catalytic subunit of DNA polymerase zeta. The term Rev3 refers to the protein.

SR: stress response(s) or stress resolution; the collection of responses expressed by a cell or cell population as part of the process of recognizing, signaling, resolving or adapting to a stress environment.

SRID-x % (the stress response induction dose-x %): the level of stress inducing agent that reduces cells survival to 1 or fewer clones per well in x % of the wells of a 96 well tissue culture plate (or other locus discrete tissue culture vessel) when the cells are plated as a low density (see methods for how to calculate x). For example, the SRID-10% for cell line Y is the level of agent used in the calibrated stress conditions step of the assay that reduces cell survival to 1 or fewer clones per well in approximately 10% of the wells of a 96 well plate when the cells are plated at a low plating density.

TLS: translesion synthesis pathway

XPA: xeroderma pigmentosum A protein.

### EXAMPLES

The subject matter of this disclosure is now described with reference to the following Examples. These Examples are provided for the purpose of illustration only, and the subject matter is not limited to these Examples, but rather encompasses all variations which are evident as a result of the teaching provided herein.

### Examples of SR Assay Application

Example #1 describes the use of the entire assay to determine all assay endpoints associated with resolution of a DNA damage stress response. The remaining examples describe use of the SR assay for more limited purposes to assess one or more assay subcomponents or endpoints.

### Example 1

Quantification of stress response resolution pathway expression/functionality following exposure to vehicle alone, or ENU. Quantification of damage avoidance versus damage tolerance stress response resolution pathway expression and functionality in the above cells, comparison of final surviving cell population damage burden.

(Note: this example demonstrates use of the entire assay with all of its endpoints to evaluate the effect of pre-exposure of CDNR4 cells to the mutagen ENU).

The murine mammary epithelial cells designated CDNR4 were grown in their preferred growth medium (42% DMEM, 42% Hams F-12, 12% FBS, 1% 100×L-glutamine, 1% pen-strep, 0.5% to 1% bovine pituitary extract) until approximately 6 million cells were available. Cells were removed from the expansion flasks, counted, and replated at a rate of 500,000 cells per 75 mm flask in growth medium. The flasks were assigned randomly to one of two groups: a vehicle-alone exposure (control group); an ENU-exposure (treated) group. For flasks of ENU-exposed cells, ENT was added to the growth medium to achieve a final concentration of 20 micrograms per milliliter. For group 1 flasks, a volume of growth medium equal to the volume of ENU was added to the flasks. The cells were exposed to this solution for 1 hour at 37° C.; at the end of this time the solution was removed, the cells were washed with PBS at 37° C., and the cells were re-fed with fresh growth medium.

Within 24 hours after the exposure, ENU-exposed cells showed some signs of stress as evidenced by a drop in growth medium pH resulting in a visible yellow color to the medium. This drop in pH recurred within six to 12 hours after re-feeding the cells with fresh growth medium. To reduce this problem, all cells (vehicle-exposed and ENU-exposed) were re-fed every day with medium that was buffered with HEPES at a concentration of 25 millimolar. This reduced the pH changes in the medium, but did not resolve the problem completely. During this time period a slight non-significant increase in cell death among cells in ENU-exposed flasks but not vehicle-exposed flasks was observed. This process of increased cell death among ENU-exposed cells resolved after approximately 10 to 12 days. Morphologic changes also were visible in flasks of cells exposed to ENU compared to control flasks, but these changes also resolved within 10 to 12 days. The cells were maintained on growth medium for 2 weeks and re-fed every 3 days or more often as necessitated by color changes in the growth medium.

Midway Assessments:

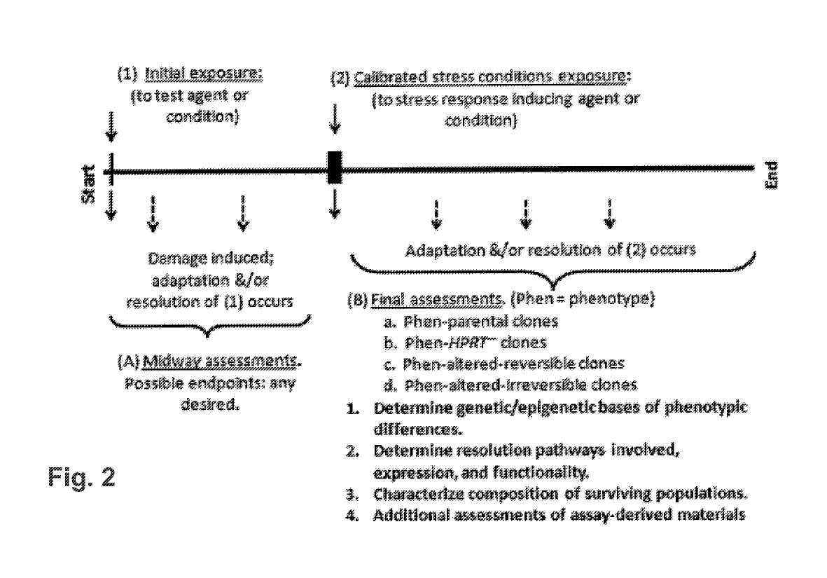

Two weeks after exposure of treated cells to ENU, no differences in the criteria listed in Table 6 were observed between vehicle- or ENU-exposed cells, mCEs plates were prepared and evaluated as described above (see method ‘B’) and the average mCEs for control and treated cells were determined to be 33.4% and 34.2%, respectively (see FIG. 2A). There was no significant difference in mCEs, as a measure of pre-calibrated stress conditions pro-survival pathway function, in vehicle-exposed versus ENU-exposed cells. All cells, had similar morphology, growth rates and characteristics, and expressed a cell cycle checkpoint response following exposure to a stress-inducing agent (6-TG was used in this example). These assessments supported the conclusion that there were not significant differences in the epigenetic landscape of vehicle-exposed cells versus ENU-exposed cells. Average SRID-50% values for both vehicle and ENU-exposed cells plated at the rate of 600 cells per well were 35 micrograms/ml and 2.5 micrograms/ml, respectively (p<0.05).

Calibrated Stress Conditions Exposure:

At the end of 2 weeks, the cells were removed from the flasks with trypsin/EDTA and passed through a 20 to 25 micron pore size sterile Nytran filter. The cell suspension was evaluated by light microscopy for evidence of large clumps, and was re-filtered if necessary using a new sterile Nytran filter of the same size to remove any apparent large clumps of cells. The cells were counted, re-suspended in growth medium with 30 micrograms/ml 6TG and plated on 96 well tissue culture plates at a density of 600 cells per well. The plates were incubated at 37° C. One to 3 days after the start of the SR induction phase of the SR assay, cell cycle arrest of all cells and death of greater than 90% of the cells was apparent. Cell death appeared to occur more rapidly and to be more complete on plates of treated cells compared to control cells. At the end of 4 days of selection, all plates were washed with PBS and re-fed with fresh growth medium or conditioned medium. Two to 3 days after the end of SR induction phase, cell death appeared to be close to 100% on all plates. A background of morphologically disordered, variably shaped (predominantly spindle-shaped) cells remained adherent to the tissue culture dishes, but these cells showed no evidence of growth and appeared to be dead, dying or senescent. Interestingly, the wells of plates containing EMU-treated cells appeared to have fewer cells (resulting in a ‘cleaner’ appearance) than comparable wells on control plates.

Final Assessments:

Evaluation of plates of ENU-exposed and vehicle-exposed cells 10 to 14 days post-selection revealed the presence of scattered small colonies of cells in clusters ranging from a few cells to up to 20 or more cells. During the same time period, variably sized colonies of viable, proliferating cells appeared in many wells of plates of control cells. Interestingly, no small colonies of cell clusters similar to those observed on plates of ENU-exposed cells were observed on any plates of vehicle-exposed cells, although it was recognized that the rapid appearance of viable colonies on plates of vehicle-exposed cells made it more difficult to identify colonies of cells with these characteristics. These small colonies of cells had parental cell morphology including monolayer growth, formation of intercellular junctions, low retractility, and other characteristics. All wells bearing such clusters were marked. Continued observation of the small cell clusters demonstrated viability, albeit with slow growth for the majority of these clones. Many of these clones were subsequently demonstrated to have mutations in the Hprt gene.

Approximately two weeks post-selection, visible, expanding colonies of cells could be identified on all plates. These were most numerous among vehicle-exposed cells. Similar colonies continued to emerge for 5 to 6 weeks post-calibrated stress conditions exposure, and the time for completion of this process was of much greater duration for treated cells than for control cells. By 7 weeks, no additional clones appeared.

All presumptive Phen-Hprt⁻ clones and at least 10% of the surviving clones on all of the plates were collected for phenotypic testing using the evaluation system presented in Table 7. Phen-Hprt⁻ clones were identified by their slow growth and poor viability in unsupplemented growth medium, by their substantially improved growth on medium supplemented with guanosine or folic acid, by their continued resistance to 6-TG, and by their sensitivity to killing by HAT− medium.

Phen-parental clones were identified by the presence of several characteristics and by phenotypic testing. Their morphology and growth characteristics were determined to be those of parental or wild-type cells using the criteria listed in Table 6. Phenotypic testing revealed that these clones grew well in growth medium, did not require medium supplemented with quanosine or folic acid, expressed cell cycle arrest when re-exposed to a stress environment such as that induced by 6TG, and were viable in HAT− medium. All clones isolated from control plates were shown to have this phenotype while only a small fraction of the clones isolated from treated plates were demonstrated to have this phenotype.

The majority of clones removed from plates of treated cells demonstrated an altered phenotype and were designated phenotypically-altered (Phen-alt) clones. These clones grew well in both unsupplemented growth medium and guanosine or folic acid supplemented medium, grew as well or better than parental cells in HAT− medium, and showed continued resistance to the cytotoxicity of 6-TG as evidenced by failure to express cell cycle arrest and cell death upon re-exposure. Cell clone resistance to re-exposure to 6-TG was variable and ranged from 30% to close to 100% resistance, depending upon the particular clone evaluated. The majority of clones showed between 30% to 60% resistance to 6-TG.

All clones showing at least 30% resistance were expanded further on growth plates, and were evaluated further. These clones were exposed 1 to 4 additional times to 6-TG at the calibrated stress conditions levels, followed by a several day period of recovery. Following the subsequent exposures, most clones showed increased resistance to the cytotoxic effects of 6-TG that reached a level approximating 100% resistance after 3 or 4 additional exposures to 6-TG. Once this level was reached, these clones were observed to fail to revert to a 6-TG sensitive phenotype. These clones were classified as Phen-alt-irr. A few clones reverted hack to a 6-TG-sensitive phenotype after a several week period during which they were re-exposed to 6-TG 1 to 4 times; these clones underwent an unexpected cell cycle arrest followed by large scale cell death upon exposure to 6-TG. The large scale cell death resulted in all cells of these particular clones being lost. These latter clones were classified as Phen-alt-rev.

Pro-survival pathway function for total, damage avoidance, and damage tolerance/accumulation pathways: The goal of these assessments was to use the fCEs for the phenotype-specific subgroups as indicators of pro-survival pathway function. Total pro-survival pathway function was determined as follows. The fCEs for all surviving cell clones among vehicle-exposed cells was 3400±120×10⁻⁶, while for ENU-exposed cells, it was 1210±380×10⁻⁶ (p<0.01). This represents an approximate 3-fold reduction in the total pro-survival pathway function of ENU-exposed versus vehicle-exposed CDNR4 cells. Damage avoidance pathway function was assessed as follows. In the SR assay, the fCEs of Phen-parental clones are defined as quantitative indicators of the functional capacity of damage avoidance pathways. Considering only Phen-parental clones, the reduction in damage avoidance pathway function between control and treated cells was approximately 25-fold (p<0.001 (T-test)). However, Phen-alt-rev also may be indicative of damage avoidance pathway capacity since these clones resisted the accumulation of additional damage during repeated exposures to the stress conditions and returned to a parental-like phenotype after several weeks. If Phen-parental and Phen-alt-rev clones for ENU-exposed cells are considered together, then the combined average fCE is 280±150×10⁻⁶. Compared to the average fCE for Phen-parental clones for control cells, this represents a 12-fold reduction in damage avoidance pathway function. Thus, the initial exposure to ENU reduced the expression and/or functionality of damage avoidance pathways in CDNR4 cells during subsequent stress conditions by between 12- to 25-fold. Damage tolerance pathway function was determined as follows. The findings of alterations in DNA (Phen-Hprt⁻ clones) and/or in phenotype (Phen-alt-irr clones) serve as quantitative indicators of the expression and/or functionality of damage tolerance pro-survival pathways that resulted in permanent alterations in the cellular genome. The fCEs for these clones among vehicle-exposed cells were estimated at 0.6×10⁻⁶, while the average fCE for Phen-Hprt⁻ clones and Phen-alt clones together for ENU-exposed cells was 870±220×10⁻⁶. If Phen-alt-rev clones are included, based upon the fact that they did not resolve the stress conditions with the same efficiency as the Phen-parental clones, the average CE for Phen-Hprt- and Phen-alt clones was 1100±350×10⁻⁶. These comparisons show that, while damage avoidance pathway outcomes were reduced among ENU-exposed cells, damage tolerance/accumulation pathway outcomes increased by an estimated 3000- to 3700-fold.

Phen-alt-irr clone fCES were considered to be indicative of loss of ECM and induction of a period of transient or persistent genomic instability, based upon reasoning described above. Thus, the fCEs of Phen-alt-irr clones served as an indicator or biomarker of the frequency with which permanent damage to ECM. The ability of the assay to detect damage to a cell maintenance system, as opposed to damage to specific sites on DNA or in the epigenome, is of particular importance because a number of investigators have postulated recently that damage to ECM maintenance and restoration systems is necessary for neoplastic transformation. As a result, the SR assay quantities the occurrence of events hypothesized to be directly related to cancer induction.

Composition of the post-stress resolution surviving cell population assessments were assessed as follows. Using the estimates for the occurrence of Phen-HPRT⁻ clones and Phen-alt-irr clones among the surviving control cell population, 0.00006% (average 0.6 out of 10⁶ cells) of the control cell population was estimated to be composed of cells with irreversible damage. Thus, for these endpoints, the surviving control cells were not significantly altered from the parental cell population.

Phen-parental clones made up only 9% (average 3 out of 38 clones) of the final surviving treated cell population. In contrast, 15% (average 6 out of 38 clones) of the surviving cell clones had evidence of DNA damage in the Hprt gene, 17% (average 6 out of 38 clones) had evidence of reversible phenotypic alterations, and 60% (average 23 out of 38 clones) had evidence of irreversible phenotypic changes with loss of epithelial cell functions (i.e. the Phen-alt-irr clones). Using mutations in DNA and irreversible phenotypic alterations as indicative of permanent alterations in the genome, these figures show that 75% of the surviving ENU-exposed population had permanent genomic changes, if the Phen-alt-rev clones are included in this latter group as indicative of cell clones with damage, then 92% of the surviving ENU exposed cell population consisted of cells damage that was sufficiently severe to alter morphology and phenotype.

The above experiments were repeated multiple times, and some experiments used MNU as the pre-exposure agent in place of ENU. The results of all experiments were similar, and the results of the experiments with ENU are presented here as representative.

### Example 2

Determination of the SRID-50% level for CDNR4 cells and quantification of post-calibrated stress conditions exposure pro-survival pathway functionality.

For these experiments, CDNR4 cells, which are considered to be p53 incompetent due to an estimated p53 capacity of <10% of normal, were used. As part of a pilot study, the mCEs of the CDNR4 cells was determined using method ‘B’ described above. The average in GE for three plates was determined to be 33.4%.

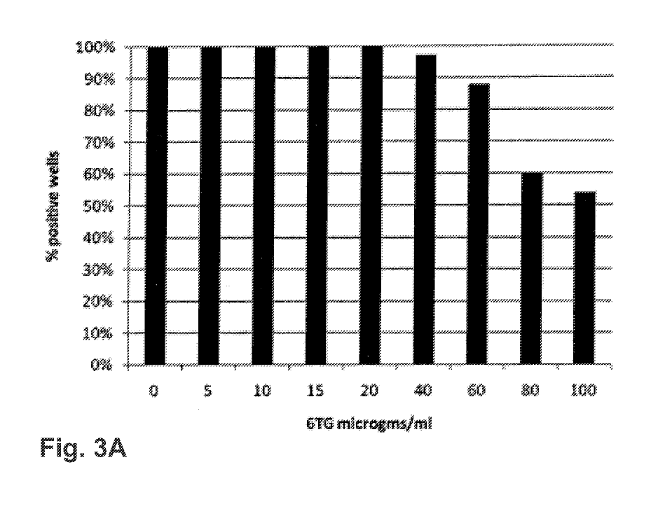

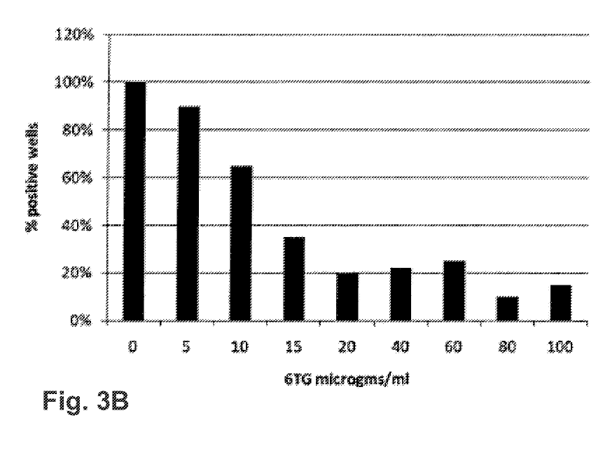

To determine the SKID-50% for these cells, the cells were grown in their preferred growth medium (42% DMEM, 42% Hams F-12, 12% FBS, 1% 1.00×L-glutamine, 1% 1.00× Pen-Strep, 0.5 to 1% Bovine Pituitary Extract) until approximately 6 million cells were available. Cells were removed from the expansion flasks, counted, and replated at a rate of 100,000 cells per 100 mm tissue culture dish in growth medium that contained variable amounts of 6TG ranging from 1 microgram/ml to 100 micrograms/ml. As an alternative, cells were plated at a rate of 600 cells per well on 96 well tissue culture plates. These plates were exposed to the same range of 6TG concentrations as were used for the 100 mm dishes. In this example, 6TG was used as the stress inducing agent to induce a DNA damage response. Other stress responses, such as a cytotoxic stress response, also may have been induced. All cells were observed to undergo cell cycle arrest within 24 hours after the start of the plating and 6-TG exposure period. Four days later, the cells were re-fed with conditioned growth medium that lacked 6TG. Over the next week, individual clones of surviving cells that had re-entered the growth cycle were identified on all plates. At 10 to 14 days after the start of the stress induction step of the assay (the start of the 6-TG exposures), three plates exposed to each dose of 6-TG were evaluated to determine the number of surviving clones per plate. Dose response curves for total surviving cell clones were generated for the data. The results of this study are presented in FIGS. 3A and 3B. This figure shows that the SRID-50% for vehicle-exposed CDNR4 cells was 35 micrograms/ml 6-TG but for ENU-exposed cells it was reduced to 2.5 micrograms/ml 6TG.

### Example 3

Quantification of stress response resolution pathway expression and/or functionality following exposure to vehicle alone, or ENU, using the SR assay and calibrated stress conditions with a SRID-50% instead of a SRID-10%.

The SR assay was conducted as above with the exception that the calibrated stress conditions level used in the assay was that determined to be equivalent to the SRID-70% to 80% instead of a SRID-50%. This higher level of stress response induction resulted in significant differences in assay outcome.

In this version of the assay, the cells of interest (CDNR4 cells) were exposed to a high calibrated stress conditions level to determine effects of a pre-exposure to an agent or condition of interest, upon the expression and functionality of stress response resolution pathways that are expressed under conditions of high level and/or prolonged stress. In this example, the agent used was ENU. The total survival frequency for control cells was approximately 700×10⁻⁶, and the total survival frequency for treated cells was approximately 128×10⁻⁶. When the surviving clones were sub-classified as to type, no Phen-HPRT⁻ clones were found among treated cells, and the majority of surviving treated cell clones had a phenotype consistent with that of Phen-alt-irr clones. This experiment was repeated several times with the goal of attempting to identify and isolate Phen-HPRT⁻ clones, without success. These efforts resulted in changes to the SR assay so that the recovery of Phen-HPRT⁻ clones was optimized. This led to the discovery that these particular clones were recovered most easily using a SR induction level in the range of the SRID-10% to 50%.

A few studies have been performed to evaluate stress response induction levels that were even higher than the SRID-70% to 80%; these experiments found that the survival of treated cells increased, rather than decreased, as the stress response induction level increased. This phenomenon may represent an example of checkpoint adaptation (Martien and Abbadie 2002). This phenomenon appears to reflect predominantly cellular responses to the stress environment and not to be informative for the effects of the pre-exposure; thus, experiments using these high levels of calibrated stress conditions were not pursued further and should be used with caution when evaluating the affects of exogenous agents upon stress resolution pathways.

### Example 4

Comparison of total post-calibrated stress conditions exposure pro-survival pathway(s) functionality for two human cell lines of the same epithelial cell type but derived from two different individuals. Both individual had long cigarette smoking histories (40 pack years each).

Two immortalized, human bronchial epithelial cells lines were expanded in their preferred growth medium and plated at the rate of 600 to 1000 cells per well in tissue culture wells of 96 well plates. Both cell lines were immortalized using hTERT technology. The cells were exposed to variable 6-TG concentrations that ranged up to 150 micrograms/ml 6-TG. For cell line #1; the SRID-10% dose of 6-TG was determined to be 110 micrograms/ml; for cell line #2 the SRID-10% dose of 6-TG was determined to be 60 micrograms/ml 6-TG. The mCE of cell line #1 was 41%; for cell line #2 it was 25%. Thus, the total pro-survival pathway functionality for cell line #1 was 49.2 (0.41×110 microgms/ml 6-TG); for cell line #2 this value was calculated to be 15.

Cell line #1 had an approximate 3.3-fold higher total pro-survival capacity than that of cell line #2. These findings are of interest because the person from whom cell line #1 was derived had a 40-pack year history of smoking but did not develop lung cancer, while cell line #2, was derived from a matched individual with the same smoking history but who did develop lung cancer. These findings support the conclusion that a direct relationship exists between damage avoidance pro-survival pathway functionality and resistance to cancer induction.

Note that in this example there was no initial exposure step that was conducted as part of the assay. Here the assay was used to take advantage of exposures that had occurred in vivo, in human beings, and in the past. Application of the SR assay was used to assess the effects of past cigarette smoke exposures upon stress response outcomes in two different individuals. The results showed that the outcomes of the cigarette smoke exposures were significantly different between the two people.

### Example 5

Quantification of pre- and post-calibrated stress conditions exposure total (damage avoidance and damage tolerance) survival frequencies for CDNR4 cells exposed to vehicle or to ENU.

CDNR4 cells were grown in their preferred growth medium and exposed to vehicle or ENU as described above (see example #1).

On day 14 post-ENU exposure, cell survival as determined by mCEs. There were no significant differences in pro-survival pathway function between the two groups of cells (mCE for vehicle-exposed cells was 33.7%; for ENU-exposed cells it was 34.2%). These mCE values provide information about the functionality of cellular survival pathways that function under conditions of mild stress associated with in vitro cell culture, trypsinization, processing, and exposure to fresh tissue culture medium. These pathways may be distinct from those expressed during stress response resolution.

Next, a stress response was induced using the SRID-×10% of 6-TG as determined for CDNR4 cells, and post-calibrated stress condition fCEs were quantified using SR assay techniques. Vehicle-exposed cells had fCEs of 3,400×10⁻⁶, while comparable values for ENU exposed cells were 95×10⁻⁶ (see FIGS. 3A and B, respectively). These results showed that cellular pro-survival pathways involved in successful stress response resolution had been altered significantly by the ENU exposure. These experiments also showed that the SR assay is capable of detecting changes in cellular stress response pathway functionality that cannot be recognized in the absence of an exogenously-induced stress response. Direct comparisons between pre- and post-calibrated stress conditions exposure CEs cannot be made because these values are determined using difference formulas.

### Example 6

### Use of the SR Assay to Determine the Frequency of One or More Sub-Classes of Surviving Clones; Example Using Phen-HPRT⁻ Clones in Epithelial Cells

CDNR4 cells were grown and exposed to vehicle alone or to ENU as described above. Within the first 2 weeks post-calibrated stress conditions exposure all plates were evaluated for the presence of scattered small colonies of cells in clusters ranging from a few cells to up to 20 or more cells. During the same time period, variably sized colonies of viable, proliferating cells appeared in many wells of plates of control cells. Interestingly, no small colonies of cell clusters similar to those observed on plates of ENU-exposed cells were observed on any plates of vehicle-exposed cells. It was recognized that the rapid appearance of viable colonies on plates of vehicle-exposed cells made it more difficult to identify clones with these characteristics. These small colonies of cells had parental cell morphology including monolayer growth, formation of intercellular junctions, low retractility, and other characteristics. All wells bearing such clusters were marked. Continued observation of the small cell clusters demonstrated viability, albeit with slow growth for the majority of these clones.

When the above identified clones had expanded to sufficient size to allow them to be subcloned (the typical size was a clone that occupied 30 to 50% or more of a single well of a 96-well tissue culture dish), the clones were transferred and subdivided for the purpose of phenotypic testing. All clones that had the characteristics of Phen-HPRT⁻ clones as shown in Table 7 were designated as such a representative subfraction was sequenced to confirm the presence of DNA base changes.

Remaining clones were discarded without further analysis. This limited application of the assay has the advantage that it can be completed more rapidly than the full assay, and it provides information about mutagenesis at the HPRT locus in epithelial cells that can be used for direct comparison to results in other cell types.

### Example 7

### Use of the SR Assay to Determine the Frequency of PIG-A-Deficient Clones Before and After Induction of a Stress Response

Cells of interest are expanded in their optimal growth medium and exposed to an agent or condition of interest as described above, or as appropriate for the particular cell line and the goals of the experiment. Subsequent to this exposure, cells are removed from the tissue culture vessel and prepared for flow cytometric analysis of GPI-anchored proteins or GPI anchors themselves, in accordance with the specific methods described above. Measurement of PIG-A deficient cells serves as an indicator of the damage burden of the cell population and is particularly useful since alterations at this locus can be determined without the need to induce a stress response as part of the assessment of mutagenesis. PIG-A deficient cells can be assessed before and after the calibrated stress conditions exposure for comparison of these levels so effects of the stress conditions can be determined.

### Example 8

### Demonstration of Expression of a Tumorigenic Phenotype in CDNR4 Cells Exposed to Cell Crowding and Nutrient Deprivation Stress for Three Weeks

In this experiment, cell crowding and nutrient deprivation (in the form of reduced feeding of the cells as necessary for optimal cell expansion) were used as representative of two different types of stress-inducing conditions. These conditions were used to test the ability of the SR assay to detect expression of the tumorigenic phenotype in cells exposed to a prolonged, high level stress challenge. CDNR4 cells were grown in their preferred growth medium (see above) until approximately 6 million cells were available. Cells were removed from the expansion flasks, counted, and re-plated at a rate of 200,000 cells per 100 mm tissue culture dishes in growth medium. 24 hours later, the cells were re-fed with fresh growth medium.

Over the next three weeks the cells were allowed to expand without removal or addition of fresh medium and without re-plating of the cells to reduce cell density. The tissue culture dishes were examined periodically. The cells expanded until they were completely confluent and tightly packed on the tissue culture plates. Evidence of floaters (i.e., cells that had become detached from the tissue culture dishes and that were suspended in the growth medium) was visible. Cells attached to the tissue culture vessels had normal appearing, compacted cell morphology and were resistant to the stress environment.

At the end of the three week period, the stress challenge was stopped. Attached, viable cells were removed from the flasks with trypsin/EDTA, counted, re-suspended in serum-free medium, and injected in the vicinity of the inguinal mammary fat pad of female nude mice at a rate of 3 to 4 million cells per injection site. In this example, the stress challenge was removed at the end of 3 weeks and the resolution phase of the assay was conducted in vivo by injecting the cells into nude mice. The injection sites were evaluated weekly for evidence of tumor growth. Tumors appeared near the injection sites at an average of 8 to 10 weeks post-injection and grew rapidly. A total of 12 injection sites were tested; by ten weeks post-injection all 12 sites had visible tumors. In contrast, parental cells that were not exposed to the above prolonged stress challenges and that were injected at the same rate in female nude mice had a spontaneous tumorigenic rate of less than 10% and a latency of 6 to 12 months. These findings demonstrate that the prolonged stress challenge consisting of cell crowding and nutrient deprivation was resolved by survival of cells/cell clones that expressed a tumorigenic phenotype. This example demonstrates that the SR assay can be used to identify stress environments and/or agents or conditions that induce expression of a tumorigenic phenotype in cells of interest.

### Example 9

### Demonstration of Low-Dose X-Ray-Mediated Prevention and/or Protection Against Expression of Error-Prone Stress Response Resolution Pathways in Immortalized Murine Lung Epithelial Cells by 6-TG

T273 cells, a subclone of C10 cells (moderately tumorigenic murine alveolar type II cells) were grown in their preferred medium until approximately 6 million cells were available. Cells were removed from the expansion flasks, counted, and replated at a rate of 500,000 cells per 75 mm flask in growth medium, 24 hours later, the cells were re-fed with fresh growth medium. Flasks were placed in a table-top x-ray machine and exposed for 5 minutes without (control cells) or with (treated cells) x-ray radiation. The estimated dose of radiation was 0 and 10 rads for control and treated cells, respectively. All exposures were performed in duplicate. Following the exposures the cells were returned to the incubator.

Over the next two weeks the cells were evaluated repeatedly and no visual effect of the exposure upon cell morphology, cell turnover rates, or cell growth rates was observed. At the end of 2 weeks, the cells were assayed using the SR assay as described above (see example #2). Cloning efficiencies, which were obtained pre-SR induction for control and treated cells, were 42.4% and 37.4% respectively, and were not significantly different. The post-SR induction survival frequencies for control cells were as follows: total fCE=4508.9×10⁻⁶, Phen-parental CE=2488×10⁻⁶, Phne-HPRT⁻ fCE=212.5×10⁻⁶, Phen-alt-rev and Phen-alt-irr=1309.0×10⁻⁶. Another surviving clone type consisted of cells that formed foci during in vitro culture; the fCE for these clones among control cells was 499×10⁻⁶. For treated cells, the survival frequencies were as follows: total fCE=375.9×10⁻⁶, Phen-parental fCE=300×10⁻⁶, Phen-HPRT⁻-fCE=74.9×10⁻⁶ Phen-alt-rev fCEs, Phen-alt-irr fCEs and focus-forming clone fCEs=<1×10⁻⁶. Note that the limit of detection of the assay in this experiment was 1×10⁻⁶.

These results show that the use of the low-dose x-ray exposure as a preventative/protective agent prevented the expression of error-prone stress response resolution pathways involved in the formation of Phen-alt-rev, Phen-alt-irr, and focus forming clones. It also reduced the fCEs of Phen-HPRT⁻ clones to less than half that found in the control cell population. The results of these studies demonstrate that the SR assay can be used to identify agents and conditions that protect against the survival of cell clones that are representative of expression of error-prone stress response resolution pathways.

### Example 10

### Comparison of the Effects of Two Drugs Upon Error-Free Versus Error-Prone Stress Resolution Pathway Expression and Functionality

CDNR4 cells were grown and exposed to drug A or drug B as described above (see example #1 for exposure methods). After 2 weeks, the SR assay was performed and total fCEs were determined as described above; SFs for Phen-parental, Phen-alt-rev, and place-alt-irr clones for drug A were 12780×10⁻⁶, 181×10⁶, and 134×10⁻⁶, respectively. Comparable values for drug B were 10007×10⁻⁶, 204×10⁻⁶, and 254×10⁻⁶, respectively. Cloning efficiencies for cells exposed to both drugs were not significantly different. These comparisons show that drug A had more desirable effects upon stress resolution pathway expression and function than drug B, with higher total survival, higher error-free pathway expression, and lower expression of error-prone stress resolution pathways. This example demonstrates the use of the SR assay for evaluating and comparing the effects upon stress resolution pathways of drugs developed for use in humans and animals.

### BRIEF DESCRIPTION OF THE TABLES AND FIGURES

Table 1: Stress response induction levels, expected stress response resolution pathway/outcomes, and some hypothesized epigenetic alterations that are proposed to correspond to the level of stress resolution response expressed by the cell population, or induced using the calibrated stress conditions.

Table 2: Cellular processes that lead to or contribute to damage avoidance stress response resolution and/or damage resolution.

Table 3: Major DNA repair pathways and their damage avoidance versus damage tolerance sub-pathways, and hypothesized mechanisms or factors that affect cell choice between arms and/or functionality of each arm.

Table 4: Types of stress and/or stress inducing agents or conditions that can be evaluated or used in the SR assay

Table 5: Criteria currently used to determine the gene expression characteristics that affect cell morphology for a cell population and/or to classify SR assay surviving clones as Phen-parental or Phen-alt. Results expected for Phen-parental cell clones are described in parentheses.

Table 6: Testing used to sub-classify SR assay surviving clones, and expected results associated with each subgroup.

Table 7: Major phases and steps in the SR assay, with the goals and endpoints evaluated in each phase.

Table 8: Systems, processes and/or pathways involved in or hypothesized to be involved in epigenetic cell memory.

Table 9: Methods for determination of epigenetic modifications/alterations in SR assay cell clones.

Table 10: Pathways, factors, elements with potential role(s) in damage avoidance pathways (not all pathways work in all cells; there appears to be cell type-specific functionality of pathways).

TABLE 1
Stress response induction levels, expected stress response resolution pathways and/or outcomes,
and hypothesized accompanying epigenetic alterations. Note that there is overlap in the pathways
that are expressed at each level, thus, the categories refer to the dominant pathway expressed.

| | | | | High and/or |
|---|---|---|---|---|
| | Very low and/or | Low and/or acute | | prolonged and/or |
| | acute level SR | level SR | Moderate level | chronic level SR |
| | induction | induction | SR induction | induction^{a} |
| Response type | “No change | Damage | Damage tol-rev | Damage tol-irr paths |
| | response” | avoidance paths | paths | |
| Known and/or | (1) Baseline gene | (1) Up-regulation | (1) Increased | 1) Down-regulation |
| hypothesized | expression levels | of DNA repair | expression of TLS | of DNA repair |
| gene expression | are sufficient to | (2) Up-regulation | pathways and | (2) Down-regulation |
| changes | resolve damage | of apoptosis | elements; TLS | of apoptotic |
| | (2) No significant | (3) Metabolic | constrained to | pathways |
| | change in gene | changes | work on induced | (3) TLS paths |
| | expression is | (4) Production of | lesions; | expressed without |
| | observed | protective | (2) Other damage | constraints |
| | | substances | tolerance | (2) Other damage |
| | | (5) Others | resolution paths | tolerance paths |
| | | | expressed? | expression |
| | | | (3) Mutator | increased |
| | | | phenotype? | (3) Some damage |
| | | | | avoidance paths |
| | | | | expression |
| | | | | decreased; |
| | | | | (4) apoptosis |
| | | | | decreased |
| | | | | (5) DNA |
| | | | | proofreading |
| | | | | decreased |
| | | | | (6) Mutator |
| | | | | phenotype |
| | | | | (7) Others? |
| Hypothesized | (1) None or | (1) Unknown, | (1)Chromatin | (1) Permanent |
| chromatin | (2) rapid, transient | Transient | memory marks, | changes in |
| changes | changes difficult to | (2) Chromatin | gene bookmarking | epigenetic-based |
| | detect | memory marks/gene | changes, | cell memory. |
| | | bookmarking | (2) TLS pathways | (2) Genomic |
| | | changes possible | expressed; | instability expressed |
| | | 3) TLS pathway(s) | constrained, | (3) TLS expressed; |
| | | not expressed or | probably act upon | acts without |
| | | expressed at very | induced lesions | constraint across the |
| | | low levels. | only. | genome. |

^{a}this pathway may consist of two major subpathways, one of which is associated with expression of a tumorigenic phenotype, and one of which is not.

TABLE 2
Cell processes/pathways composing and/or contributing to damage
avoidance stress response and/or damage resolution; additional
processes/pathways are expected to be described in the future

| | |
|---|---|
| Cell environment sensing/ | Chromatin: maintenance and |
| signaling systems | remodeling complexes |
| Cell-cell communication | Cell metabolism systems |
| DNA proofreading | Cell transport systems |
| Cell cycle checkpoints | Plasma membrane dynamics |
| DNA repair (2-3 or more | Protective substance production |
| operational levels) | |
| DNA repair elements (protective | Other SRs (ex. Mitochondrial SR) |
| functions) | |
| Apoptosis (2 kinds: intra- and | Immune surveillance |
| intercellular (Bauer) | |

TABLE 3
Major DNA repair pathways, error-free and error-prone arms, and hypothesized mechanisms
or factors that affect cell choice between arms and/or functionality of each arm.

| DNA repair pathway | Error-free | Error-prone |
|---|---|---|
| Excision repair pathways (base | + | + |
| excision repair; nucleotide | Cell choice between error-free vs | Cell choice between error-free vs |
| excision repair) | error-prone may depend upon | error-prone may depend upon |
| | levels of functional elements | levels of functional elements |
| Mismatch repair | + | + |
| | Cell choice may depend upon the | May occur through activation of |
| | function of non-MMR elements | post-replication pathways |
| Non-homologous end-joining | + | + |
| | Fast pathway; probable factors | Slow pathway: not DNA-PKcs |
| | involved: DNA-PKcs, Ku, DNA | dependent |
| | ligase IV or XRCC4 | |
| Homologous recombination | + | + |
| repair (HR) | Scheduled HR | Unscheduled HR |
| Post-replication repair | + | + |
| (RAD6/RAD18 pathways) | Ubc13/Mms2-mediated; | Translesion synthesis pathway; |
| (influenced by PCNA post- | 2 subpaths: POL30- & RAD5- | REV3, REV7 mediated; probable |
| translational modifications) | mediated; probable role for REV1 | role for REV1 (more elements may |
| | (more elements may be involved | be involved in comparable human |
| | in comparable human pathways | pathways) |

TABLE 4
Types of stress environments and/or stress inducing agents that
can be used/evaluated in the SR assay. Note that additional
stress responses are expected to be described in the future

| | |
|---|---|
| DNA damage stress | Endoplasmic reticulum stress |
| Oxidative stress | Hypoxic stress |
| Heat shock stress | Metal stress |
| Cellular microtubule stress | Physical pressure, barometric |
| | pressure or shear force stress |
| Inflammatory stress | Osmotic stress |
| Xenobiotic metabolism stress | Endogenous hormone response |
| | stress |
| Cytotoxic stress | Nutrient-deprivation or hyper- |
| | nutrition stress |
| Nucleotide pool imbalance stress | Mitochondrial stress |
| Senescence-associated stress | Microtubule stress (plasma |
| (may be related to oxidative | membrane stress) |
| stress) | |
| Cold stress | Golgi apparatus stress |

TABLE 5
Criteria used currently to determine gene expression characteristics of a cell
population and/or to classify SR assay surviving clones as Phen parental. Results
expected for Phen-parental cell clones are described in parentheses.

| Criterion | Characteristics |
|---|---|
| Cell morphology | Size, shape, retractility, cobblestone pattern, vacuolization (Phen- |
| | parental clones should resemble parental cells in all of these |
| | characteristics) |
| Cell growth | Rate, viability, monolayer growth, contact inhibition, formation of |
| | cobblestone-like monolayer for epithelium (Phen-parental clones |
| | should resemble parental cells in all of these characteristics) |
| Cell-cell communication and/or | Tight junction formation, intercellular bridge formation (Phen-parental |
| attachments | clones should resemble parental cells in all of these characteristics) |
| Metabolic changes | Medium color changes; growth in presence of toxins (raw purines, |
| | pyrimidines) (Phen-parental clones should resemble parental cells in all |
| | of these characteristics) |
| Nutrient requirements | Cells should NOT grow better/faster on medium containing raw purines |
| | than cells being expanded in growth medium alone. (Clones that grow |
| | better in the presence of raw purines are presumptive Phen-alt clones.) |
| Cell cycle checkpoint response | Cells should express a cell cycle checkpoint response; the response |
| expression | should be retained and expressed under the same conditions that |
| | induce this response in parental cells |
| Apoptosis or cell death after | Cells should undergo cell death or apoptosis. (Phen-parental cells |
| repeated SR induction | should retain and express this response under the same conditions |
| | that induce this response in parental cells.) |
| Drug sensitivity/resistance | To 6-TG, aminopterin, others; (Phen-parental cell clones show the |
| | same drug sensitivity as that of parental cells.) |
| Mutations | HPRT, PIGA, others. (Phen-parental cell clones do not have |
| | detectable mutations in these genes and/or their mutation frequencies |
| | at these loci do not exceed those of parental cells.) |

TABLE 6
Testing used to sub-classify SR assay surviving clones,
and expected results associated with each subgroup.

| | One or more | | | | Cell cycle arrest |
|---|---|---|---|---|---|
| | changes in | Growth without | Growth in guanosine | | in SR induction |
| | characteristics | further stress | or folic acid | Growth in | agent/ |
| | listed in Table 6 | induction (no test) | supplemented medium | HAT-medium | selection agent |
| Phen- | + | + | + | + | + |
| parental | | | | | |
| HPRT⁻⁻* | −−/+ | −−/+ | + | −− (+/−−) | −− |
| Phen-alt- | −− | + | + | + | −− |
| rev and | | | | | |
| Phen-alt- | | | | | |
| irr | | | | | |

*HPRT⁻⁻ cells have difficulty surviving for reasons given above, and thus, may be seen to have poor viability under all conditions except growth in guanosine supplemented medium.

TABLE 7
Major phases of the SR assay showing goals of each
phase and the endpoints evaluated in each phase.

| Assay | | | | |
|---|---|---|---|---|
| phases | Assay Phase Goals | Determinations/actions taken; assessments | | |
| Phase 1: | Pre-calibrated stress | | | |
| | conditions exppsure | | | |
| Step 1 | | Evaluate cells to be used in | Optimize growth conditions of | |
| | | the assay. | cells to be used | |
| Step 2 | May be performed as | Determine baseline DNA | Baseline | Pro-survival |
| | part of pre-SR | damage levels (if desired) | epigenetic | pathway |
| | induction | | alterations (if | expression/ |
| | testing/evaluation of | | desired) | function: CEs; |
| | cells of interest | | | SRID-x % |
| | | | | determinations |
| Step 3 | | Expose to a test agent or condition (initial exposure) (if planned as | | |
| | | part of the experimental design) | | |
| Step 4 | Midway assessments | Post-initial exposure DNA | Post-initial | SR pro-survival |
| | (performed with or | damage levels | exposure | pathway |
| | without an initial | | epigenetic | expression/ |
| | exposure step) | | damage | function: mCEs; |
| | Assessments at right | | | SRID-x % |
| | are recommended | | | determinations |
| Step 5 | | Aminopterin testing | | |
| Phase 2: | Calibrated stress | | | |
| | conditions exposure; | | | |
| | SR resolution | | | |
| Step 1 | | Exposure to calibrated stress conditions using SRID-x % level of | | |
| | | agent or condition chosen for this step | | |
| Step 2 | | Cessation of calibrated stress conditions exposure; initiation of | | |
| | | resolution | | |
| Step 3 | | Score raw data; identify viable cell clones | | |
| Phase 3: | Post-calibrated stress | | | |
| | conditions exposure; | | | |
| | clone testing for | | | |
| | phenotyping; final | | | |
| | calculations | | | |

| | |
|---|---|
| Step 1 | Completion of raw data collection; recovery of clones; clone |
| | classification |

| | | |
|---|---|---|
| DNA damage levels: HPRT- | Epigenetic | Pro-survival |
| clone fCEs (recommended) | damage: | pathway function: |
| | Phen-alt fCEs | total fCEs; fCEs |
| | | for each type of |
| | | clone as |
| | | representative of |
| | | each pathway. |

| | |
|---|---|
| Step 2 | Testing of Phen-alt clones for reversibility/irreversibility |
| Step 3 | Calculation of fCEs for each phenotypically distinct surviving clone |
| | subgroup |
| Step 4 | Stress response resolution pathway evaluations |
| Step 5 | Final surviving cell population composition and damage burden |
| | assessments |
| Step 6 and | Additional assessments: described in the text |
| following | |

TABLE 8
Systems/processes known or hypothesized to be involved in ECM

| | |
|---|---|
| RNA directed DNA methylation | Specific binding sites on genes or introns |
| Expression of DNA demethylases | Histone H3K9 methylation |
| Polycomb group proteins | Systems that maintain bivalent histone |
| | modifications |
| Transcriptional state modifying agent and/or | RNA and DNA editing via intracellular transport |
| events such as internal and external cues that | and modulation of RNAs and RNA regulatory |
| affect gene expression and post-translational | complexes and trafficking of functional RNA |
| processing; | subclasses. |
| Retention of chromatin bookmarks during mitosis | TBP transcription factor and chromatin |
| and associated systems. | bookmarking functions |
| Propagation of specific chromatin | Chromatin remodeling and maintenance systems |
| structures/configurations | |
| Systems and processes involved in the formation | RNA directed epigenetic processes - DNA |
| and persistence of small noncoding nucleic acids | methylation, nucleosome repositioning, higher |
| (ncNAs; <200 nucleotides) and similar complexes | order chromatin remodeling |
| with nucleotides | |

TABLE 9
Methods for determination of epigenetic modifications/alterations in SR assay cell clones.

| | |
|---|---|
| Chip-on-chip | Immunofluorescent labeling |
| Cleavage and/or digestion of chromatin | Immunofluorescent microscopy |
| Chromatin immunoprecipitation | In situ DNAse I sensitivity assay |
| Chromatin immunoprecipitation-on-chip | Methylated-CpG island recovery assay |
| | (MIRA) |
| Quantitative chromatin immunoprecipitation | Methylation-specific polymerase chain |
| | reaction |
| Chromatin immunoprecipitation with whole-genome | Microarray chips for methylation changes |
| scanning approaches | |
| Chromatin immunoprecipitation with ultra high- | Microarray technologies |
| throughput sequencing | |
| Chromatin immunoprecipitation and DNA microarrays | Microscopy approach for DDR-associated |
| | changes |
| Detection method for nucleosomes released from dying cells | Microscopy with antibody staining |
| Electron paramagnetic resonance with protein-specific | Nuclear texture analysis |
| spin label | |
| Epigenetic code mapping | Nucleosome mapping technique |
| Fluorescent-tagged proteins or histones | Radiolabeling |
| Fluorescent microscopy | Restriction endonucleases to define |
| | nucleosome organization |
| Flow- and laser-scanning cytometry | Restriction enzyme accessibility assay |
| Footprinting and chromatin analysis | Transcription assay with 5′fluorouridine |
| | incorporation |
| Genomic footprinting of permeabilized cells | Transcriptional profiling techniques |
| Genome-wide location analysis | Restriction endonucleases to define |
| | nucleosome organization |
| Genome-wide histone modification profiles | |
| Genome wide DNA methylation analysis | |

TABLE 10
Pathways, factors, elements with potential role in damage avoidance paths: (not all pathways
work in all cells; there appears to be cell type specific functionality of pathways.)

| Cell type identified with | |
|---|---|
| pathway | Pathway mediators, factors, &/or elements |
| Peripheral blood lymphocytes | Raf/MEK/ERK kinase cascade and PI3K/Akt/p70 ribosomal S6 kinase |
| | pathway |
| Peripheral blood lymphocytes | MAPKs (mitogen-activated protein kinases), PKA (protein kinase A) |
| | and mTOR pathways |
| Peripheral blood cells | Akt/GSK3 kinase pathway |
| Hematopoietic cells | PI-3K/AW and Raf/MEK/ERK pathways |
| Eye lens epithelial cell survival | PI-3K (phosphatidylinositol 3-kinase)/Akt alone (in eye lens epithelial |
| | cell survival) |
| Hepatocytes | PI-3K (phosphoinositide 3-kinase) and MAP (mitogen-activated |
| | protein) kinase pathways |
| Prostate cells | JNK (c-JUN NH2-terminal kinase), PKB (protein kinase B), and |
| | extracellular signal-regulated kinase pathways |
| Human ovarian cancer cells | PI-3K/AKT/mTOR (mammalian target of rapamycin)/p70S6K1 |

Other pathways that are functional in a variety of cell types:

| | |
|---|---|
| MAPKs (involved in pro- | AP-1 (activator protein-1), JNK, p38 MAPK, p44/p42 MAPK, and Akt |
| survival and anti-survival | |
| paths) | |
| | ERK, Akt, lack of activation of JNK and MAPK, and variable activation |
| | of PLCgamma2 (phospholipase Cgamma2) and NF-kappaB, and Mcl-1 |
| | (myeloid cell leukemia-1 protein) |
| | EGFR (epidermal growth factor receptor) and its targets |
| | CREB (CRE-binding protein) and c-IAP2 (cellular inhibitor of apoptosis |
| | protein 2), along with ERK1/2 (extracellular signal-related kinase1/2) |
| | and p38 MAPK |
| | IkappaBalpha, JNK, Akt, and p44/p42 MAPK |

AKT: protein kinase B

### REFERENCES CITED

Adamson et al., 1993, Biochem Pharmacol. 46:1627-36
Aggarwal et al., 2009, J. Cell Biochem. 106:758-63
Alpi et al., 2009, DNA Repair (Arnst) 8:430-5
Ayoub et al., 2009, Cell Cycle 8:1494-500
Ayoub et al., 2009, Cell Cycle 8:2945-50
Babar et al., 2008, Future Oncol, 4:289-98
Bachman et al., 2006, Toxicol. Sci. 91:393-405
Baker et al., 2008, Mutat. Res. 647:3-12
Ball et al., 2009, J. Cell Biol. 185:573-5
Barcellos-Hoff et al., 2009, Breast Cancer Res. 11:202
Burma et al., 2008, Cell Stem Cell. 2:108.40
Bartek et al., 2007, Oncogene 26:7773-9
Bartek et al., 1997, Exp. Cell Res. 237:1-6
Bartek et al. 2007, Curr. Opin. Cell Biol. 19:238-45
Bartek et al., 2007, Cell Cycle 6:2344-7
Barzilai et al., 2008, DNA Repair (Arnst) 7:1010-27
Belyakov et al., 2002, Radiat. Prot. Dosimetry 99:249-51.
Bennett et al., 2009, Methods 48:63-71
Bitomsky et al., 2009, FEBS Lett. 276:6074-83
Bolderson et al., 2009, Clin. Cancer Res. 15:6314-20
Bonatto et al., 2007, Free Radic. Biol. Med. 43:557-67
Bourdeau et al., 2009, Front. Biosci. 14:475-85
Bronner et al., 2007, Curr. Med. Chem. 14:2629-41
Burhans et al., 2009, Free Radic. Biol. Med. 47:1282-93
Casorelli et al., 2008, Anticancer Agents Med. Chem. 8:368-80
Cejka et al., 2008, Genetics 179:1835-44
Cohn et al., 2008, Mol. Cell, 32:306-12
d'Adda di Fagagna, 2008, Nat. Rev. Cancer 8:512-22
Dantzer et al., 2006, Methods Enzymol. 409:493-510
Das et al., 2008, Cell Cycle 7:154-7
Daugaard et al., 2007, FEBS Lett, 581:3702-10
Davidson, 2009, DNA Repair (Arnst) 8:1110-7
De Boeck et al., 2009, J. Pathol. 217:327-44
Dehennaut et al., 2009, Bull. Cancer 96:E66-72
Delcuve et al., 2008, J. Cell. Biochem. 105:1-8
Delmas et al., 2006, Proc. Natl. Acad. Sci. USA 103:4564-9
Denchi, 2009, DNA Repair (Arnst) 8:1118-26
Diaz et al., 2003, Mol. Cancer Res. 1:836-47
Dinant et al., 2009 Mol. Cell. Biol. 29:6335-40
Dobrovolsky et al., 1999, Mutat. Res. 423:125-36
Eilertsen et al., 2007, Anim. Reprod. Sci. 98:129-46
Eller et al., 1997, Proc. Natl. Acad. Sci. USA. 94:12627-32
Englander et al., 2008, Mech. Ageing Dev. 129:475
Evan et al., 2009, Curr. Opin. Genet. Dev. 19:25-31
Felberbaum et al., 2007, Cell Cycle 7:52-6
Fink et al., 1995, Clin. Cancer Res. 4:1-6
FitzGerald et al., 2009, Biochem. Soc. Trans. 37:897-904
Fomace et al., 1992, Ann. NY Acad. Sci. 663:139-53
Galhardo et al., 2009, Genetics 182:55-68
Gari et al., 2009, Crit. Rev. Biochem. Mol. Biol. 44:292-325
Gamer et al., 2009, DNA Repair (Arnst) 8:1025-37
Gonzalez et al. 2008, PLoS Genet, 4:e1000208
Gorospe et al., 2008, Trends Cell Biol. 18:77-83
Gu et al., 2009, Cell Cycle 8:6-10
Guo et al., 2009, Methods 48:72-9
Hageman et al., 2001, Mutat. Res. 475:45-56
Halazonetis et al., 2008, Science 319:1352-5
Haley et al., 2009, Int. J. Radiat. Biol. 85:643-55
Hannah et al., 2009, DNA Repair (Arnst) 8:536-43
Hardy, 1892, J. Physiol. 13:309-19
Harper et al., 2007, Mol. Cell. 28:739-45
Heierhorst et al., 2008, Cell Cycle 7:2654-60
Hersh et al., 2004, Res. Microbiol. 155:352-9
Hofmann et al., 2009, DNA Repair (Arnst) 8:544-56
Hoogstraten et al., 2008, J. Cell. Sci. 121:2850-9
Horejsi et al., 2009, Cell Cycle 8:1133-7
Huen et al., 2008, Cell Res. 18:8-16
Huertas et al., 2009, Epigenetics 4:31-42
Humpal et al., 2009, Biochem. Cell. Biol. 87:243-53
Hoyer-Hansen et al., 2007, Cell Death Differ. 14:1576-82
Jackson et al., 2009, Nature 461:1071-8
Janetka et al., 2009, Expert Opin. Ther. Pat. 19:165-97
Jarvis et al., 1998, Curr. Opin. Oncol. 10:552-9
Jha et al., 2009, Mol. Cell. 34:521-33
Jin et al., 2008, Autophagy 4:563-6
Karagiannis et al., 2004, Cell. Mol. Life. Sci. 61:2137-47
Keefe et al., 2009, Reprod. Fertil. Dev. 21:10
Kehe et al., 2009, Toxicology 263:12
Kerzendorfer et al., 2009, DNA Repair (Arnst) 8:1139-52
Kharbanda et al., 1998, Oncogene 17:3309-18
Kinner et al., 2008, Nucl. Acids Res. 36:5678-94
Kloc, 2008, Int. J. Dev. Biol. 52:115-9
Kondratov et al., 2007, Trends Cell Biol. 17:311-7
Kondratov, 2007, Ageing Res. Rev. 6:12-27
Kruman et al., 2008, Front. Biosci. 13:2504-15
Kumar, 2009, Nat. Rev. Cancer 9:897-903
Langie et al., 2007, DNA Repair (Arnst) 6:852-62
Lavin, 2008, Nat. Rev. Mol. Cell. Biol. 9:759-69
Li et al., 2005, J. Biol. Chem. 280:37948-56
Li et al., 2002, Mutat. Res. 510:71-80
Liang et al., 2009. World J. Surg. 33:661-6
Liew et al., 2009, Arch. Toxicol. 83:407-16
Lisby et al., 2009, DNA Repair (Arnst) 8:1068-76
Liu et al., 2009, Curr. Opin. Immunol. 21:431-9
Loeb et al., 2001, Cancer Res. 61:3230-9
Loeb et al., 2003. Proc. Natl. Acad. Sci. USA, 100:776-81
Longhese, 2008, Genes Dev. 22:125-40
Martin, 2008, J. Neuropathol. Exp. Neurol. 67:377-87
Matsuda et al., 2009, Med. Mol. Morphol. 42:138-42
Mazumder et al., 2007, Cell Cycle 6:1437-42
McKenna et al., 2009, Epigenetics and cancer without genomic instability. 8:23-6
Meek, 2009, Nat. Rev. Cancer 9:714-23
Mehler, 2008, Prog. Neurobiol. 86:305-341
Messick et al., 2009, J. Cell Biol. 187:319-26
Miura et al., 2008, Environ. Mol. Mutagen. 49:614-21
Miura et al., 2008, Environ. Mol. Mutagen. 49:622-30
Mimitou et al., 2009, DNA Repair (Arrest) 8:983-95
Mimitou et al., 2009, Trends Biochem. Sci. 34:264-72
Misri et al., 2008, Cytogenet. Genome Res. 122:297-307
Mohammad et al., 2009, DNA Repair (Arnst) 8:1009-17
Morel et al., 2009, J. Cell. Mol. Med. 13:1476-88
Morio et al. 2008, Int. J. Biochem. Cell Biol. 40:598-603
Murphy et al., 2008. Exp. Cell Res. 314:2224-30
Murray et al., 2004, Mol. Biol. Cell. 15:2361-74
Nakajima et al., 2006, J. Biol. Chem. 281:34687-95
National Research Council (NRC). 2007, Toxicity Testing in the 21st Century: A Vision and Strategy. Committee on Toxicity Testing and Assessment of Environmental Agents. The National Academies Press, Washington, D.C.
Driscoll, 2009, DNA Repair (Arnst) 8:1333-7
Okumura et al., 2009, Cancer Sci. 100:1145-50
Orcesi et al., 2009, Br. Med. Bull. 89:183-201
Pandita et al. 2009, Crit. Rev. Eukaryot. Gene Expr. 19:235-51
Panier et al., 2009, DNA Repair (Arnst) 8:436-43
Peng et al., 2007, Mol. Cell. Biol. 27:6852-62
Polo et al., 2007, Cell Cycle 6 (19)
Ponder et al., 2005, Mol. Cell. 19:791-804
Powell et al., 2009, DNA Repair (Arnst) 8:1153-65
Prives et al., 2008, Cell Cycle 7:3840-6
Reed et al., 2009, Future Oncol. 5:713-26
Rego et al., 2009, Mutat. Res. 668:27-41
Reineke et al., 2009, Int. J, Biol. Sci, 5:366-76
Reinhardt et al., 2009, Curr. Opin. Cell Biol. 21:245-55
Rog et al., 2008, Curr. Opin. Genet. Dev. 18:212-20
Sarge et al., 2009, Cell Cycle 8:818-23
Sawan et al., 2008, Mutat. Res. 642:1-13
Schreiber et al., 2002, Cell 111:771-8
Shore et al., 2009, EMBO J. 28:2309-22
Simon et al., 2008, Proc. Natl. Acad. Sci. USA, 105:1152-7
Slijepcevic, 2008, Mech. Ageing Dev. 129:11-6
Soutoglou et al., 2008, Science 320:1507-10
Srivastava et al., 2009, Mutat. Res. 681:180-8
Stehmeier et al., 2009, DNA Repair (Arnst) 8:491-8
Stracker et al., 2009, DNA Repair (Arnst) 8:1047-54
Strauss, 1998, Semin. Cancer Biol. 8:431-8
Tang et al., 1998, Proc. Natl. Acad. Sci. USA 95:9755-60
Tesniere et al., 2008, Cell Death Differ. 15:3-12
Thierbach et al., 2009, Anal. Bioch. 387:318-320
Ting et al., 2006, Genes Dev. 20:3215-31
Toyoshima, 2009, J. Radiat. Res. (Tokyo). 5011-7
van Attikum et al., 2009, Trends Cell Bil. 19:207-17
Weinstein et al., 2004, Stroke 35:2666-70
Yang et al., 2003, Carcinogenesis 24:1571-80
Yang et al., 2004, World J. Gastroenterol. 10:155-60
Yata et al., 2009, DNA Repair (Arnst) 8:6-18
Yeung et al., 2008, Genome Biol. 9:227
Yin et al., 2008, Immunol Res. 42:29-40
Wilkerson et al., 2007, Cell Stress Chaperones 12:283-90
Zhu et al., 2003, World J. Gastroenterol. 9:888-93

The disclosure of every patent, patent application, and publication cited herein is hereby incorporated herein by reference in its entirety.

While this subject matter has been disclosed with reference to specific embodiments, it is apparent that other embodiments and variations can be devised by others skilled in the art without departing from the true spirit and scope of the subject matter described herein. The appended claims include all such embodiments and equivalent variations.

What is claimed is:

1. A method of assessing the potential of an external stimulus to modulate a stress response resolution pathway (SRRP) in a metazoan cell of a selected type, the method comprising:
A) subjecting a metazoan cell of the selected type to a first incident of the stimulus; thereafter
B) subjecting the cell to a calibrated stress condition (CSC) wherein the CSC is selected such that a substantial fraction of control cells of the same type that have not been subjected to any substantial stress exhibit a reproducible response to the CSC; and thereafter
C) assessing the difference between
i) the response to the CSC of the cell subjected to the stimulus and
ii) the response to the CSC of the control cells,
whereby a difference between i) and ii) indicates that the stimulus modulates stress response in cells of the selected type.

2. The method of claim 1,
wherein the SRRP is selected from the group consisting of pro-survival, damage avoidance (PSDA) SRRPs; pro-survival damage tolerance (PSDT) SRRPs; and pro-death SRRPs; and
wherein the difference between
i) the response to the CSC of the cell subjected to the stimulus is a characteristic of the selected SRRP in the cell and
ii) the response to the CSC of the control cells is the same characteristic of the selected SRRP in the control cells,

3. The method of claim 2, wherein the characteristic is selected from the group consisting of
characteristics of mitochondrial alterations;
characteristics of DNA mutation processes;
characteristics of epigenome alterations;
characteristics of RNA-directed DNA methylation processes;
characteristics of DNA demethylation processes;
characteristics of Histone H3K9 methylation processes;
characteristics of RNA-directed histone modification processes;
characteristics of RNA-directed nucleosome repositioning processes;
characteristics of RNA-directed chromatin remodeling processes;
characteristics of transcriptional state modification processes;
characteristics of RNA editing processes modulated by intracellular transport and modulation of RNA-containing components;
characteristics of DNA editing processes modulated by intracellular transport and modulation of RNA-containing components;
characteristics of processes for propagation of chromatin structure;
characteristics of processes for propagation of chromatin configurations;
characteristics of processes for maintenance of bivalent histone modifications;
characteristics of chromatin bookmarking processes;
characteristics of immune system antigenic memory processes;
characteristics of processes for expression of polycomb group proteins;
characteristics of processes for binding of polycomb group proteins with nucleic acids;
characteristics of processes for micronRNA-mediated gene expression;
characteristics of processes for expression of small non-coding nucleic acids; and
characteristics of processes for expression of ribonucleoproteins.

4. The method of claim 2, wherein the SRRP is a pro-death SRRP and the characteristic is selected from the group consisting of cell survival and cell death.

5. The method of claim 1, further comprising assessing a direct effect of the stimulus on the cell after subjecting it to the stimulus and prior to subjecting it to the CSC.

6. The method of claim 1, wherein a plurality of cells of the same type are subjected to the stimulus prior to subjecting the plurality of cells to the CSC.

7. The method of claim 6, wherein substantially all of the cells of the plurality are present in the form of single cells.

8. The method of claim 6, further comprising isolating from the plurality cells that exhibit a difference between i) and ii) after the plurality is subjected to the CSC.

9. The method of claim 1, wherein the cell and the control cells are human cells.

10. The method of claim 1, wherein the response of control cells to the CSC is substantially the same among all control cells.

11. The method of claim 1, wherein a characteristic is assessed collectively for groups of control cells and the response of the groups to the CSC is that about half of the groups exhibit one binomial state of the characteristic and the remainder of the groups exhibit the other binomial state of the characteristic.

12. The method of claim 1, wherein a characteristic is assessed collectively for groups of control cells and the response of the groups to the CSC is that at least about a third of the groups exhibit one binomial state of a characteristic and the remainder of the groups exhibit the other binomial state of the characteristic.

13. The method of claim 1, wherein the response of control cells to the CSC is that at least about a tenth of the control cells exhibit one binomial state of a characteristic and the remainder of the control cells exhibit the other binomial state of the characteristic.

14. The method of claim 1, wherein the external stimulus is contact with a composition.

15. The method of claim 14, wherein the composition comprises a pharmaceutically active agent.

16. The method of claim 1, wherein the stimulus is exposure to an agent suspected of having a deleterious effect on health of the metazoan.

17. The method of claim 1, wherein the stimulus is exposure to an agent suspected of having a beneficial effect on health of the metazoan.

18. The method of claim 1, wherein the cell is of a type selected from the group consisting of glial cells, sensory neurons, motor neurons, interneurons, cardiomyocytes, dendritic cells, fibroblasts, fibrocytes, multipotent cells, endothelial cells of a single organ, endothelial cells of a single tissue, and epithelial cells.

19. The method of claim 1, wherein the cell is of a type selected from the group consisting of primary rat lung epithelial cells, immortalized murine mammary epithelial cells, immortalized murine alveolar type II lung cells, and immortalized human bronchial epithelial cells.

20. A method of assessing the potential of an external stimulus to modulate cellular stress responses in a metazoan cell of a selected type, the method comprising:
1) subjecting a population of metazoan cells of the selected type to a first incidence of the stimulus,
2) thereafter subjecting the population to a calibrated stress condition CSC), wherein the CSC is selected such that a substantial fraction of cells of the selected type that have not been subjected to the stimulus exhibit a reproducible response to the CSC, and
3) thereafter assessing the responses of cells in the population,
whereby a difference between the response of the cells of the population and the reproducible response is an indication that the stimulus modulates cellular stress responses in metazoan cells of the selected type.
